(19) United States
(12) Patent Application Publication
(10) Pub. No.: US 2022/0347278 A1
(43) Pub. Date: Nov. 3, 2022

(54) BLOOD-BASED BIOMARKERS AND USE THEREOF FOR TREATING CANCER

(71) Applicants: The Regents of the University of California, Oakland, CA (US); Dana-Farber Cancer Institute, Inc., Boston, MA (US)

(72) Inventors: Adil Daud, San Francisco, CA (US); Michael Rosenblum, San Francisco, CA (US); Meromit Singer, San Francisco, CA (US)

(21) Appl. No.: 17/732,926

(22) Filed: Apr. 29, 2022

Related U.S. Application Data
(60) Provisional application No. 63/181,497, filed on Apr. 29, 2021.

(51) Int. Cl. A61K 39/00; C12N 5/0783

(52) U.S. Cl. CPC A61K 39/0011; C12N 5/0638; A61K 2039/5158; A61P 35/00

(57) ABSTRACT

Provided herein are, inter alia, methods of treating cancer in patients who have certain biomarkers in their blood, including administering T cells containing the blood-based biomarkers to the patients and treating and identifying cancer patients having T cells which contain the blood-based biomarkers.

### CROSS-REFERENCE TO RELATED APPLICATIONS

This application claims priority to U.S. Application No. 63/181,497 filed Apr. 29, 2021, the disclosure of which is incorporated by reference herein in its entirety.

### BACKGROUND

Cancer immunotherapy has revolutionized treatment of many solid and liquid tumors (Chen and Mellman, 2017; Ribas and Wolchok, 2018; Sharma and Allison, 2015; Sharpe and Pauken, 2017; Sun et al., 2018). The systemic immune response is critical for anti-tumor immunity following checkpoint blockade (Fransen et al., 2018; Huang et al., 2019; Huang et al., 2017; Kamphorst et al., 2017; Sharpe and Pauken, 2017; Spitzer et al., 2017; Valpione et al., 2020; Wei et al., 2019; Wu et al., 2020). Recruitment of new CD8⁺ T cells from the circulation into the tumor, termed “clonal replacement,” is associated with better responses to immunotherapy (Cloughesy et al., 2019; Valpione et al., 2020; Wu et al., 2020; Yost et al., 2019). Blood is a major site of CD8⁺ T cell trafficking between secondary lymphoid organs, primary tumors, and metastatic sites (Masopust and Schenkel, 2013), making it an ideal location to interrogate peripheral anti-tumor responses. Studies have profiled T cells in the blood of cancer patients, including during checkpoint blockade (Chalabi et al., 2020; Huang et al., 2019; Huang et al., 2017; Kamphorst et al., 2017; Twitty et al., 2020; Valpione et al., 2020; Wei et al., 2019; Wei et al., 2017; Wu et al., 2020). However, improved methods to identify T cells directed against tumors are needed to focus analyses to the minority of circulating T cells that has prognostic and functional relevance.

Tracking antigen-specific T cells in the blood is difficult because of their small number and limited reagents for tracking. Tetramers have been the gold standard for identifying antigen-specific T cells, but have limitations, including: (1) antigen must be known, (2) limited available MEW haplotypes for tetramer reagents, and (3) inefficient binding to low affinity TCRs (Jenkins et al., 2010; Martinez and Evavold, 2015). In humans, surrogate markers like Programed Death 1 (PD-1, also known as CD279) and B-and-T Lymphocyte Attenuator (BTLA) have been used to enrich the anti-tumor response in blood because of associations with exhaustion in cancer (Gros et al., 2016; Gros et al., 2014; Huang et al., 2019; Huang et al., 2017; Kamphorst et al., 2017; Twitty et al., 2020; Yan et al., 2018). However, PD-1 is not an exhaustion-specific marker. PD-1 is also at least transiently expressed on all T cells upon activation, and PD-1⁺ T cells are found in the blood of healthy people (Duraiswamy et al., 2011; Sharpe and Pauken, 2017; Wherry and Kurachi, 2015). Consequently, improving methods to allow routine, unbiased tracking of tumor-specific T cells in blood would bring substantial statistical power and biological precision to analyses of anti-tumor responses. The disclosure is directed to these, as well as other, important ends.

### BRIEF SUMMARY

The disclosure provides methods of treating cancer in a patient in need thereof by isolating tumor-matching T cells ex vivo from blood obtained from the patient, thereby producing isolated tumor-matching T cells; expanding the isolated tumor-matching T cells ex vivo, thereby producing expanded tumor-matching T cells; and administering the expanded tumor-matching T cells to the patient. In embodiments, the tumor-matching T cells have a decreased expression level of one or more of LTB, CCR7, GYPC, and FLT3LG relative to a control; an increased expression level of one or more of KLRD1, NKG2D, and KLRK1 relative to a control; and an increased expression level of one or more of CXCR3, CD39, LGAS1, and LGALS3 relative to a control.

The disclosure provides pharmaceutical compositions comprising expanded tumor-matching T cells and a pharmaceutically acceptable excipient; wherein the expanded tumor-matching T cells are made by a process comprising the steps of isolating tumor-matching T cells ex vivo from blood obtained from a patient, thereby producing isolated tumor-matching T cells; and expanding the isolated tumor-matching T cells ex vivo, thereby producing expanded tumor-matching T cells. In embodiments, the tumor-matching T cells have a decreased expression level of one or more of LTB, CCR7, GYPC, and FLT3LG relative to a control; an increased expression level of one or more of KLRD1, NKG2D, and KLRK1 relative to a control; and an increased expression level of one or more of CXCR3, CD39, LGAS1, and LGALS3 relative to a control.

The disclosure provides methods of treating cancer in a patient in need thereof by administering to the patient an effective amount of an immunotherapeutic agent; wherein the patient has tumor-matching T cells with a decreased expression level of one or more of LTB, CCR7, GYPC, and FLT3LG relative to a control and/or an increased expression level of one or more of KLRD1, NKG2D, KLRK1, CXCR3, CD39, LGAS1, and LGALS3 relative to a control.

The disclosure provides methods of treating cancer in a patient in need thereof by measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, FLT3LG, or a combination of two or more thereof; comparing the expression level of the one or more genes on the tumor-matching T cells to a control; identifying the patient as being responsive to immunotherapy when the expression level of one or more of LTB, CCR7, GYPC, and FLT3LG is decreased relative to the control and/or when the expression level of one or more of KLRD1, NKG2D, KLRK1, CXCR3, CD39, LGAS1, and LGALS3 is increased relative to the control; and administering to the patient who has been identified as being responsive to immunotherapy an effective amount of an immunotherapeutic agent.

The disclosure provides methods of identifying a cancer patient who will be responsive to treatment with immunotherapy by measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, FLT3LG, or a combination of two or more thereof; comparing the expression level of the one or more genes on the tumor-matching T cells to a control; and identifying the patient as being responsive to immunotherapy when the expression level of one or more of LTB, CCR7, GYPC, and FLT3LG is decreased relative to the control and/or when the expression level of one or more of KLRD1, NKG2D, KLRK1, CXCR3, CD39, LGAS1, and LGALS3 is increased relative to the control

These and other embodiments of the disclosure are provided in more detail herein.

### BRIEF DESCRIPTION OF THE DRAWINGS

FIGS. 1A-1I: Single-cell RNA seq of CD8⁺ T cells identifies MC38 tumor-matching clones in blood based on TCR sequence. FIG. 1A: FACS plots showing PD-1 and CD44 protein in MC38 tumors and paired blood on d21. Gated on singlets, live, CD45⁺, CD8α⁺ cells. Frequency of parent expressing PD-1 shown. Data representative of 4 independent experiments, each with n=5-9 mice. FIG. 1B: Experimental design for single cell RNA seq. FIG. 1C: Clustering and UMAP visualization of paired blood (n=10,289 cells) and MC38 tumors (n=8,450 cells) on d18⁺, integrated from 3 mice (M1-3) from 2 experiments. Colors denote transcriptional clusters, labeled with functional annotations. FIG. 1D: UMAP showing CD8⁺ T cells that have a TCR matching to CD8⁺ T cells found in tumor (TM cells), colored by each mouse. Grey indicates non-matching cells (non-TM cells). FIG. 1E: Selected signatures associated with genes up regulated in TM cells or non-TM cells in blood. Significance determined using a GSEA PreRanked analysis. Full list in Table S3. FIG. 1F: UMAP showing a CD8⁺ activation signature in blood (Top). Violin plots quantifying this enrichment (Bottom). Significance determined using a Wilcoxon rank-sum test, p=1.1×10⁻⁴¹. FIG. 1G: UMAP showing clonal expansion in the blood (Top). Box plot quantifying clonal expansion (Bottom). Boxes show the first quartile, median, and third quartile, while the whiskers cover 1.5 times the interquartile range. Significance determined using a Wilcoxon rank-sum test, p=4.6×10⁻⁷. FIG. 1H: Frequency of Pdcdt cells in the blood. FIG. 1I: ROC curve showing the sensitivity and specificity of Pdcd1, Btla, Ctla4, Havcr2, Lag3, Cd160, or Tigit to distinguish TM cells from non-TM cells. The AUCs values: Pdcd1=0.548, Btla=0.486, Ctla4=0.535, Havcr2=0.500, Lag3=0.556, Cd160=0.574, Tigit=0.603. The dashed line (on the diagonal) represents the sensitivity and specificity values of random chance.

FIGS. 2A-2I: Cell-surface marker panels can enrich tumor-matching cells from blood. FIGS. 2A-2B: Logistic regression showing classification of cells as TM or non-TM based on (FIG. 2A) all genes and (FIG. 2B) a pre-selected list enriched for surface-marker genes (Chihara et al., 2018). Shown are the first two principal component projections (left), ROC curves (middle) and the Recall-Precision plots (right) with 5-fold cross validation. FIG. 2C: Top 20 surface markers by q-value for identifying TM cells in the blood using COMET. Significance determined using an XL-minimal hypergeometric test with multiple hypothesis test corrections. Full list in Table S4. FIG. 2D: Biological functions for positive markers (q-value<=0.01) identified using COMET for TM cells. FIG. 2E: Frequency protein⁺ of CD8⁺ T cells in the blood of mice with MC38 tumors at d21 (n=9 mice) by flow cytometry. Gated on singlets, live, CD45⁺, CD8α⁺. Representative of 2-4 independent experiments depending on the marker, each with n=5-9 mice. Bars show the mean, and error bars represent SD. FIG. 2F: FACS plots showing CD39, NKG2D, and CX3CR1 (Y axis) as indicated above each plot, and CD44 (X axis) on CD8⁺ T cells in the blood of mice in (FIG. 2E). FIG. 2G: UMAP visualization of mice from the validation cohort. Far left shows cells colored by matching status (green=TM, grey=non-TM). The three UMAPs to the right show cells colored by protein (NKG2D, CD39, and CX3CR1) using CITE seq (red=positive, grey=negative). Significance for CD39: p=3.87×10⁻⁵⁴ and p=7.53×10⁻⁷¹, NKG2D: p=3.19×10⁻¹²² and p=1.93×10⁻¹⁷⁵, CX3CR1: p=9.22×10⁻¹⁷ and p=2.08×10⁻³⁰ for Mouse 4 (M4) and Mouse 5 (M5) respectively, assessed using a Wilcoxon rank-sum test. FIG. 2H: ROC curves showing the sensitivity and specificity of each protein at identifying TM cells. FIG. 2L Sensitivity and specificity for proteins in identifying TM cells as single markers or two- and three-protein combinations, colored black if they are Pareto-optimal (e.g. no other gate with strictly better sensitivity and specificity), grey if not Pareto-optimal. The “&” indicates an “and” gate, and the “|” indicates an “or” gate. Full list of values in Table S5.

FIGS. 3A-3E: Tumor-matching CD8⁺ T cells in blood are less dysfunctional than the corresponding clones in tumor. FIG. 3A: CD8⁺ T cells from the integrated MC38 tumor samples colored by matching status. Navy blue=blood-matching cells, grey=non-matching cells. FIG. 3B: The distribution of cells across the transcriptional clusters in blood (Top) and MC38 tumors (Bottom). Shown is the percentage of each cluster that is matching vs. non-matching. Shown are clusters with more than 50 cells. FIG. 3C: UMAP visualization showing clonal expansion across CD8⁺ T cells from tumor as measured by clone size (Top). Box plot quantifying clonal expansion in the tumor (Bottom). Significance determined using the Wilcoxon rank-sum test, p=4.9×10⁻²⁶. FIG. 3D: Expansion rates of clones in blood and MC38 tumor (log-scale, for M1). FIG. 3E: Enrichment scores for a “terminal exhaustion” signature (p=3.1×10⁻⁹) and an “effector-like” signature (p=1.1×10⁻⁹) in tumor and blood. Significance determined using a Wilcoxon signed-rank test. Each dot shows the average gene signature of the cells in a given clone, and lines connect the same clone between blood and tumor samples. Shown are clones detected in M1. M2 and M3 shown in FIG. 9D.

FIGS. 4A-4F: Tumor-matching CD8⁺ T cell clones can be detected in the blood of metastatic melanoma patients and show less signs of dysfunction than matching clones in tumor. FIGS. 4A4B: Clustering and UMAP visualization of paired blood (n=21,833 cells) and tumor (n=16,878 cells) samples from immunotherapy-treatment naïve patients, filtered to show CD8⁺ T cells. Data are integrated from the initial blood sample from four patients (patient clinical parameters in FIG. 10A and Table S7). Colors indicate transcriptional clusters. Functional annotations of each cluster are indicated. FIG. 4C: CD8⁺ T cells in blood colored by matching status in each patient (color=TM, grey=non-TM). FIGS. 4D-4E: Enrichment of activation (FIG. 4D) or naïve (FIG. 4E) CD8⁺ T cell signatures. Significance determined using a Wilcoxon rank-sum test. For the activation signature in (FIG. 4D), p values are K409 p=7.1×10⁻⁸, K411 p=3.3×10⁻¹⁵, K468 p=3.2×10⁻¹⁰¹, K484 p=6×10⁻¹⁵. For the naïve signature in (FIG. 4E), p values are K409 p=1.1×10⁻⁷, K411 p=8.9×10⁻¹¹, K468 p=1×10⁻⁹¹, K484 p=2.9×10⁻¹². FIG. 4F: Mean value of an “exhaustion” signature in blood and in tumor. Significance determined using a Wilcoxon signed-rank test, p values are K409 p=0.2, K411 p=4×10⁻⁵, K468 p=6.5×10⁻¹⁹, K484 p=2.1×10⁻⁷. Each dot shows a clone, and lines connect the same clone between paired blood and tumor samples. For patient samples, “tumor” in the Figure refers to both resections from the primary tumor and metastases as indicated in FIG. 10B.

FIGS. 5A-5C: Matching clones can be detected in longitudinal blood samples from melanoma patients and show increased signs of exhaustion compared to clones from pre-treatment blood. FIG. 5A: Number of clones detected and overlapping between samples in the initial blood, longitudinal blood, and tumor samples of K411 and K468. FIG. 5B: Mean value of an “exhaustion” gene signature from tumor, initial paired blood, and longitudinal blood. Each dot shows a clone, and lines connect the same clone between samples. Shown are only clones that were detectable in both blood samples and the tumor sample. Significance determined using a Wilcoxon signed-rank test. For patient K411, blood vs. longitudinal blood p=0.022; blood vs. tumor p=3.5×10⁻⁴; longitudinal blood vs tumor p=8.5×10⁻⁴. For patient K468, blood vs longitudinal blood p=1.1×10⁻¹⁴, blood vs. tumor p=5.8×10⁻¹⁵, longitudinal blood vs. tumor p=0.0017. FIG. 5C: Scatter plot showing the degree of each gene's AUC for selecting TM cells from blood. Purple is a comparison between longitudinal samples from the same patient; Green is a comparison between different patients. Points outlined in black are surface-expressed genes. Significance determined using the Spearman correlation test. For patient samples, “tumor” in the Figure refers to both resections from the primary tumor and metastases as indicated in FIG. 10B.

FIGS. 6A-6H: Identification of combinations of markers for tracking tumor-matching clones across patients. FIG. 6A: Frequency of PDCD1⁺ cells (using transcript) in the initial blood sample separated by TM and non-TM cells. FIGS. 6B-6C: ROC curves showing the sensitivity and specificity of inhibitory receptor transcripts to distinguish TM cells from non-TM cells in (FIG. 6B) the initial blood samples and (FIG. 6C) in the longitudinal blood samples. Legend shared between (FIG. 6B) and (FIG. 6C). FIG. 6D: Plot showing the significance values from the COMET analysis across blood samples. Significance determined using an XL-minimal hypergeometric test with multiple hypothesis test corrections. Circles sized by AUC for sorting TM cells from non-TM cells. The y axis corresponds to the log 2(x+1) transformation of the −log 10 of the COMET q values, capped at 10. PDCD1 and consensus markers are highlighted with color. All other surface markers are grey. FIG. 6E: Overlap between the single markers detected by COMET to distinguish TM cells from non-TM cells in the blood of mice (with MC38 tumors) and patients (with melanoma). Markers included if detected as significant (q-value<0.05) in a minimum of two samples. Significance determined using a hypergeometric test, p=5.53×10⁻⁸. Additional parameters in Table S11. FIG. 6F: ROC curves for the consensus markers identified in (FIG. 6D). FIG. 6G: The sensitivity and specificity of all possible logic gates derived from combinations of genes CCR7^{low}, LTB^{low}, GYPC^{low} and FLT3LG^{low} at their COMET-determined cutoff values. Points are shaped by the number of markers used in the logical gate, and colored black if they are Pareto-optimal (if there is no gate with strictly better sensitivity and specificity) or grey if not Pareto-optimal. A dotted line through the Pareto-optimal gates represents the ROC of this combinatorial marker collection. For (FIG. 6F) and (FIG. 6G), the dashed line (on the diagonal) represents the sensitivity and specificity values of random chance. FIG. 6H: UMAP of CD8⁺ cells integrated from all patient blood samples (including longitudinal samples). Left: true tumor-matching cells as defined by matching TCR sequence in green, non-matching in grey. Right: putative TM cells as determined by the best-performing gate, [(CCR7^{low} & FLT3LG^{low} & GYPC^{low})|LTB^{low}], are colored blue, cells not expressing the markers in this gate in grey. For this combination, Sensitivity=0.751, Specificity=0.745. The symbol “&” indicates the “and” gate, and the “|” indicates the “or” gate.

FIGS. 7A-7J show transcriptional landscape of CD8+ T cells in paired peripheral blood and MC38 tumors in mice. FIGS. 7A-7B: Tables indicating details about each mouse in the discovery cohort (M1-3), including the number of cells recovered that had gene expression (GEX) data, GEX and TCR data, number of matching cells, percentage matching cells of the total sorted population, and the frequency of Pdcd1+ TM cells in peripheral blood (FIG. 7A) and MC38 tumors (FIG. 7). The samples from M1, M2, and M3 were integrated to generate an integrated blood sample and an integrated MC38 tumor sample as a discovery cohort. These three biological replicates were generated between two independent experiments (M1, experiment 1; M2 and M3, experiment 2). FIG. 7C UMAP of the integrated blood samples (top) and MC38 tumor samples (bottom) showing the distribution of each mouse in the integrated dataset (datasets combined from M1, M2, and M3). Cells from each mouse are shown in color (M1, red; M2, green; M3, blue), and the cells from the other two mice are shown in gray for each plot. FIG. 7D: UMAP of the integrated blood samples (top) and MC38 tumor samples (bottom) showing the distribution of clones shared between tissues (TM cells in blood, and bloodmatching cells in tumor). Only TM cells (green), blood-matching cells (navy blue), and nonmatching cells (gray) from each individual mouse are shown, and the cells from the other two mice in the integrated object are excluded. FIGS. 7E-7F: UMAPs showing distribution of expression of select transcripts in the integrated blood (FIG. 7E) and MC38 tumor (FIG. 7F) samples. Genes include Pdcd1 (encoding PD-1), Havcr2 (encoding Tim-3), Sell (encoding CD62L), Tcf7 (encoding TCF-1), Mki67 (encoding Ki-67), and Gzmb (encoding granzyme B). FIG. 7G Heatmap showing the fraction of cells in the integrated MC38 tumor (top) and blood (bottom) datasets with the indicated number of TCR α and β chains detected. FIGS. 7H-7J: Top: UMAP of integrated blood samples showing expression of a cell cycle signature (Kowalczyk et al., 2015; P=0.24; H), a CD8+ naive T cell signature (Kaech et al., 2002; P=4.6×10-125; I), and a TRM signature (Beura et al., 2018; P=5.5×10-60; J). Violin plots quantifying the expression of each signature in H-J in TM compared with non-TM cells in the blood (bottom). ***, P<0.001; ns, not significant. Significance determined using Wilcoxon rank sum tests. FIGS. 7C-7J: scRNAseq integrated from three biological replicates (M1-3) between two independent experiments.

FIGS. 8A-8I. Identification and validation of markers to identify TM CD8+ T cells in blood. FIG. 8A: Top surface markers for identifying non-TM cells from TM cells in the blood based on COMET (Delaney et al., 2019) analysis. Significance determined using an XL-minimal hypergeometric test with multiple hypothesis test corrections. scRNAseq integrated from three biological replicates (M1-3) between two independent experiments. FIG. 8B: Quantification of the frequency of bulk CD8+ T cells in the peripheral blood of mice with MC38 tumors on day 21 after implantation (n=9 mice) that express the indicated proteins using FACS. Cells are gated on singlets, live/dead−, CD45+, and CD8α+ and are further gated based on CD44 expression to compare CD44low and CD44high cells. **, P<0.01; ***, P<0.001. FIG. 8C: Comparison of bulk CD8+ T cells (gated on singlets, live/dead−, CD45+, CD8α+) from the peripheral blood of mice with MC38 tumors on day 21 after implantation (n=9 mice) to naive B6 mice (n=4 mice). FIGS. 8B-8C: Data are representative of two to four independent experiments depending on the marker, with n=3-4 naive mice and n=5-9 mice with MC38 tumors (days 19-22). Bars show the mean, and error bars represent SD. Significance determined using multiple t tests using the Holm-Sidak method, with α=0.05. Each row was analyzed individually, without assuming a consistent SD. Reported are the adjusted P values considering multiple tests. Significant comparisons in B are indicated with asterisks and include PD-1, P=2.6957×10-5; Lag-3, P=0.0012; TIGIT, P=0.0012; CD39, P=3.7639×10-9; NRP1, P=5.1172×10-7; CX3CR1, P=0.0002; CCR2, P=0.0012; CCR5, P=6.4414×10-10; CXCR6, P=6.1903×10-5; CD49, P=0.0002; CD29, P=1.6745×10-9; CD11a, P=1.1636×10-7; CD18, P=1.9906×10-7; NKG2D, P=1.1863×10-7; NKG2A, P=1.8567×10-7; CD94, P=1.8567×10-7; NKG2I, P=2.6625×10-6; Slamf7, P=5.06×10-13. In B, CD160 expression between CD44high and CD44low was not significant. In C, there were no significant differences between naive B6 and B6 mice with MC38 tumors. FIG. 8D: Representative FACS contour plots showing NKG2D, CD39, and CX3CR1 expression (y axis) as indicated above each plot and CD44 (x axis) on CD8+ T cells in the MC38 tumor of mice in FIG. 2F. Representative of three independent experiments, each with n=5-9 mice. FIG. 8E: UMAPs showing distribution of expression of select transcripts in the blood of M4 (top) and M5 (bottom), two biological replicates from the validation cohort (one experiment). Genes include Pdcd1 (encoding PD-1), Klrk1 (encoding NKG2D), Entpd1 (encoding CD39), and Cx3cr1 (encoding CX3CR1). FIG. 8F: Representative FACS contour plots showing all possible pairwise combinations of NKG2D, CD39, and CX3CR1 expression (as indicated in each plot) on CD8+ T cells in the blood of mice on day 21 after implantation of MC38 tumor cells. Plots are gated on singlets, live/dead−, CD45+, CD8α+. Numbers on plots indicate the percentage of cells within each quadrant of the total parent population. FIG. 8G: Quantification of the flow cytometry plots in F showing the frequency of cells expressing one, two, or three of the indicated proteins (NKG2D, CD39, and CX3CR1) determined using Boolean gating, of the population of cells expressing at least one of the markers. Shown are the average frequencies of all possible combination gates from six mice. In G, 70.5% expressed only one of the markers but not the others, 20.1% expressed only two of the markers, and 9.4% expressed all three of the markers. Data in F and G are representative of three independent experiments with five to nine mice per experiment. FIGS. 8H-8L Quantification of the frequencies of cells expressing one, two, or three of the indicated proteins (NKG2D, CD39, and CX3CR1) of the population of cells expressing at least one of the markers in the blood of M4 and M5 using the CITE seq data (two biological replicates from the validation cohort; one experiment). The frequencies of all possible combination gates on the total population of cells from the CITE seq experiment (not subsetting based on TM status; FIG. 8H) and only the TM population (FIG. 8I). FIG. 8H: 61.9% of cells expressed only one marker, 28.7% expressed only two markers, and 9.4% expressed all three markers (values averaged between M4 and M5). FIG. 8I: 28.1% of cells expressed only one marker, 52.7% expressed only two markers, and 19.2%, expressed all three markers (values averaged between M4 and M5). The pie charts in FIGS. 8G-8H share the legend to the left of FIG. 8I.

FIGS. 9A-9D. TM CD8+ T cells in the blood show stronger enrichment for effector signatures and weaker enrichment for exhaustion signatures than the corresponding clones in the tumor. FIG. 9A: Expansion rates of clones in blood and MC38 tumor (log scale), for M4 (left) and M5 (right). Shown are two biological replicates from the validation cohort (one experiment). Data from M1 from an independent experiment are shown in FIG. 3D. FIG. 9B: Top: UMAP visualization of signatures related to CD8+ T cell transcriptional states in the mouse integrated MC38 tumor samples. From left to right are signatures of terminal exhaustion from Miller et al. (2019); TRM cells from Beura et al. (2018); cell cycle from Kowalczyk et al. (2015); naive cells from Kaech et al. (2002); and bystander cells with TCRs that are not specific to the tumor from Mognol et al. (2017). Bottom: Violin plots quantifying the expression of each signature inblood-matching compared with non-blood matching clones. Significance determined using a Wilcoxon rank sum test. Colored bars beneath the violin plots indicate whether the mean is statistically greater in blood-matching cells (terminal exhaustion, P=1×10-41; TRM P=6.9×10-13), not statistically significant (cell cycle, P=0.97), or statistically greater in non-blood-matching cells (naive, P=2.2×10-65; bystander, P=0.0016). scRNAseq integrated from three biological replicates (M1-3) between two independent experiments. FIG. 9C: Shown are average gene scores per sample for mouse blood and tumor, separated by matching status. M1-3 indicate each mouse sample number (three mice between two independent experiments). For a given signature, a gene score was calculated for each cell. Shown are naive-like (Kaech et al., 2002), cell cycle (Kowalczyk et al., 2015), and the effector-like, progenitor, and terminally exhausted signatures from Miller et al. (2019). FIG. 9D: Clone-by-clone analysis examining the mean expression of an effector-like gene signature or a terminal exhaustion gene signature from Miller et al. (2019). Each dot shows the average gene signature of the cells in a given clone, and lines connect the same clone between blood and tumor samples. Shown are clones detected in M2 (top) and M3 (bottom) from one experiment. Data from M1 from an independent experiment are shown in FIG. 3E. Significance determined using a Wilcoxon signed-rank test. For M2, P=0.0084 for the effector-like signature and P=5.3×10-4 for the terminally exhausted signature. For M3, P=0.024 for the effector-like signature, and P=1.7×10-3 for the terminally exhausted signature. *, P<0.05; **, P<0.01; ***, P<0.001; ns, not significant.

FIGS. 10A-10L. Transcriptional landscape of CD8+ T cells in paired patient peripheral blood and melanoma samples. FIG. 10A: Schematic of clinical parameters for patient samples. Patients were checkpoint-treatment naive at the time of initial paired blood/tumor sampling. Subsequent course of treatment indicated. Timing of longitudinal blood sample collection for follow-up analysis in patients K468 and K411 indicated. The longitudinal sample for K468 was taken 1 mo after the initial blood sample, and during that time the patient received anti-PD-1 and anti-CTLA-4 combination therapy. The longitudinal sample for K411 was taken ˜16 mo after the initial sample, after the patient had received anti-PD-1 as a single agent followed by combination therapy with anti-PD-1 and tavokinogene telseplasmid (TAVO; Algazi et al., 2020 for TAVO monotherapy, and clinicaltrials.gov reference NCT03132675 for combination). FIG. 10B: Table indicating details regarding each patient in the cohort, including the site of tissue resection, number of cells recovered that had gene expression (GEX) data, GEX and TCR data, number of matching cells, percentage matching cells of the total sorted population, and the frequency of PDCD1+ TM cells. Each patient and time point was processed as an independent experiment for a total of six experiments (four treatment-naive blood/tumor pairs and two longitudinal blood follow-up samples). FIGS. 10C-10F: UMAP visualization of the integrated initial paired blood samples (FIGS. 10C, 10E) and melanoma samples (FIGS. 10D, 10F) showing the distribution of each patient in the integrated object. Cells are colored by patient, and the remaining cells in the integrated object are excluded from visualization. FIGS. 10C-10D indicate all cells from a given patient; FIG. 10E-10F show matching cells colored in green (TM cells in blood) or navy blue (blood-matching cells in tumor) and nonmatching cells in gray from each patient. FIGS. 10G-10H: UMAP visualizations showing the distribution of expression of select transcripts in the integrated blood (FIG. 10G) and melanoma (FIG. 10H) samples. Genes include PDCD1 (encoding PD-1), HAVCR2 (encoding Tim-3), SELL (encoding CD62L), TCF7 (encoding TCF-1), MKI67 (encoding Ki-67), and GZMB (encoding granzyme B). The data in FIG. 10C-10H are integrated from the four initial blood/tumor samples, totaling four independent experiments. FIG. 10I-10J: Violin plots showing expression of activation (FIG. 10I) or naive (FIG. 10J) CD8+ T cell signatures in TM and non-TM cells in the longitudinal blood samples from K411 and K468. Signatures derived from Akondy et al. (2017). Significance determined used a Wilcoxon rank sum test. For the activation signature in I, P=1.2×10-76 for K411 and P<0.001 for K468. For the naive signature in J, P=6.5×10-52 for K411 and P<0.001 for K468. Each longitudinal patient sample was collected and run separately, totaling two independent experiments. ***, P<0.001. FIG. 10K: Histogram showing the distribution of AUC values averaged across the six patient samples for each of the human surface markers (Chihara et al., 2018) as positive or negative indicators of TM status. Colored lines represent the AUC for CCR7, FLT3LG, GYPC, and LTB averaged across the six patient samples as negative indicators of TM status. FIG. 10L: UMAP visualizations of the top singleton marker gates in human in CD8+ cells from all six patient blood samples integrated as described in Materials and methods. In each plot, cells are colored if they pass the particular negation gate; that is, if they are selected as TM because of their low expression of the marker (labeled markerlow). For CCR7low, sensitivity=0.827, specificity=0.619; for FLT3LGlow, sensitivity=0.780, specificity=0.447; for GYPClow, sensitivity=0.339, specificity=0.819; for LTB low, sensitivity=0.725, specificity=0.716. FIGS. 10K-10L show the data integrated for all six blood samples (four initial treatment-naive samples and two longitudinal follow-up samples), totaling six independent experiments. For patient samples, “tumor” in the figure refers to resections from the primary tumor and/or metastases as indicated in FIG. 10B.

FIGS. 11A-11F. TM CD8+ T cells identified using GLIPH2 and iSMART show similar signs of activation and sensitivity/specificity rates of markers as matching cells identified based on sequence matching. FIG. 11A: Summary metrics showing the increase in frequency of CD8+ T cells classified as TM cells in each of the four treatment-naive patient samples determined using the TCR cluster-based matching method (defined as cells identified as TM using both GLIPH2 and iSMART) compared with the exact sequence matching method. FIGS. 11B-11C: Violin plots showing enrichment of activation (FIG. 11B) or naive CD8+ T cell signatures (FIG. 11C) in TM and non-TM cells on the cells identified using the TCR cluster-based matching method. Signatures derived from Akondy et al. (2017). Significance determined used Wilcoxon rank sum test. For the activation signature in FIG. 11B, P=4.3×10-8 (1(409), P=8.5×10-15 (1(411), P=5.9×10-99 (K468), P=5.4×10-14(1(484). For the naive signature in FIG. 11C, P=7.2×10-8 (1(409), P=2.7×10-11 (1(411), P=2.5×10-89 (K468), P=9.6×10-12 (K484). ***, P<0.001. FIG. 11D: Summary metrics showing the sensitivity and specificity of the PDCD1 transcript to identify TM cells from non-TM cells in the blood using exact sequence matching compared with TCR cluster-based matching. FIGS. 11E-11F: ROC curves for TM cells classified using the TCR cluster-based matching showing the sensitivity and specificity of a collection of inhibitory receptor genes (PDCD1, BTLA, CTLA4, HAVCR2, LAG3, CD160, and TIGIT; FIG. 11E), or the consensus markers for identifying TM cells (CCR7low, LTBlow, GYPClow, or FLT3LGlow, referred to as negation markers; FIG. 11F), shown for each patient. Each treatment-naive patient sample was collected and run separately, totaling four independent experiments. Each patient is plotted individually.

Color versions of the figures are available in Pauken et al, Single-cell analyses identify circulating anti-tumor CD8 T cells and markers for their enrichment, Journal of Experimental Medicine, Vol. 218, No. 4 (Mar. 2, 2021), doi.org/10.1084/jem.20200920, the disclosure of which is incorporated by reference herein in its entirety.

### DETAILED DESCRIPTION

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

“Tumor-matching T cells” or “TM cells” refer to T cells in a subject's blood that have common T cell receptors (TCRs) or markers with tumor infiltrating lympohcytes (e.g., T cells) in the subject's tumor (“tumor T cells”). The tumor-matching T cells in the subject's blood have increased activation and tissue resident memory compared to other T cells (e.g., non-tumor-matching T cells) in the subject's blood which are enriched for quiescent markers. In embodiments, tumor-matching T cells are functional, i.e., tumor-matching T cells do not exhibit exhaustion. In embodiments, tumor-matching T cells are functional when compared to tumor T cells. In embodiments, tumor-matching T cells do not exhibit exhaustion whereas the corresponding tumor T cells exhibit at least partial exhaustion. In embodiments, tumor-matching T cells exhibit less exhaustion compared to corresponding tumor T cells. In embodiments, the tumor-matching T cells are CD8+ T cells. In embodiments, the tumor infiltrating lympohcytes are CD8+ T cells. In embodiments the common T cell receptors or markers are natural killer cell biomarkers, T cell activation markers, LTB, CCR7, GYPC, FLT3LG, or combination thereof. In embodiments, the blood is peripheral blood.

A “CD8+ T cell” or “CD8 T cell” refers to a lymphocyte that expresses the CD8 glycoprotein on its surface. Examples of CD8+ T cells include cytotoxic T cells and natural killer cells. In embodiments, a CD8+ T cell is a cytotoxic T cell. In embodiments, a CD8+ T cell is a suppressor T cell. In embodiments, a CD8+ T cell is a tumor-matching T cell.

An “isolated tumor-matching T cell” refers to a tumor-matching T cell that has been separated from one or more components of its natural environment (e.g., blood).

“Tumor infiltrating T cells” refer to T cells in a subject's cancerous tumor, also referred to as “tumor infiltrating lymphocytes” or “TIL.” In embodiments, tumor T cells have at least partial exhaustion when compared to tumor-matching T cells. In embodiments, tumor t cells have full exhaustion when compare to tumor-matching T cells.

The term “immune response” refers to a response by a cell of the immune system, e.g., T cells (CD8 T cells, CD4 T cells), B cell, antigen-presenting cell, dendritic cell, monocyte, macrophage, MKT cell, NK cells, to a stimulus. In embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

The term “exhaustion” refers to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation or cell division, entrance into the cell cycle, cytokine production, cytotoxicity, trafficking, phagocytic activity, or any combination thereof. Normal input signals can include, but are not limited to, stimulation via a receptor (e.g., CD8 T cell receptor). Exhausted immune cells (e.g., CD8 T cells) can have a reduction of at least 10%, 15%, 20%, 25%, 30%, 35%, 40%, 45%, 50%, 55%, 60%, 65%, 70%, 75%, 80%, 85%, 90%, 95%, 99% or more in cytotoxic activity, cytokine production, proliferation, trafficking, phagocytic activity, or any combination thereof, relative to a corresponding control immune cell of the same type. In embodiments, an exhausted cell is an exhausted CD8 T cell. CD8 T cells normally proliferate in response to T cell receptor and/or co-stimulatory receptor stimulation, as well as in response to cytokines such as IL-2. Thus, an exhausted CD8 T cell is one which does not proliferate and/or produce cytokines in response to normal input signals. It is well known that the exhaustion of effector functions can be delineated according to several stages, which eventually lead to terminal or full exhaustion and, ultimately, deletion. In the first stage, functional T cells enter a “partial exhaustion” phase characterized by the loss of a subset of effector functions, including loss of IL-2 production, reduced TNF-α production, and reduced capacity for proliferation and/or ex vivo lysis ability, in the second stage, partially exhausted T cells enter a “partial exhaustion” phase when both IL-2 and TNF-α production ceases following antigenic stimulation and IFN-γ production is reduced. “Full exhaustion” or “terminal exhaustion” occurs when CD8 T cells lose all effector functions, including the lack of production of IL-2, TNF-α, and IFN-γ and loss of ex vivo lytic ability and proliferative potential following antigenic stimulation, A fully exhausted CD8 T cell is one which does not proliferate, does not lyse target cells (cytotoxicity), and/or does not produce appropriate cytokines, such as IL-2, TNF-α, or IFN-γ, in response to normal input signals. Such lack of effector functions can occur when the antigen load is high and/or CD4 help is low. This hierarchical loss of function is also associated with the expression of co-inhibitor immune receptors, such as PD-1, TIM-3, LAG-3, TIM-3, LAG-3, and the like.

The term “biomarker” refers to an indicator, e.g., a predictive, prognostic, and/or a pharmacodynamic indicator, which can be detected in a sample (e.g., a blood sample, a tissue sample, e.g., a tumor tissue sample). The biomarker may serve as an indicator of a particular subtype of a disease or disorder (e.g., cancer, such as melanoma or metastatic melanoma) characterized by certain molecular, pathological, histological, and/or clinical features. In embodiments, a biomarker is a gene or a set of genes. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers. Exemplary biomarkers include LTB, CCR7, GYPC, FLT3LG, natural killer cell biomarker genes (e.g., KLRD1, NKG2D, KLRK1), and T cell activation marker genes (e.g., CXCR3, CD39, LGAS1, LGALS3).

The term “biomarker signature,” “signature,” “biomarker expression signature,” or “expression signature” are used interchangeably herein and refer to a combination of biomarkers whose expression is an indicator, e.g., predictive, prognostic, and/or pharmacodynamic (e.g., the gene signature (e.g., the combination of LTB, CCR7, GYPC, FLT3LG, KLRD1, NKG2D, KLRK1, CXCR3, CD39, LGAS1, and LGALS3). The biomarker signature may serve as an indicator of a particular subtype of a disease or disorder (e.g., cancer, e.g., melanoma or metastatic melanoma) characterized by certain molecular, pathological, histological, and/or clinical features. In some embodiments, the biomarker signature is a “gene signature.” The term “gene signature” is used interchangeably with “gene expression signature” and refers to a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to a combination of polypeptides whose expression is an indicator, e.g., predictive, prognostic, and/or pharmacodynamic.

The term “natural killer cell” refers to granular lymphocytes involved in the innate immune response. Functionally, NK cells exhibit cytolytic activity against a variety of targets via exocytosis of cytoplasmic granules containing a variety of proteins, including perforin, and granzyme proteases. Killing is triggered in a contact-dependent, non-phagocytic process which does not require prior sensitization to an antigen. Human NK cells are characterized by the presence of the cell-surface markers CD16 and CD56, and the absence of the T cell receptor (CD3).

“Natural killer cell biomarker” refers to cell surface, cytosolic or nuclear markers (biomarkers) that are chacteristically expressed on natural killer cells. Exemplary natural killer cell markers include KLRD1, NKG2D, and KLRK1.

“T cell activation marker” refers to proteins (biomarkers) that are expressed upon activation of T cells. Exemplary T cell activation markers include CXCR3, CD39, LGAS1, and LGALS3.

The term “KLRD1” or “natural killer cells antigen CD94” include any of the recombinant or naturally-occurring forms of KLRD1 or variants or homologs thereof that maintain KLRD1 protein activity (e.g., within at least 50%, 80%, 90%, 95%, 96%, 97%, 98%, 99% or 100% activity compared to KLRD1). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring KLRD1 protein. In embodiments, KLRD1 is the protein as identified by UniProtKB Reference No. Q13241, a homolog, or a functional fragment thereof.

“An increased expression of KLRD1” as referred to herein is an increased expression level of KLRD1 genes expressed by tumor-matching T cells in a subject when compared to a control. KLRD1 expression levels can be measured from tumor-matching T cells from a blood sample obtained from a subject. In embodiments, the tumor-matching T cells are CD8⁺ T cells.

The term “KLRK1” or “killer cell lectin-like receptor subfamily K member 1” include any of the recombinant or naturally-occurring forms of KLRK1 or variants or homologs thereof that maintain KLRK1 protein activity (e.g., within at least 50%, 80%, 90%, 95%, 96%, 97%, 98%, 99% or 100% activity compared to KLRK1). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring KLRK1 protein. In embodiments, KLRK1 is the protein as identified by UniProtKB Reference No. Q4FJM0, a homolog, or a functional fragment thereof.

“An increased expression of KLRK1” as referred to herein is an increased expression level of KLRK1 genes expressed by tumor-matching T cells in a subject when compared to a control. KLRK1 expression levels can be measured from tumor-matching T cells from a blood sample obtained from a subject. In embodiments, the tumor-matching T cells are CD8⁺ T cells.

The term “NKG2D” or “NKG2D membrane protein” include any of the recombinant or naturally-occurring forms of NKG2D or variants or homologs thereof that maintain NKG2D protein activity (e.g., within at least 50%, 80%, 90%, 95%, 96%, 97%, 98%, 99% or 100% activity compared to NKG2D). In embodiments, the variants or homologs have at least 90%, 95%, 96%, 97%, 98%, 99% or 100% amino acid sequence identity across the whole sequence or a portion of the sequence (e.g., a 20, 25, or 50 continuous amino acid portion) compared to a naturally occurring NKG2D protein. In embodiments, NKG2D is the protein as identified by UniProtKB Reference No. I7EPK4, a homolog, or a functional fragment thereof.

“An increased expression of NKG2D” as referred to herein is an increased expression level of NKG2D genes expressed by tumor-matching T cells in a subject when compared to a control. NKG2D expression levels can be measured from tumor-matching T cells from a blood sample obtained from a subject. In embodiments, the tumor-matching T cells are CD8⁺ T cells.

The term “CXCR3” or “C—X—C chemokine receptor type 3” include any of the recombinant or naturally-occurring forms of CXCR3 or variants or homologs thereof that maintain CXCR3 protein activity (e.g., within at least 50%, 80%, 90%, 95%, 96%, 97%, 98%, 99% or 100% activity compared to CXCR3). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring CXCR3 protein. In embodiments, CXCR3 is the protein as identified by UniProtKB Reference No. P49682, a homolog, or a functional fragment thereof.

“An increased expression of CXCR3” as referred to herein is an increased expression level of CXCR3 genes expressed by tumor-matching T cells in a subject when compared to a control. CXCR3 expression levels can be measured from tumor-matching T cells from a blood sample obtained from a subject. In embodiments, the tumor-matching T cells are CD8⁺ T cells.

The term “CD39” or “ectonucleoside triphosphate diphosphohydrolase-1” or “cluster of differentiation 39” include any of the recombinant or naturally-occurring forms of CD39 or variants or homologs thereof that maintain CD39 protein activity (e.g., within at least 50%, 80%, 90%, 95%, 96%, 97%, 98%, 99% or 100% activity compared to CD39). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring CD39 protein. In embodiments, CD39 is the protein as identified by UniProtKB Reference No. P49682, a homolog, or a functional fragment thereof.

“An increased expression of CD39” as referred to herein is an increased expression level of CD39 genes expressed by tumor-matching T cells in a subject when compared to a control. CD39 expression levels can be measured from tumor-matching T cells from a blood sample obtained from a subject. In embodiments, the tumor-matching T cells are CD8⁺ T cells.

The term “LGAS1” or “galectin 1” or “lectin, galactoside-binding, soluble, 1” include any of the recombinant or naturally-occurring forms of LGAS1 or variants or homologs thereof that maintain LGAS1 protein activity (e.g., within at least 50%, 80%, 90%, 95%, 96%, 97%, 98%, 99% or 100% activity compared to LGAS1). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring LGAS1 protein. In embodiments, LGAS1 is the protein as identified by UniProtKB Reference No. P09382, a homolog, or a functional fragment thereof.

“An increased expression of LGAS1” as referred to herein is an increased expression level of LGAS1 genes expressed by tumor-matching T cells in a subject when compared to a control. LGAS1 expression levels can be measured from tumor-matching T cells from a blood sample obtained from a subject. In embodiments, the tumor-matching T cells are CD8⁺ T cells.

The term “LGAS3” or “galectin 3” include any of the recombinant or naturally-occurring forms of LGAS3 or variants or homologs thereof that maintain LGAS3 protein activity (e.g., within at least 50%, 80%, 90%, 95%, 96%, 97%, 98%, 99% or 100% activity compared to LGAS3). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring LGAS3 protein. In embodiments, LGAS3 is the protein as identified by UniProtKB Reference No. P17931, a homolog, or a functional fragment thereof.

“An increased expression of LGAS3” as referred to herein is an increased expression level of LGAS3 genes expressed by tumor-matching T cells in a subject when compared to a control. LGAS3 expression levels can be measured from tumor-matching T cells from a blood sample obtained from a subject. In embodiments, the tumor-matching T cells are CD8⁺ T cells.

The term “LTB” or “lymphotoxin-beta” or “LT-beta” include any of the recombinant or naturally-occurring forms of LTB or variants or homologs thereof that maintain LTB protein activity (e.g., within at least 50%, 80%, 90%, 95%, 96%, 97%, 98%, 99% or 100% activity compared to LTB). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring LTB protein. In embodiments, LTB is the protein as identified by UniProtKB Reference No. Q06643, a homolog, or a functional fragment thereof.

“A decreased expression of LTB” as referred to herein is a decreased expression level of LTB genes expressed by tumor-matching T cells in a subject when compared to a control. LTB expression levels can be measured from tumor-matching T cells from a blood sample obtained from a subject. In embodiments, the tumor-matching T cells are CD8⁺ T cells.

The term “CCR7” or “C—C chemokine receptor type 7” include any of the recombinant or naturally-occurring forms of CCR7 or variants or homologs thereof that maintain CCR7 protein activity (e.g., within at least 50%, 80%, 90%, 95%, 96%, 97%, 98%, 99% or 100% activity compared to CCR7). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring CCR7 protein. In embodiments, CCR7 is the protein as identified by UniProtKB Reference No. P32248, a homolog, or a functional fragment thereof.

“A decreased expression of CCR7” as referred to herein is a decreased expression level of CCR7 genes expressed by tumor-matching T cells in a subject when compared to a control. CCR7 expression levels can be measured from tumor-matching T cells from a blood sample obtained from a subject. In embodiments, the tumor-matching T cells are CD8⁺ T cells.

The term “GYPC” or “glycophorin-C” include any of the recombinant or naturally-occurring forms of GYPC or variants or homologs thereof that maintain GYPC protein activity (e.g., within at least 50%, 80%, 90%, 95%, 96%, 97%, 98%, 99% or 100% activity compared to GYPC). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring GYPC protein. In embodiments, GYPC is the protein as identified by UniProtKB Reference No. P04921, a homolog, or a functional fragment thereof.

“A decreased expression of GYPC” as referred to herein is a decreased expression level of GYPC genes expressed by tumor-matching T cells in a subject when compared to a control. GYPC expression levels can be measured from tumor-matching T cells from a blood sample obtained from a subject. In embodiments, the tumor-matching T cells are CD8⁺ T cells.

The term “FLT3LG” or “FMS-related tyrosine kinase 3 ligand” include any of the recombinant or naturally-occurring forms of FLT3LG or variants or homologs thereof that maintain FLT3LG protein activity (e.g., within at least 50%, 80%, 90%, 95%, 96%, 97%, 98%, 99% or 100% activity compared to FLT3LG). In embodiments, the variants or homologs have at least 90%, 95%, 96%, 97%, 98%, 99% or 100% amino acid sequence identity across the whole sequence or a portion of the sequence (e.g., a 20, 25, 50, 100, 150 or 200 continuous amino acid portion) compared to a naturally occurring FLT3LG protein. In embodiments, FLT3LG is the protein as identified by UniProtKB Reference No. P49771, a homolog, or a functional fragment thereof.

“A decreased expression of FLT3LG” as referred to herein is a decreased expression level of FLT3LG genes expressed by tumor-matching T cells in a subject when compared to a control. FLT3LG expression levels can be measured from tumor-matching T cells from a blood sample obtained from a subject. In embodiments, the tumor-matching T cells are CD8⁺ T cells.

The terms “expression level,” “amount,” or “level,” or used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a posttranslational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having cancer (e.g., melanoma) who may be likely to respond to, or benefit from, a particular therapy (e.g., immunotherapy).

“Subject responsive to immunotherapy” refers to a subject that responds to treatment when administered an immunotherapeutic agent. “Responsive” and “responds” indicate that the subject has an increase over baseline of one or more of CD8+ cell infiltration, T cell activation, interferon-gamma pathway gene expression, and T cell clone expansion; a cancerous tumor does not grow in size or volume over time; a cancerous tumor decreases in size or volume over time; a cancerous tumor does not metastasize; or a combination of two or more of the foregoing. In embodiments, a subject responsive to immunotherapy has an increase over baseline of one or more of CD8+ cell infiltration, T cell activation, interferon-gamma pathway gene expression, T cell clone expansion, or a combination thereof, where such increase is at least 1.5-fold, or at least 2-fold, or at least 2.5-fold over baseline or a control. In embodiments, a subject responsive to immunotherapy shows a decrease (i.e., reduction) in tumor size or volume after treatment compared to baseline or a control.

The term “inhibitor,” “inhibition,” “inhibit,” “inhibiting” and the like in reference to a protein-inhibitor interaction means negatively affecting (e.g., decreasing) the activity or function of the protein relative to the activity or function of the protein in the absence of the inhibitor. In some embodiments, inhibition refers to reduction of a disease or symptoms of disease (e.g., cancer). Thus, inhibition includes, at least in part, partially or totally blocking stimulation, decreasing, preventing, or delaying activation, or inactivating, desensitizing, or down-regulating signal transduction or enzymatic activity or the amount of a protein.

“Biological sample” refers to any biological sample taken from a subject. Biological samples include blood, plasma, serum, tumors, tissue, cells, and the like. In embodiments, the biological sample is a blood sample. In embodiments, the blood sample is a peripheral blood sample. Biological samples can be taken from a subject by methods known in the art, and can be analyzed by methods known in the art.

A “control” sample or value refers to a sample that serves as a reference, usually a known reference, for comparison to a test sample. For example, a test sample can be taken from a patient suspected of having a given disease (cancer) and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. In embodiments, a control for a tumor-matching T cell is a non-tumor directed T cells (e.g., T cells directed to influenza or cytomegalovirus). One of skill will recognize that controls can be designed for assessment of any number of parameters. In embodiments, a control is a negative control. In embodiments, the control is a standard control. In embodiments, the control is a blood sample from a population of cancer subjects who are resistant or refractory to immunotherapeutic agents. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Immunotherapeutic agent” refers to therapeutic agents that activate or suppress the immune system. Exemplary immunotherapeutic agents include checkpoint inhibitors, immunomodulators, T-cell transfer therapy, cancer vaccines, and the like.

“Checkpoint inhibitor” refers to a form of immunotherapy that targets immune checkpoints, key regulators of the immune system, that when stimulated can dampen the immune response to an immunologic stimulus. Some cancers (e.g., melanoma) can protect themselves from attack by stimulating immune checkpoint targets. Checkpoint inhibitors can block inhibitory checkpoints, restoring immune system function. Exemplary checkpoint inhibitors include PD-1 inhibitors, PD-L1 inhibitors, and CTLA4 inhibitors.

“Immunomodulator” refers to therapeutic agents that modulate the immune system. Exemplary immunomodulators include interleukins (e.g., IL-2, IL-7, IL-12), cytokines (e.g., granulocyte-macrophage colony-stimulating factor, interferons, such as interferon alfa-2a, interferon alfa-2b, peginterferon alfa-2b), chemokines (e.g., CCL3, CCL26, CXCL7), immunomodulatory imide drugs (e.g., thalidomide, lenalidomide, pomalidomide, apremilast), and the like.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The terms “disease” or “condition” refer to a state of being or health status of a patient or subject capable of being treated with a compound, pharmaceutical composition, or method provided herein. In embodiments, the disease is cancer, such as lung cancer (e.g., non-small cell lung cancer), melanoma (e.g., malignant melanoma), renal cell cancer, breast cancer (e.g., triple negative breast cancer), colorectal cancer (e.g., microsatellite instable colorectal cancer), bladder cancer, prostate cancer (e.g., metastatic castration resistant prostrate cancer, castration resistant prostrate cancer), or a head and neck cancer.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer (e.g., squamous cell carcinoma of the head and neck), kidney cancer (e.g., renal cell carcinoma), myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiform, glioma, melanoma, prostate cancer, castration-resistant prostate cancer, metastatic castration resistant prostate cancer, breast cancer, triple negative breast cancer, glioblastoma, ovarian cancer, lung cancer, squamous cell carcinoma (e.g., head, neck, or esophagus), colorectal cancer (e.g., microsatellite instable colorectal cancer), leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or medulloblastoma, Hodgkin's disease, Non-Hodgkin's lymphoma, multiple myeloma, neuroblastoma, glioma, glioblastoma multiform, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, papillary thyroid cancer, hepatocellular carcinoma, Paget's disease of the nipple, phyllodes tumors, lobular carcinoma, ductal carcinoma, cancer of the pancreatic stellate cells, cancer of the hepatic stellate cells, or prostate cancer.

As used herein, the terms “metastasis,” “metastatic,” “metastatic tumor,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

A “patient” or “subject” includes both humans and other animals, particularly mammals. Thus, the methods are applicable to both human therapy and veterinary applications. In embodiments, the patient is a mammal. In embodiments, the patient is a companion animal, such as a dog or a cat. In embodiments, the patient is human.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, the term “anticancer agent” does not include immunotherapeutic agents.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic agent. In some embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib, trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin IL, or rIL-2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. paclitaxel, compounds comprising the taxane skeleton, erbulozole, dolastatin 10, mivobulin isethionate, vincristine, NSC-639829, discodermolide, ABT-751, altorhyrtins (e.g. altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE, Soblidotin, LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levami sole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

Biomarker levels may be detected at either the protein or gene expression level. Proteins expressed by biomarkers can be quantified by immunohistochemistry (IHC) or flow cytometry with an antibody that detects the proteins. Biomarker expression can be quantified by multiple platforms such as real-time polymerase chain reaction (rtPCR), Nanostring, RNAseq, or in situ hybridization. There is a range of biomarker expression across as measured by Nanostring. One skilled in the art will understand the importance of selecting a threshold of biomarker expression that constitutes increased or decreased expression of biomarkers. Controls are also valuable for determining the significance of data. For example, if values for a given parameter are widely variant in controls, variation in test samples will not be considered as significant. In some examples of the disclosed methods, when the expression level of biomarker genes is assessed, the biomarker level is compared with a control expression level of biomarker genes. By control expression level is meant the expression level of biomarker from a sample or subject lacking cancer, a sample or subject at a selected stage of cancer or cancer state, or in the absence of a particular variable such as a therapeutic agent. Alternatively, the control level comprises a known amount of biomarker genes. Such a known amount correlates with an average level of subjects lacking cancer, at a selected stage of cancer or cancer state, or in the absence of a particular variable such as a therapeutic agent. A control level also includes the expression level of biomarker genes from one or more selected samples or subjects as described herein. For example, a control level includes an assessment of the expression level of biomarker genes in a sample from a subject that does not have cancer, is at a selected stage of cancer or cancer state, or have cancer but have not yet received treatment for the cancer. Another exemplary control level includes an assessment of the expression level of biomarker genes in samples taken from multiple subjects that do not have cancer, are at a selected stage of cancer, or have cancer but have not yet received treatment for the cancer. In embodiments, the control is multiple subjects who have cancer and who are resistant or refractory to immunotherapeutic agents. In embodiments, a threshold for elevated biomarker levels is above the median expression level of a group of control sample, where the control sample is optionally a group of subjects who have cancer. In embodiments it is above the first quartile of biomarker gene expression in a group of control sample, where the control sample is optionally a group of subjects who have cancer. In embodiments it is above the third quartile of biomarker gene expression in a group of control sample.

In embodiments, quantitative rtPCR, Nanostring, RNAseq, and in situ hybridization are platforms to quantitate biomarker gene expression. For Nanostring, RNA is extracted from blood samples and a known quantity of RNA is placed on the Nanostring machine for gene expression detection using gene specific probes. The number of counts of biomarkers within a sample is determined and normalized to a set of housekeeping genes. To determine a threshold for increased or decreased biomarker levels, one skilled in the art could assess biomarker levels in a control group of samples and select the 10^{th}, 20^{th}, 25^{th}, 30^{th}, 40^{th}, 50^{th}, 60^{th}, 70^{th}, 75^{th}, 80^{th} or 90^{th} percentile of biomarker gene expression.

The increased or decreased expression of biomarkers may be determined using standard methods commonly known in the art. For example, the increased expression of biomarkers may be calculated by determining the percentage of tumor-matching T cells that are positive for the biomarkers. The tumor-matching T cells may be CD8⁺ T cells.

In embodiments, the increased or decreased expression of biomarkers may be determined by calculating the H-score for the expression of the biomarkers. Thus, the increased or decreased expression of biomarkers may have an H-score. As used herein, an “H-score” or “Histoscore” is a numerical value determined by a semi-quantitative method commonly known for immunohistochemically evaluating protein expression in tumor samples. The H-score may be calculated using the following formula: [1×(% cells 1+)+2×(% cells 2+)+3×(% cells 3+)].

According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch et al, J Clin Oncol 21: 3798-3807, 2003 and John et al, Oncogene 28:S14-S23, 2009. Immunohistochemistry or other methods known in the art may be used for detecting biomarker expression. In embodiments, the H-score of a cancer cell is determined. In embodiments, the H-score of a non-cancer cell is determined. In embodiments, the non-cancer cell is a stromal cell. In embodiments, the H-score of a cancer cell and a non-cancer cell is determined.

Methods

In embodiments, the disclosure provides methods of treating cancer in a patient in need thereof by: (i) isolating tumor-matching T cells ex vivo from blood obtained from the patient, thereby producing isolated tumor-matching T cells; (ii) expanding the isolated tumor-matching T cells ex vivo, thereby producing expanded tumor-matching T cells; and (ii) administering the expanded tumor-matching T cells to the patient, thereby treating the cancer. In embodiments, the tumor-matching T cell are CD8⁺ T cells. In embodiments, the tumor-matching T cells are functional compared to the corresponding tumor T cell. In embodiments, the tumor-matching T cells have an increased expression of a natural killer cell biomarker, an increased expression of a T cell activation marker, and a decreased expression of LTB, CCR7, GYPC, FLT3LG, or a combination of two or more thereof. In embodiments, the natural killer cell biomarker comprises KLRD1, NKG2D, KLRK1, or a combination of two or more thereof. In embodiments, the T cell activation marker comprises CXCR3, CD39, LGAS1, LGALS3, or a combination of two or more thereof. In embodiments, the tumor-matching T cells have an increased expression of KLRD1, NKG2D, KLRK1, or a combination of two or more thereof. In embodiments, the tumor-matching T cells have an increased expression of CXCR3, CD39, LGAS1, LGALS3, or a combination of two or more thereof. In embodiments, the tumor-matching T cells have an increased expression of NKG2D, CXCR3, and CD39. In embodiments, the tumor-matching T cells have an increased expression of KLRD1, NKG2D, KLRK1, CXCR3, CD39, LGAS1, LGALS3, or a combination of two or more thereof. In embodiments, the tumor-matching T cells have a decreased expression of LTB, CCR7, GYPC, FLT3LG, or a combination of two or more thereof. In embodiments, the tumor-matching T cells have an increased expression of KLRD1, NKG2D, KLRK1, or a combination of two or more thereof; an increased expression of CXCR3, CD39, LGAS1, LGALS3, or a combination of two or more thereof; and a decreased expression of LTB, CCR7, GYPC, FLT3LG, or a combination of two or more thereof. In embodiments, the tumor-matching T cells have an increased expression of at least one biomarker selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the tumor-matching T cells have an increased expression of at least two biomarkers selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG. In embodiments, the cancer is melanoma. Methods of isolating and expanding T cells ex vivo are known in the art.

In embodiments, the disclosure provides methods of treating cancer in a patient in need thereof by: (i) isolating tumor-matching T cells ex vivo from blood obtained from the patient, thereby producing isolated tumor-matching T cells; (ii) expanding the isolated tumor-matching T cells ex vivo, thereby producing expanded tumor-matching T cells; (iii) administering the expanded tumor-matching T cells to the patient; and (iv) administering to the patient an effective amount of an immunotherapeutic agent, an anti-cancer agent, or a combination thereof. In embodiments, the tumor-matching T cell are CD8⁺ T cells. In embodiments, the tumor-matching T cells are functional compared to the corresponding tumor T cell. In embodiments, the tumor-matching T cells have an increased expression of a natural killer cell biomarker, an increased expression of a T cell activation marker, and a decreased expression of LTB, CCR7, GYPC, FLT3LG, or a combination of two or more thereof. In embodiments, the natural killer cell biomarker comprises LTB, CCR7, GYPC, FLT3LG, or a combination of two or more thereof. In embodiments, the T cell activation marker comprises CXCR3, CD39, LGAS1, LGALS3, or a combination of two or more thereof. In embodiments, the tumor-matching T cells have an increased expression of KLRD1, NKG2D, KLRK1, or a combination of two or more thereof. In embodiments, the tumor-matching T cells have an increased expression of CXCR3, CD39, LGAS1, LGALS3, or a combination of two or more thereof. In embodiments, the tumor-matching T cells have an increased expression of NKG2D, CXCR3, and CD39. In embodiments, the tumor-matching T cells have an increased expression of KLRD1, NKG2D, KLRK1, CXCR3, CD39, LGAS1, LGALS3, or a combination of two or more thereof. In embodiments, the tumor-matching T cells have a decreased expression of LTB, CCR7, GYPC, FLT3LG, or a combination of two or more thereof. In embodiments, the tumor-matching T cells have an increased expression of KLRD1, NKG2D, KLRK1, or a combination of two or more thereof; an increased expression of CXCR3, CD39, LGAS1, LGALS3, or a combination of two or more thereof; and a decreased expression of LTB, CCR7, GYPC, FLT3LG, or a combination of two or more thereof. In embodiments, the tumor-matching T cells have an increased expression of at least one biomarker selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the tumor-matching T cells have an increased expression of at least two biomarkers selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG. In embodiments, the tumor-matching T cells have an increased expression of at least two biomarkers selected from the group consisting of KLRD1, NKG2D, and KLRK1; an increased expression of at least two biomarkers selected from the group consisting of CXCR3, CD39, LGAS1, and LGALS3; and a decreased expression of at least two biomarkers selected from the group consisting of LTB, CCR7, GYPC, and FLT3LG. In embodiments, the tumor-matching T cells have an increased expression of KLRD1, NKG2D, and KLRK1; an increased expression of at least three biomarkers selected from the group consisting of CXCR3, CD39, LGAS1, and LGALS3; and a decreased expression of at least three biomarkers selected from the group consisting of LTB, CCR7, GYPC, and FLT3LG. In embodiments, the tumor-matching T cells have an increased expression of KLRD1, NKG2D, and KLRK1; an increased expression of CXCR3, CD39, LGAS1, and LGALS3; and a decreased expression of LTB, CCR7, GYPC, and FLT3LG. In embodiments, the cancer is melanoma. Methods of isolating and expanding T cells ex vivo are known in the art. In embodiments, the methods comprise administering to the patient an effective amount of an anti-cancer agent. In embodiments, the methods comprise administering to the patient an effective amount of an anti-cancer agent and an immunotherapeutic agent. In embodiments, the methods comprise administering to the patient an effective amount of an immunotherapeutic agent. In embodiments, the immunotherapeutic agent is a checkpoint inhibitor. In embodiments, the immunotherapeutic agent is an immunomodulator. In embodiments, the immunotherapeutic agent is pembrolizumab, nivolumab, cemiplimab, atezolizumab, avelumab, durvalumab, or ipilimumab. In embodiments, the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, or a combination of two or more thereof.

In embodiments, the disclosure provides of treating cancer in a patient in need thereof by (i) measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, FLT3LG, or a combination of two or more thereof (ii) comparing the expression level of the one or more genes on the tumor-matching T cells to a control; (iii) identifying the patient as being responsive to immunotherapy when the expression level of one or more of LTB, CCR7, GYPC, and FLT3LG is decreased relative to the control and/or when the expression level of one or more of KLRD1, NKG2D, KLRK1, CXCR3, CD39, LGAS1, and LGALS3 is increased relative to the control; and (iv) administering to the patient who has been identified as being responsive to immunotherapy an effective amount of an immunotherapeutic agent. In embodiments, the blood sample obtained from the patient has an increased expression of KLRD1, NKG2D, KLRK1, or a combination of two or more thereof. In embodiments, the blood sample obtained from the patient has an increased expression of CXCR3, CD39, LGAS1, LGALS3, or a combination of two or more thereof. In embodiments, the blood sample obtained from the patient has an increased expression of NKG2D, CXCR3, and CD39. In embodiments, the blood sample obtained from the patient has an increased expression of KLRD1, NKG2D, KLRK1, CXCR3, CD39, LGAS1, LGALS3, or a combination of two or more thereof. In embodiments, the blood sample obtained from the patient has a decreased expression of LTB, CCR7, GYPC, FLT3LG, or a combination of two or more thereof. In embodiments, the blood sample obtained from the patient has an increased expression of KLRD1, NKG2D, KLRK1, or a combination of two or more thereof; an increased expression of CXCR3, CD39, LGAS1, LGALS3, or a combination of two or more thereof; and a decreased expression of LTB, CCR7, GYPC, FLT3LG, or a combination of two or more thereof. In embodiments, the blood sample obtained from the patient has an increased expression of at least one biomarker selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least two biomarkers selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least two biomarkers selected from the group consisting of KLRD1, NKG2D, and KLRK1; an increased expression of at least two biomarkers selected from the group consisting of CXCR3, CD39, LGAS1, and LGALS3; and a decreased expression of at least two biomarkers selected from the group consisting of LTB, CCR7, GYPC, and FLT3LG. In embodiments, the blood sample obtained from the patient has an increased expression of KLRD1, NKG2D, and KLRK1; an increased expression of at least three biomarkers selected from the group consisting of CXCR3, CD39, LGAS1, and LGALS3; and a decreased expression of at least three biomarkers selected from the group consisting of LTB, CCR7, GYPC, and FLT3LG. In embodiments, the blood sample obtained from the patient has an increased expression of KLRD1, NKG2D, and KLRK1; an increased expression of CXCR3, CD39, LGAS1, and LGALS3; and a decreased expression of LTB, CCR7, GYPC, and FLT3LG. In embodiments, the immunotherapeutic agent is a checkpoint inhibitor. In embodiments, the immunotherapeutic agent is an immunomodulator. In embodiments, the immunotherapeutic agent is pembrolizumab, nivolumab, cemiplimab, atezolizumab, avelumab, durvalumab, or ipilimumab. In embodiments, the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, or a combination of two or more thereof. In embodiments, the methods further comprise administering an effective amount of an anti-cancer agent. In embodiments, the cancer is melanoma.

In embodiments, the disclosure provides of treating cancer in a patient in need thereof by administering to the patient an effective amount of an immunotherapeutic agent, an anti-cancer agent, or a combination thereof, wherein a blood sample obtained from the patient an increased expression level of one or more of KLRD1, NKG2D, KLRK1, CXCR3, CD39, LGAS1, and LGALS3 and/or a decreased expression level of one or more of LTB, CCR7, GYPC, and FLT3LG. The increased expression level and the decreased expression level is relative to a control. In embodiments, the blood sample obtained from the patient has an increased expression of KLRD1, NKG2D, KLRK1, or a combination of two or more thereof. In embodiments, the blood sample obtained from the patient has an increased expression of CXCR3, CD39, LGAS1, LGALS3, or a combination of two or more thereof. In embodiments, the blood sample obtained from the patient has an increased expression of KLRD1, NKG2D, KLRK1, CXCR3, CD39, LGAS1, LGALS3, or a combination of two or more thereof. In embodiments, the blood sample obtained from the patient has a decreased expression of LTB, CCR7, GYPC, FLT3LG, or a combination of two or more thereof. In embodiments, the blood sample obtained from the patient has an increased expression of KLRD1, NKG2D, KLRK1, or a combination of two or more thereof; an increased expression of CXCR3, CD39, LGAS1, LGALS3, or a combination of two or more thereof; and a decreased expression of LTB, CCR7, GYPC, FLT3LG, or a combination of two or more thereof. In embodiments, the blood sample obtained from the patient has an increased expression of NKG2D, CXCR3, and CD39. In embodiments, the blood sample obtained from the patient has an increased expression of at least one biomarker selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least two biomarkers selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least two biomarkers selected from the group consisting of KLRD1, NKG2D, and KLRK1; an increased expression of at least two biomarkers selected from the group consisting of CXCR3, CD39, LGAS1, and LGALS3; and a decreased expression of at least two biomarkers selected from the group consisting of LTB, CCR7, GYPC, and FLT3LG. In embodiments, the blood sample obtained from the patient has an increased expression of KLRD1, NKG2D, and KLRK1; an increased expression of at least three biomarkers selected from the group consisting of CXCR3, CD39, LGAS1, and LGALS3; and a decreased expression of at least three biomarkers selected from the group consisting of LTB, CCR7, GYPC, and FLT3LG. In embodiments, the blood sample obtained from the patient has an increased expression of KLRD1, NKG2D, and KLRK1; an increased expression of CXCR3, CD39, LGAS1, and LGALS3; and a decreased expression of LTB, CCR7, GYPC, and FLT3LG. In embodiments, the methods comprise administering to the patient an effective amount of an anti-cancer agent. In embodiments, the methods comprise administering to the patient an effective amount of an anti-cancer agent and an immunotherapeutic agent. In embodiments, the methods comprise administering to the patient an effective amount of an immunotherapeutic agent. In embodiments, the immunotherapeutic agent is a checkpoint inhibitor. In embodiments, the immunotherapeutic agent is an immunomodulator. In embodiments, the immunotherapeutic agent is pembrolizumab, nivolumab, cemiplimab, atezolizumab, avelumab, durvalumab, or ipilimumab. In embodiments, the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, or a combination of two or more thereof. In embodiments, the cancer is melanoma.

In embodiments, the disclosure provides of treating cancer in a patient in need thereof by (i) measuring an expression level of at least one gene on tumor-matching CD8⁺ T cells in a blood sample obtained from the patient, wherein the at least one gene is selected from the group consisting of KLRD1, NKG2D, and KLRK1; (ii) measuring an expression level of at least one gene on tumor-matching CD8⁺ T cells in a blood sample obtained from the patient, wherein the at least one gene is selected from the group consisting of CXCR3, CD39, LGAS1, and LGALS3; (iii) measuring an expression level of at least one gene on tumor-matching CD8⁺ T cells in a blood sample obtained from the patient, wherein the at least one gene is selected from the group consisting of LTB, CCR7, GYPC, and FLT3LG; (iv) comparing the expression level of the genes on the tumor-matching T cells to a control; (v) identifying the patient as being responsive to immunotherapy when the expression level of one or more of LTB, CCR7, GYPC, and FLT3LG is decreased relative to the control; when the expression level of one or more of KLRD1, NKG2D, and KLRK1 is increased relative to the control; and when the expression level of one or more of CXCR3, CD39, LGAS1, and LGALS3 is increased relative to the control; and (vi) administering to the patient who has been identified as being responsive to immunotherapy an effective amount of an immunotherapeutic agent. In embodiments, the immunotherapeutic agent is a checkpoint inhibitor. In embodiments, the immunotherapeutic agent is an immunomodulator. In embodiments, the immunotherapeutic agent is pembrolizumab, nivolumab, cemiplimab, atezolizumab, avelumab, durvalumab, or ipilimumab. In embodiments, the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, or a combination of two or more thereof. In embodiments, the methods further comprise administering to the patient an effective amount of an anti-cancer agent. In embodiments, the cancer is melanoma.

In embodiments, the disclosure provides methods of identifying a cancer patient who will be responsive to immunotherapy by (i) measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, FLT3LG, or a combination of two or more thereof; (ii) comparing the expression level of the one or more genes on the tumor-matching T cells to a control; and (iii) identifying the patient as being responsive to immunotherapy when the expression level of one or more of LTB, CCR7, GYPC, and FLT3LG is decreased relative to the control and/or when the expression level of one or more of KLRD1, NKG2D, KLRK1, CXCR3, CD39, LGAS1, and LGALS3 is increased relative to the control. In embodiments, step (i) comprises measuring an expression level of NKG2D, CXCR3, and CD39. In embodiments, step (i) comprises measuring an expression level of at least two genes on tumor-matching CD8⁺ T cells in the blood sample obtained from the patient, wherein at least one gene comprises KLRD1, NKG2D, KLRK1, CXCR3, CD39, LGAS1, or LGALS3; and at least one gene comprises LTB, CCR7, GYPC, or FLT3LG. In embodiments, step (i) comprises measuring an expression level of at least three genes on tumor-matching CD8⁺ T cells in the blood sample obtained from the patient, wherein: at least two genes comprise KLRD1, NKG2D, KLRK1, CXCR3, CD39, LGAS1, or LGALS3; and at least one gene comprises LTB, CCR7, GYPC, or FLT3LG. In embodiments, step (i) comprising measuring an expression level of at least three genes on tumor-matching CD8⁺ T cells in the blood sample obtained from the patient, wherein at least one gene comprises KLRD1, NKG2D, or KLRK1; at least one gene comprises CXCR3, CD39, LGAS1, or LGALS3; and at least one gene comprises LTB, CCR7, GYPC, or FLT3LG. In embodiments, the blood sample has an increased expression of KLRD1, NKG2D, KLRK1, or a combination of two or more thereof. In embodiments, the blood sample has an increased expression of CXCR3, CD39, LGAS1, LGALS3, or a combination of two or more thereof. In embodiments, the blood sample has an increased expression of KLRD1, NKG2D, KLRK1, CXCR3, CD39, LGAS1, LGALS3, or a combination of two or more thereof. In embodiments, the blood sample has a decreased expression of LTB, CCR7, GYPC, FLT3LG, or a combination of two or more thereof. In embodiments, the blood sample has an increased expression of KLRD1, NKG2D, KLRK1, or a combination of two or more thereof; an increased expression of CXCR3, CD39, LGAS1, LGALS3, or a combination of two or more thereof; and a decreased expression of LTB, CCR7, GYPC, FLT3LG, or a combination of two or more thereof. In embodiments, the blood sample has an increased expression of at least one biomarker selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample has an increased expression of at least two biomarkers selected from the group consisting of KLRD1, NKG2D, KLRK1, CXCR3, CD39, LGAS1, and LGALS3; and a decreased expression of at least one biomarker selected from the group consisting of LTB, CCR7, GYPC, and FLT3LG. In embodiments, the blood sample has an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG. In embodiments, the blood sample obtained from the patient has an increased expression of at least two biomarkers selected from the group consisting of KLRD1, NKG2D, and KLRK1; an increased expression of at least two biomarkers selected from the group consisting of CXCR3, CD39, LGAS1, and LGALS3; and a decreased expression of at least two biomarkers selected from the group consisting of LTB, CCR7, GYPC, and FLT3LG. In embodiments, the blood sample obtained from the patient has an increased expression of KLRD1, NKG2D, and KLRK1; an increased expression of at least three biomarkers selected from the group consisting of CXCR3, CD39, LGAS1, and LGALS3; and a decreased expression of at least three biomarkers selected from the group consisting of LTB, CCR7, GYPC, and FLT3LG. In embodiments, the blood sample obtained from the patient has an increased expression of KLRD1, NKG2D, and KLRK1; an increased expression of CXCR3, CD39, LGAS1, and LGALS3; and a decreased expression of LTB, CCR7, GYPC, and FLT3LG. In embodiments, the cancer patient is a melanoma cancer patient. In embodiments, the methods further comprise (iv) administering to the patient who has been identified as being responsive to immunotherapy an effective amount of an immunotherapeutic agent. In embodiments, the immunotherapeutic agent is a checkpoint inhibitor. In embodiments, the immunotherapeutic agent is an immunomodulator. In embodiments, the immunotherapeutic agent is pembrolizumab, nivolumab, cemiplimab, atezolizumab, avelumab, durvalumab, or ipilimumab. In embodiments, the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, or a combination of two or more thereof. In embodiments, the methods further comprise administering to the patient an effective amount of an anti-cancer agent.

Compositions

The disclosure provides compositions comprising tumor-matching T cells and a growth medium. In embodiments, the tumor-matching T cell are CD8⁺ T cells. In embodiments, the tumor-matching T cells are functional compared to the corresponding tumor T cell. In embodiments, the tumor-matching T cells have an increased expression of a natural killer cell biomarker, an increased expression of a T cell activation marker, and a decreased expression of LTB, CCR7, GYPC, FLT3LG, or a combination of two or more thereof. In embodiments, the tumor-matching T cells have an increased expression of NKG2D, CXCR3, and CD39. In embodiments, the tumor-matching T cells have an increased expression of KLRD1, NKG2D, KLRK1, or a combination of two or more thereof, an increased expression of a CXCR3, CD39, LGAS1, LGALS3, or a combination of two or more thereof, and a decreased expression of LTB, CCR7, GYPC, FLT3LG, or a combination of two or more thereof. In embodiments, the tumor-matching T cells have an increased expression of at least one gene selected from the group consisting of KLRD1, NKG2D, and KLRK1, an increased expression of at least one gene selected from the group consisting of CXCR3, CD39, LGAS1, and LGALS3, and a decreased expression of at least one gene selected from the group consisting of LTB, CCR7, GYPC, and FLT3LG. In embodiments, the tumor-matching T cells have an increased expression of at least two genes selected from the group consisting of KLRD1, NKG2D, and KLRK1, an increased expression of at least two genes selected from the group consisting of CXCR3, CD39, LGAS1, and LGALS3, and a decreased expression of at least two genes selected from the group consisting of LTB, CCR7, GYPC, and FLT3LG. In embodiments, the tumor-matching T cells have an increased expression of KLRD1, NKG2D, and KLRK1, an increased expression of at least three genes selected from the group consisting of CXCR3, CD39, LGAS1, and LGALS3, and a decreased expression of at least three genes selected from the group consisting of LTB, CCR7, GYPC, and FLT3LG. In embodiments, the tumor-matching T cells have an increased expression of KLRD1, NKG2D, and KLRK1, an increased expression of CXCR3, CD39, LGAS1, and LGALS3, and a decreased expression of LTB, CCR7, GYPC, and FLT3LG. In embodiments, the growth medium comprises any growth medium known in the art that can be used to expand T cells. In embodiments, the growth medium comprises glucose, salts (e.g., sodium chloride, sodium bicarbonate, disodium phosphate, potassium chloride, magnesium sulfate, calcium nitrate), a cytokine (e.g., IL-2, IL-4, IL-7, IL-15), monoclonal antibodies (e.g., anti-CD3, anti-CD3, anti-CD28), fetal bovine serum (FBS)(e.g., 10% FBS), hydroxyethyl piperazineethane sulfonic acid (HEPES)(e.g., 1% HEPES), penicillin-streptavidin (e.g., 1% penicillin-streptavidin), amino acids (e.g., glutamine, arginine, asparagine, cysteine, leucine, isoleucine, aspartic acid), vitamins (e.g., inositol, choline chloride, folic acid, nicotinamide, biotin, riboflavin), and combinations of two or more thereof.

In embodiments, the disclosure provides containers comprising tumor-matching T cells and a growth medium. In embodiments, the tumor-matching T cell are CD8⁺ T cells. In embodiments, the tumor-matching T cells are functional compared to the corresponding tumor T cell. In embodiments, the tumor-matching T cells have an increased expression of a natural killer cell biomarker, an increased expression of a T cell activation marker, and a decreased expression of LTB, CCR7, GYPC, FLT3LG, or a combination of two or more thereof. In embodiments, the tumor-matching T cells have an increased expression of KLRD1, NKG2D, KLRK1, or a combination of two or more thereof, an increased expression of a CXCR3, CD39, LGAS1, LGALS3, or a combination of two or more thereof, and a decreased expression of LTB, CCR7, GYPC, FLT3LG, or a combination of two or more thereof. In embodiments, the tumor-matching T cells have an increased expression of NKG2D, CXCR3, and CD39. In embodiments, the tumor-matching T cells have an increased expression of at least one gene selected from the group consisting of KLRD1, NKG2D, and KLRK1, an increased expression of at least one gene selected from the group consisting of CXCR3, CD39, LGAS1, and LGALS3, and a decreased expression of at least one gene selected from the group consisting of LTB, CCR7, GYPC, and FLT3LG. In embodiments, the tumor-matching T cells have an increased expression of at least two genes selected from the group consisting of KLRD1, NKG2D, and KLRK1, an increased expression of at least two genes selected from the group consisting of CXCR3, CD39, LGAS1, and LGALS3, and a decreased expression of at least two genes selected from the group consisting of LTB, CCR7, GYPC, and FLT3LG. In embodiments, the tumor-matching T cells have an increased expression of KLRD1, NKG2D, and KLRK1, an increased expression of at least three genes selected from the group consisting of CXCR3, CD39, LGAS1, and LGALS3, and a decreased expression of at least three genes selected from the group consisting of LTB, CCR7, GYPC, and FLT3LG. In embodiments, the tumor-matching T cells have an increased expression of KLRD1, NKG2D, and KLRK1, an increased expression of CXCR3, CD39, LGAS1, and LGALS3, and a decreased expression of LTB, CCR7, GYPC, and FLT3LG. In embodiments, the growth medium comprises any growth medium known in the art that can be used to expand T cells. In embodiments, the growth medium comprises glucose, salts (e.g., sodium chloride, sodium bicarbonate, disodium phosphate, potassium chloride, magnesium sulfate, calcium nitrate), a cytokine (e.g., IL-2, IL-4, IL-7, IL-15), monoclonal antibodies (e.g., anti-CD3, anti-CD3, anti-CD28), fetal bovine serum (FBS)(e.g., 10% FBS), hydroxyethyl piperazineethane sulfonic acid (HEPES)(e.g., 1% HEPES), penicillin-streptavidin (e.g., 1% penicillin-streptavidin), amino acids (e.g., glutamine, arginine, asparagine, cysteine, leucine, isoleucine, aspartic acid), vitamins (e.g., inositol, choline chloride, folic acid, nicotinamide, biotin, riboflavin), and combinations of two or more thereof. In embodiments, the container is a petri dish, a syringe, a well plate (e.g., 96-well plate), or any other container that can be used to expand T cells.

Pharmaceutical Compositions

The disclosure provides pharmaceutical compositions comprising expanded tumor-matching T cells and a pharmaceutically acceptable excipient; wherein the tumor-matching T cells are made by a process comprising the steps of isolating tumor-matching T cells ex vivo from blood obtained from a patient, thereby producing isolated tumor-matching T cells; and expanding the isolated tumor-matching T cells ex vivo, thereby producing expanded tumor-matching T cells. In embodiments, the tumor-matching T cell are CD8⁺ T cells. In embodiments, the tumor-matching T cells are functional compared to the corresponding tumor T cell. In embodiments, the tumor-matching T cells have an increased expression of a natural killer cell biomarker, an increased expression of a T cell activation marker, and a decreased expression of LTB, CCR7, GYPC, FLT3LG, or a combination of two or more thereof. In embodiments, the tumor-matching T cells have an increased expression of KLRD1, NKG2D, KLRK1, or a combination of two or more thereof, an increased expression of a CXCR3, CD39, LGAS1, LGALS3, or a combination of two or more thereof, and a decreased expression of LTB, CCR7, GYPC, FLT3LG, or a combination of two or more thereof. In embodiments, the tumor-matching T cells have an increased expression of at least one gene selected from the group consisting of KLRD1, NKG2D, and KLRK1, an increased expression of at least one gene selected from the group consisting of CXCR3, CD39, LGAS1, and LGALS3, and a decreased expression of at least one gene selected from the group consisting of LTB, CCR7, GYPC, and FLT3LG. In embodiments, the tumor-matching T cells have an increased expression of at least two genes selected from the group consisting of KLRD1, NKG2D, and KLRK1, an increased expression of at least two genes selected from the group consisting of CXCR3, CD39, LGAS1, and LGALS3, and a decreased expression of at least two genes selected from the group consisting of LTB, CCR7, GYPC, and FLT3LG. In embodiments, the tumor-matching T cells have an increased expression of KLRD1, NKG2D, and KLRK1, an increased expression of at least three genes selected from the group consisting of CXCR3, CD39, LGAS1, and LGALS3, and a decreased expression of at least three genes selected from the group consisting of LTB, CCR7, GYPC, and FLT3LG. In embodiments, the tumor-matching T cells have an increased expression of KLRD1, NKG2D, and KLRK1, an increased expression of CXCR3, CD39, LGAS1, and LGALS3, and a decreased expression of LTB, CCR7, GYPC, and FLT3LG. In embodiments, the tumor-matching T cells have an increased expression of NKG2D, CXCR3, and CD39.

The compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

Compositions can include a single agent (expanded tumor-matching T cells or immunotherapeutic agent) or more than one agent (expanded tumor-matching T cells and an immunotherapeutic agent). The compositions for administration will commonly include an agent as described herein dissolved in a pharmaceutically acceptable carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these compositions can contain a preservative to prevent the growth of microorganisms.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The compositions of more, or highly, concentrated solutions for direct injection is also contemplated.

The formulations of compounds can be presented in unit-dose or multi-dose sealed containers, such as ampules and vials. Thus, the composition can be in unit dosage form. In such form the compositions is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

“Pharmaceutically acceptable excipient” and “pharmaceutically acceptable carrier” refer to a substance that aids the administration of an active agent to and absorption by a subject and can be included in the compositions herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such compositions can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful.

Dose and Dosing Regimens

The dosage and frequency (single or multiple doses) of the active agents (expanded tumor-matching T cells and/or immunotherapeutic agents) administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and active agents described herein. Adjustment and manipulation of established dosages (e.g., frequency and duration) are well within the ability of those skilled in the art.

For any composition and active agent described herein, the therapeutically effective amount can be initially determined from cell culture assays. Target concentrations will be those concentrations of active agents that are capable of achieving the methods described herein, as measured using the methods described herein or known in the art. As is well known in the art, effective amounts of active agents for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the active agents may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose. Thereafter, the dosage is increased by small increments until the optimum effect under circumstances is reached. Dosage amounts and intervals can be adjusted individually to provide levels of the adenosine pathway inhibitor effective for the particular clinical indication being treated. This will provide a therapeutic regimen that is commensurate with the severity of the individual's disease state.

Detection, Assay, and Diagnostic Methods

In embodiments, methods described herein may include detecting a level of, e.g., biomarkers, e.g., with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide/polynucleotide comprising a sequence that is complementary to patient genomic DNA, mRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction. Non-limiting examples of particularly useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomarkers present in the sample do not significantly bind to the binding agent specific for the target molecule. In embodiments, the level of binding to a biomolecule other than the target biomarker results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10′40⁻⁹), with high affinity antibodies in the low nanomolar (10⁻⁹) or pico molar (10⁻¹²) range for its specific target biomarker.

In embodiments, the subject matter provides a composition comprising a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, a probe on a solid support is used, and mRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In embodiments, a strip may be a nucleic acid-probe coated porous or non-porous solid support strip comprising linking a nucleic acid probe to a carrier to prepare a conjugate and immobilizing the conjugate on a porous solid support. In embodiments, the support or carrier comprises glass, polystyrene, polypropylene, polyethylene, dextran, nylon, amylases, natural and modified celluloses, polyacrylamides, gabbros, and magnetite. In embodiments, the nature of the carrier can be either soluble to some extent or insoluble for the purposes of the present subject matter. In embodiments, the support material may have any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, test strip, polystyrene beads. Those skilled in the art will know many other suitable carriers for binding antibody or antigen, or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain a biomarker binding agent alone or together with a binding agent for at least one, two, three or more other biomarkers.

In embodiments, detection is accomplished using an ELISA or Western blot format. In embodiments, the binding agent comprises an nucleic acid (e.g., a probe or primers that are complementary for mRNA or cDNA), and the detecting step is accomplished using a polymerase chain reaction (PCR) or Northern blot format, or other means of detection. In embodiments, a probe or primer is about 10-20, 15-25, 15-35, 15-25, 20-80, 50-100, or 10-100 nucleotides in length, e.g., about 10, 12, 15, 20, 25, 30, 35, 40, 45, 50, 55, 60, 70, 80, 90, or 100 nucleotides in length or less than about 30, 35, 40, 45, 50, 55, 60, 70, 80, 90, or 100 nucleotides in length.

As used herein, “assaying” means using an analytic procedure to qualitatively assess or quantitatively measure the presence or amount or the functional activity of a target entity (e.g., biomarkers as described herein). For example, assaying the level of a biomarker (such as a protein or an mRNA molecule) means using an analytic procedure (such as an in vitro procedure) to qualitatively assess or quantitatively measure the presence or amount of the compound.

In embodiments, the cells in a biological sample are lysed to release a protein or nucleic acid. Numerous methods for lysing cells and assessing protein and nucleic acid levels are known in the art. In embodiments, cells are physically lysed, such as by mechanical disruption, liquid homogenization, high frequency sound waves, freeze/thaw cycles, with a detergent, or manual grinding. Non-limiting examples of detergents include Tween 20, Triton X-100, and Sodium Dodecyl Sulfate (SDS). Non-limiting examples of assays for determining the level of a protein include HPLC, LC/MS, ELISA, immunoelectrophoresis, Western blot, immunohistochemistry, and radioimmuno assays. Non-limiting examples of assays for determining the level of an mRNA include Northern blotting, RT-PCR, RNA sequencing, and qRT-PCR.

In embodiments, once a suitable biological sample (e.g., blood) has been obtained, it is analyzed to quantitate the expression level of each of the biomarker genes. In embodiments, determining the expression level of a gene comprises detecting and quantifying RNA transcribed from that gene or a protein translated from such RNA. In embodiments, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In embodiments, when the gene expression is detected using the nCounter® Analysis System marketed by NanoString® Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In embodiments, the T-effector signature score is then calculated as the arithmetic mean of normalized values for each of the genes in the gene signature.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

### Embodiments

Embodiment 1. A method of treating cancer in a patient in need thereof, the method comprising: (i) isolating tumor-matching T cells ex vivo from blood obtained from the patient, thereby producing isolated tumor-matching T cells; (ii) expanding the isolated tumor-matching T cells ex vivo, thereby producing expanded tumor-matching T cells; and (iii) administering the expanded tumor-matching T cells to the patient, thereby treating the cancer.

Embodiment 2. The method of Embodiment 1, wherein the tumor-matching T cell are CD8⁺ T cells.

Embodiment 3. The method of Embodiment 1 or 2, wherein the tumor-matching T cells have an increased expression of at least one biomarker selected from the group consisting of KLRD1, NKG2D, KLRK1, CXCR3, CD39, LGAS1, and LGALS3.

Embodiment 4. The method of any one of Embodiments 1 to 3, wherein the tumor-matching T cells have a decreased expression of at least one biomarker selected from the group consisting of LTB, CCR7, GYPC, and FLT3LG.

Embodiment 5. The method of Embodiment 1 or 2, wherein the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG.

Embodiment 6. The method of any one of Embodiments 1 to 5, further comprising administering to the patient an effective amount of an immunotherapeutic agent, an anti-cancer agent, or a combination of thereof.

Embodiment 7. The method of Embodiment 6, wherein the immunotherapeutic agent is a checkpoint inhibitor or an immunomodulator.

Embodiment 8. The method of Embodiment 6, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof

Embodiment 9. The method of any one of Embodiments 1 to 8, wherein the cancer is melanoma.

Embodiment 10. A pharmaceutical composition comprising expanded tumor-matching T cells and a pharmaceutically acceptable excipient; wherein the tumor-matching T cells are made by a process comprising the steps of: (i) isolating tumor-matching T cells ex vivo from blood obtained from a patient, thereby producing isolated tumor-matching T cells; and (ii) expanding the isolated tumor-matching T cells ex vivo, thereby producing expanded tumor-matching T cells.

Embodiment 11. The pharmaceutical composition of Embodiment 10, wherein the tumor-matching T cell are CD8⁺ T cells.

Embodiment 12. The pharmaceutical composition of Embodiment 10 or 11, wherein the tumor-matching T cells have an increased expression of at least one biomarker selected from the group consisting of KLRD1, NKG2D, KLRK1, CXCR3, CD39, LGAS1, and LGALS3.

Embodiment 13. The pharmaceutical composition of any one of Embodiments 10 to 12, wherein the tumor-matching T cells have a decreased expression of at least one biomarker selected from the group consisting of LTB, CCR7, GYPC, and FLT3LG.

Embodiment 14. The pharmaceutical composition of Embodiment 10 or 11, wherein the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG.

Embodiment 15. A method of treating cancer in a patient in need thereof, the method comprising administering to the patient an effective amount of an immunotherapeutic agent, an anticancer agent, or a combination thereof; wherein the blood of the patient comprises a tumor-matching T cell having an increased expression level of at least one biomarker selected from the group consisting of KLRD1, NKG2D, KLRK1, CXCR3, CD39, LGAS1, and LGALS3; and/or a decreased expression level of at least one biomarker selected from the group consisting of LTB, CCR7, GYPC, and FLT3LG.

Embodiment 16. The method of Embodiment 15, wherein the tumor-matching T cell has an increased expression level of one or more of KLRD1, NKG2D, and KLRK1; and an increased expression level of one or more of CXCR3, CD39, LGAS1, and LGALS3; and a decreased expression level of one or more of LTB, CCR7, GYPC, and FLT3LG.

Embodiment 17. The method of Embodiment 15 or 16, wherein the immunotherapeutic agent is a checkpoint inhibitor or an immunomodulator.

Embodiment 18. The method of Embodiment 15 or 16, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof

Embodiment 19. A method of treating cancer in a patient in need thereof, the method comprising: (i) measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, or FLT3LG; (ii) comparing the expression level of the one or more genes on the tumor-matching T cells to a control; (iii) identifying the patient as being responsive to immunotherapy when the expression level of one or more of KLRD1, NKG2D, KLRK1, CXCR3, CD39, LGAS1, and LGALS3 is increased relative to the control; and/or when the expression level of one or more of LTB, CCR7, GYPC, and FLT3LG is decreased relative to the control; and (iv) administering to the patient who has been identified as being responsive to immunotherapy an effective amount of an immunotherapeutic agent; thereby treating the cancer in the patient.

Embodiment 20. The method of Embodiment 19, wherein the immunotherapeutic agent is a checkpoint inhibitor or an immunomodulator.

Embodiment 21. The method of Embodiment 19, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof

Embodiment 22. A method of identifying a cancer patient who will be responsive to immunotherapy, the method comprising: (i) measuring an expression level of one or more genes on tumor-matching CD8⁺ T cells in a blood sample obtained from the patient, wherein the one or more genes comprise KLRD1, NKG2D, KLRK1, CXCR3, CD39, LGAS1, LGALS3, LTB, CCR7, GYPC, FLT3LG, or a combination of two or more thereof; (ii) comparing the expression level of the one or more genes on the tumor-matching T cells to a control; and (iii) identifying the patient as being responsive to immunotherapy when the expression level of one or more of KLRD1, NKG2D, KLRK1, CXCR3, CD39, LGAS1, and LGALS3 is increased relative to the control; and/or when the expression level of one or more of LTB, CCR7, GYPC, and FLT3LG is decreased relative to the control.

Embodiment 23. The method of Embodiment 22, further administering to the cancer patient who has been identified as being responsive to immunotherapy an effective amount of an immunotherapeutic agent.

Embodiment 24. The method of Embodiment 23, wherein the immunotherapeutic agent is a checkpoint inhibitor or an immunomodulator.

Embodiment 25. The method of Embodiment 23, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof

Embodiment 26. The method of any one of Embodiments 19 to 25, where (i) comprises measuring an expression level of at least two genes on tumor-matching CD8⁺ T cells in the blood sample obtained from the patient, wherein: (a) at least one gene comprise KLRD1, NKG2D, KLRK1, CXCR3, CD39, LGAS1, or LGALS3; and (b) at least one gene comprises LTB, CCR7, GYPC, or FLT3LG.

Embodiment 27. The method of any one of Embodiments 19 to 25, where (i) comprises measuring an expression level of at least three genes on tumor-matching CD8⁺ T cells in the blood sample obtained from the patient, wherein: (a) at least two genes comprise KLRD1, NKG2D, KLRK1, CXCR3, CD39, LGAS1, or LGALS3; and (b) at least one gene comprises LTB, CCR7, GYPC, or FLT3LG.

Embodiment 28. The method of any one of Embodiments 19 to 25, where (i) comprises measuring an expression level of at least three genes on tumor-matching CD8⁺ T cells in the blood sample obtained from the patient, wherein: (a) at least one gene comprises KLRD1, NKG2D, or KLRK1; (b) at least one gene comprises CXCR3, CD39, LGAS1, or LGALS3; and (c) at least one gene comprises LTB, CCR7, GYPC, or FLT3LG.

Embodiment 29. The method of any one of Embodiments 15 to 28, wherein the cancer is melanoma.

### Examples

The following examples are for purposes of illustration only and are not intended to limit the scope of the disclosure or claims.

Here we asked whether single cell RNA sequencing could be used to track tumor-relevant T cell responses in the blood. Using the TCR as a “molecular barcode”, we utilized paired tumor and blood samples to identify and characterize tumor-matching (TM) blood CD8⁺ T cells that had shared TCR sequences with the tumor infiltrating lymphocyte (TIL) population in mice with MC38 tumors and melanoma patients. TM cells generally had an effector/effector-memory-like phenotype, and appeared less exhausted than clones in tumor. In two longitudinal samples that failed to respond to checkpoint blockade, the TM cells shifted to a stronger dysfunctional signature than before. We identified candidate surface markers that enrich for TM cells, and validated three markers using CITE-seq in mice. Importantly, combinations of these marker genes achieved improved performance compared to single markers at identifying TM cells. This work presents an approach to deeply characterize tumor-relevant T cells in blood, and identify marker panels to enable focused and statistically powered analyses of such populations.

Characterization of CD8⁺ T cells in the blood with TCRs that match to CD8⁺ T cells in MC38 tumors

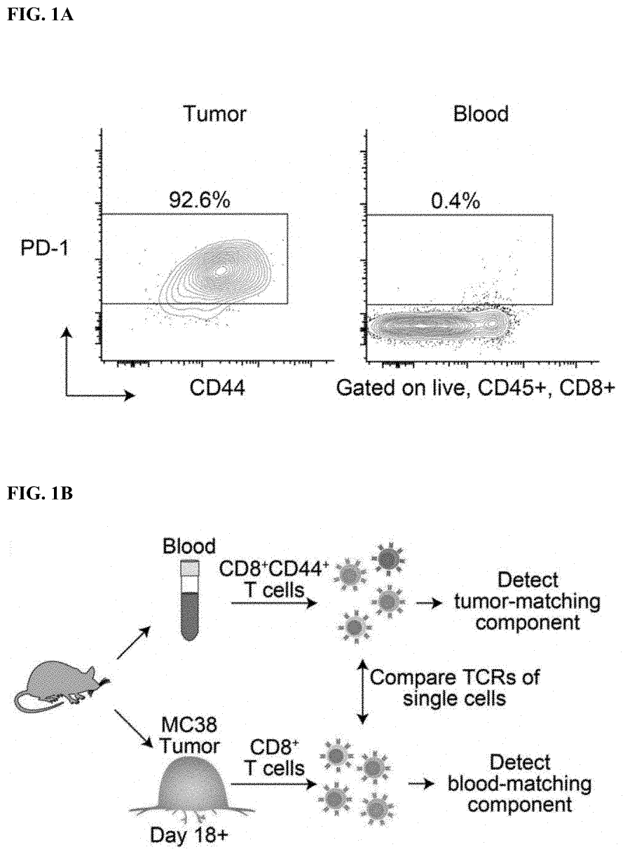

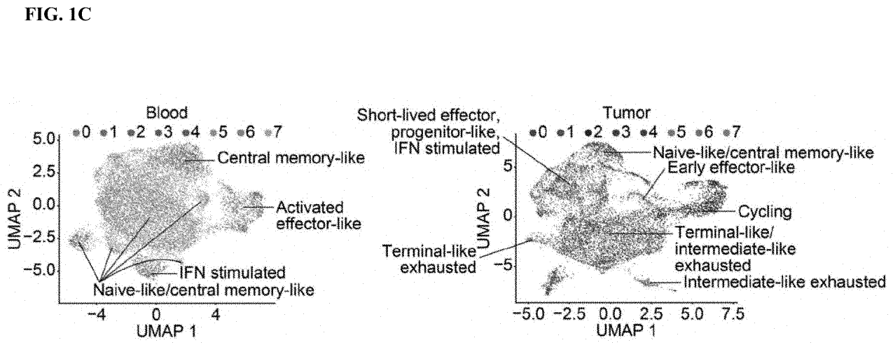

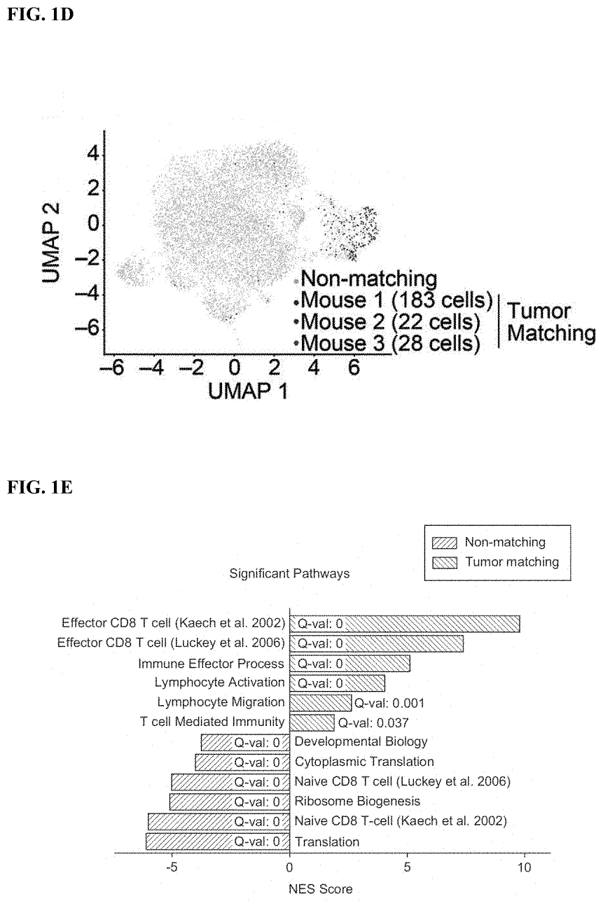

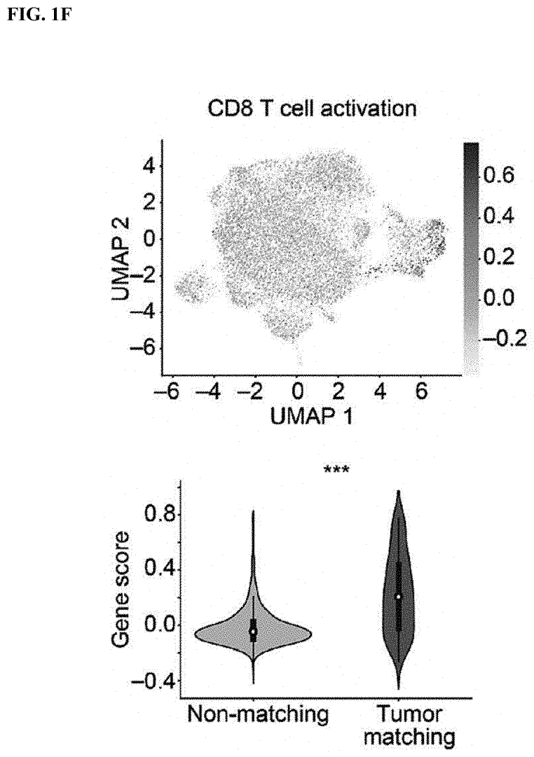

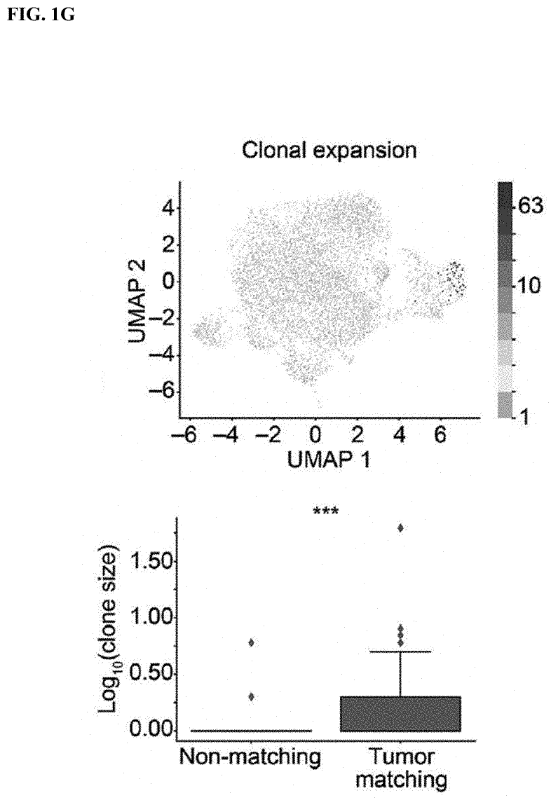

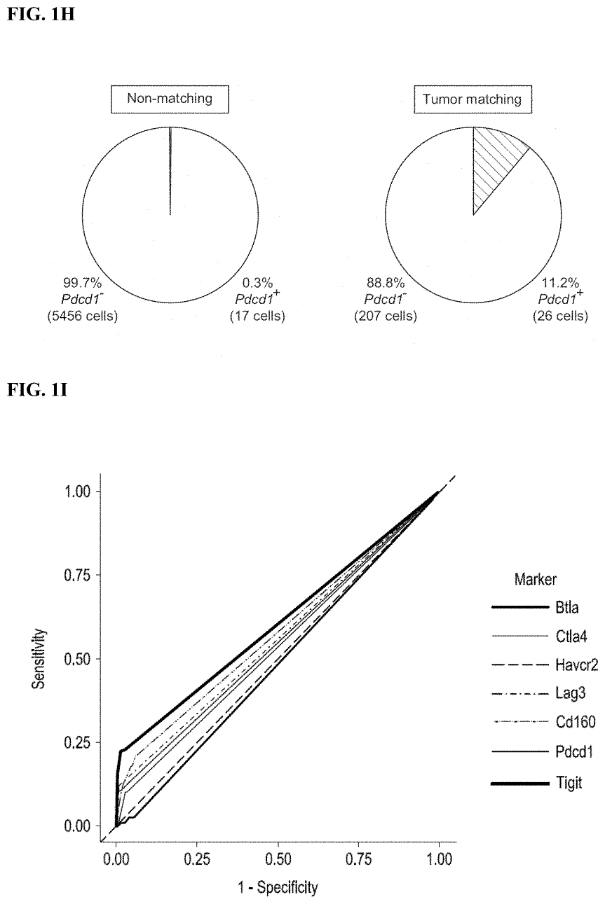

Considering the clinical relevance of tracking anti-tumor CD8⁺ T cells in the blood, we investigated ways to track these cells in tumor-bearing mice. We first assessed PD-1 protein expression on CD8⁺ T cells in mice with subcutaneous colon adenocarcinoma (MC38) tumors. PD-1 levels were uniformly high on CD8⁺ T cells in tumors, but low in the blood (FIG. 1A), casting doubt on the ability of PD-1 to capture the tumor-relevant CD8⁺ T cell component.

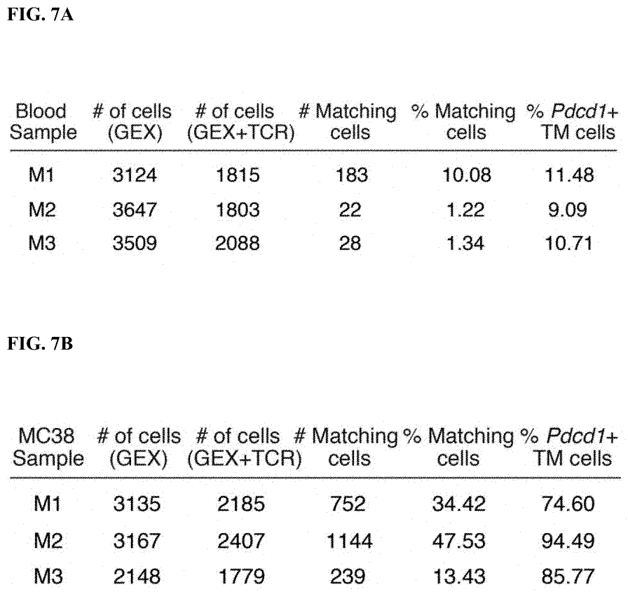

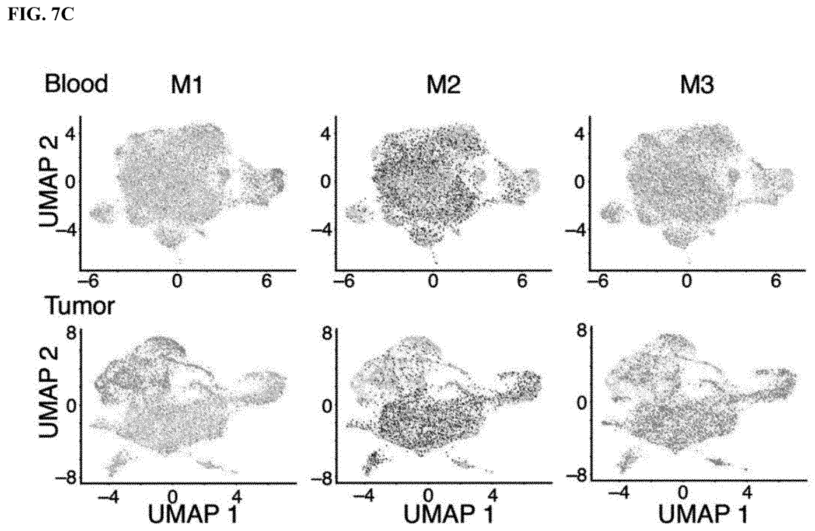

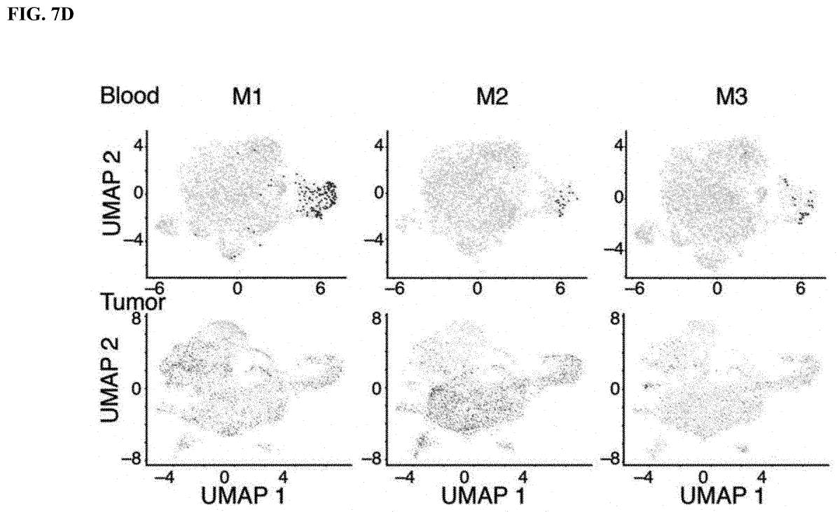

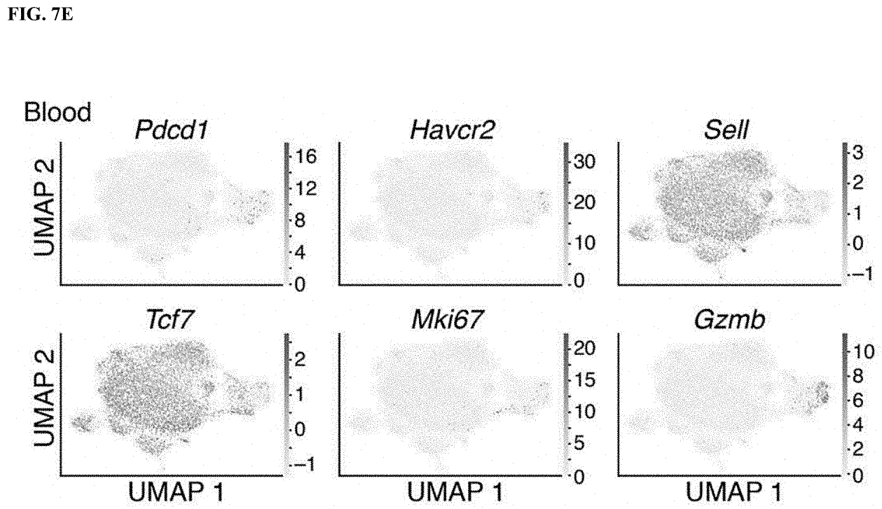

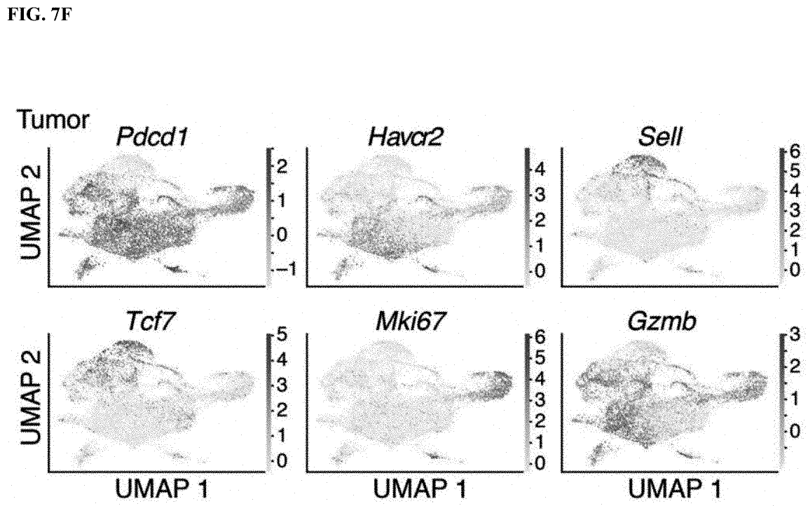

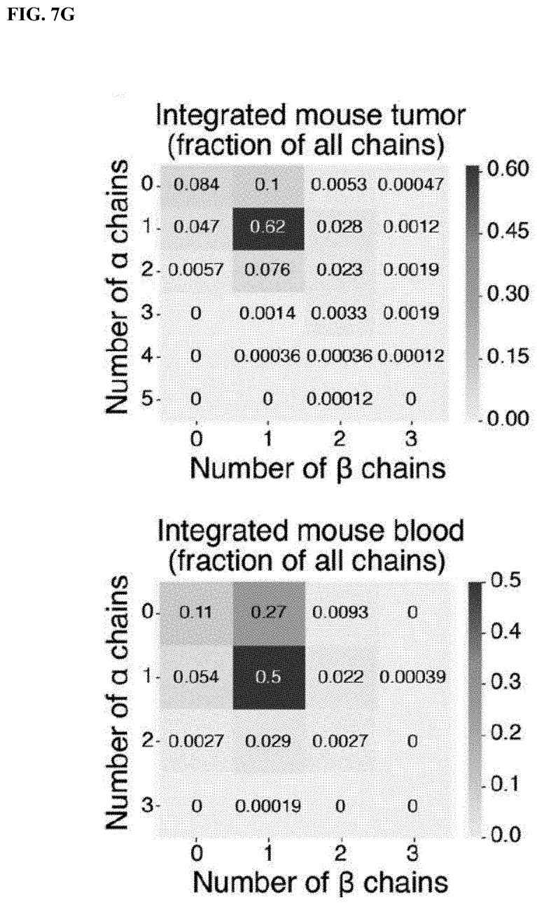

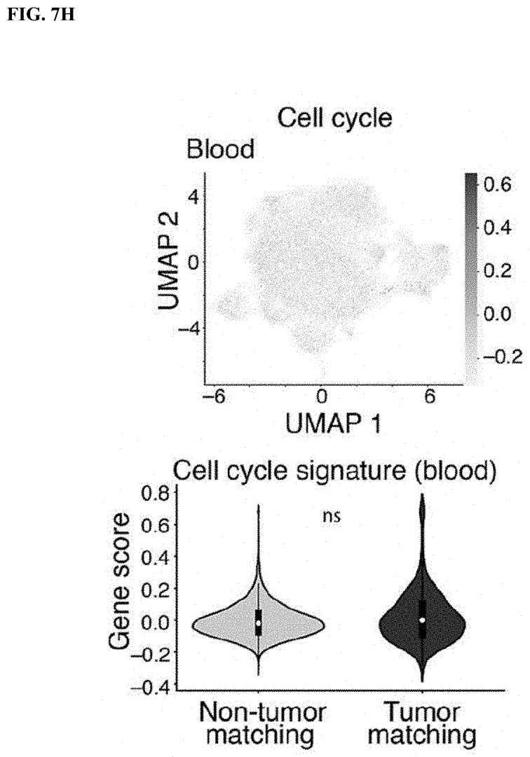

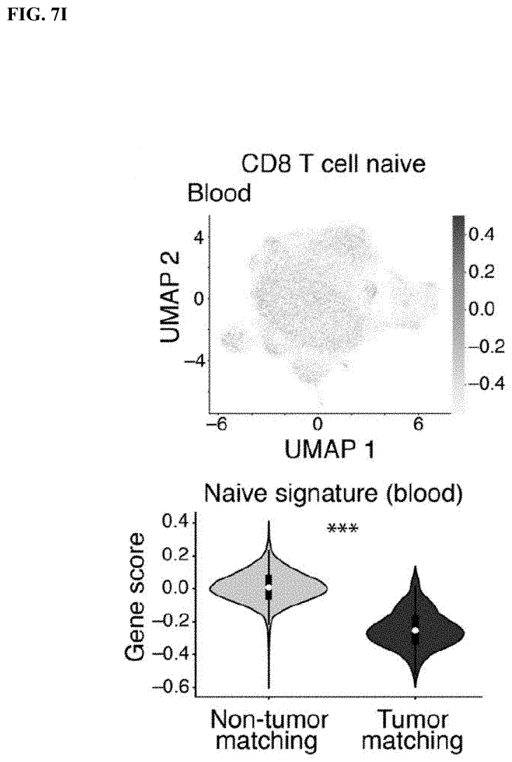

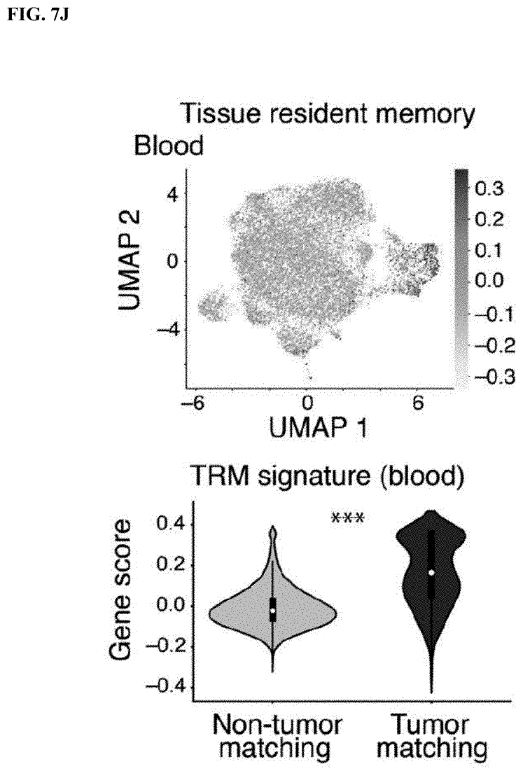

Since the TCR encodes specificity for antigen, we hypothesized that the TCR sequence could be used to assess which clones in blood were relevant to the anti-tumor response. To test this, we performed single-cell RNA and TCR sequencing on CD8⁺ T cells isolated from paired blood and MC38 tumors (FIGS. 1B, 7A-7F). The single cell transcriptomic landscapes of sorted CD44⁺CD8⁺ T cells in blood (n=10,289 cells) (to enrich for rare antigen-experienced cells) and bulk CD8⁺ T cells in tumors (n=8,540 cells) were characterized (FIGS. 1C, 7E-7F). In the blood, most of the cells had a naïve-like and/or central-memory like phenotype (FIG. 1C, Table S1) as expected in specific pathogen-free mice (Beura et al., 2016). Additional phenotypes included recent interferon (IFN) stimulation and an activated effector-like population (FIG. 1C, Table S1). In the tumor, more diversity was observed, including progenitor and terminal exhausted subsets (He et al., 2016; Im et al., 2016; Kurtulus et al., 2019; Miller et al., 2019; Sade-Feldman et al., 2018; Siddiqui et al., 2019; van der Leun et al., 2020), as well as an intermediate-like exhausted subset, naïve and/or central memory-like cells, effector-like cells, cycling cells, and IFN-stimulated cells (FIG. 1C, Table S1) (Best et al., 2013; Kakaradov et al., 2017; Milner et al., 2017). These data highlight the diversity of CD8⁺ T cell states in MC38 tumors, particularly compared to states present in blood (FIG. 1C, Table S1).

To assess clonal overlap between blood and tumor, we (a) compared T cells with at least one alpha and one beta chain (FIG. 7G), and (b) classified cells as the same clone if they exactly matched in their TCR sequences. Using the TCR sequence as a molecular barcode, we observed a population of “tumor matching” (TM) cells in blood that shared TCRs with CD8⁺ T cells in the tumor (FIGS. 1D, 7A, 7D). Differential gene (DE) analysis showed elevated activation markers (e.g. Ccl5, Gzmb, Klrg1, Itgal, Klrk1, and Cx3cr1) and decreased naïve-like and/or central memory-like markers (e.g. Ccr7, Sell, Il7r, and Tcf7) in TM cells compared to non-tumor matching (non-TM) cells (Table S2). Pathway enrichment analysis of genes in TM cells showed effector signatures, immune effector processes, and lymphocyte migration, while non-TM cells were enriched for naïve CD8⁺ T cell signatures (FIG. 1E, Table S3). Additionally, using curated signatures from the literature (see Methods), TM cells were enriched for activation and tissue resident memory (TRM) signatures, while non-TM cells were enriched for a naïve signature (FIGS. 1F, 7I-7J, Table S3). TM cells were also more likely to be clonally expanded (FIG. 1G), though a signature of cell cycle was low (FIG. 7H). Importantly, only 11.2% of TM cells expressed the Pdcd1 transcript (FIG. 1H). Using receiver operating characteristic (ROC) curves, Pdcd1 and other inhibitory receptors performed poorly in distinguishing TM cells from non-TM cells, nearing the level of random chance (FIG. 1I). Collectively, these data are consistent with TM cells actively responding to tumor, and support using the TCR to identify TM cells rather than relying on individual markers like PD-1.

The transcriptional signature of tumor-matching CD8⁺ T cells in the blood can be used to identify markers for enrichment via flow cytometry

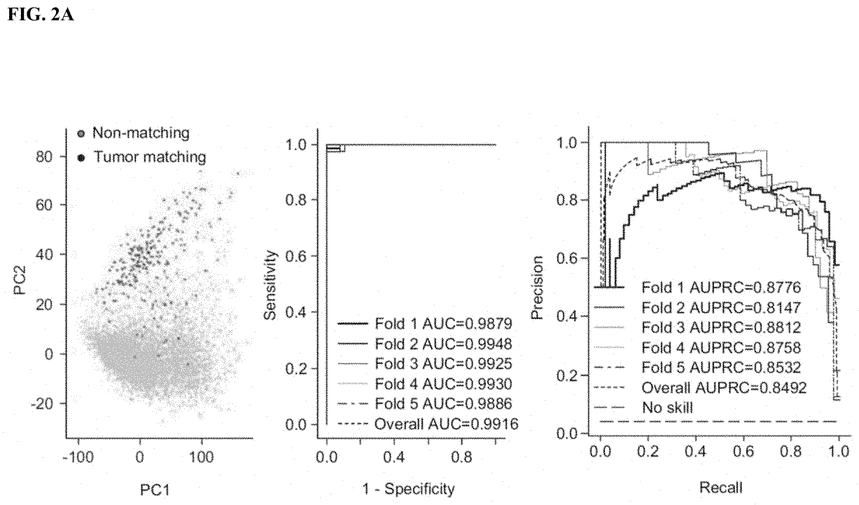

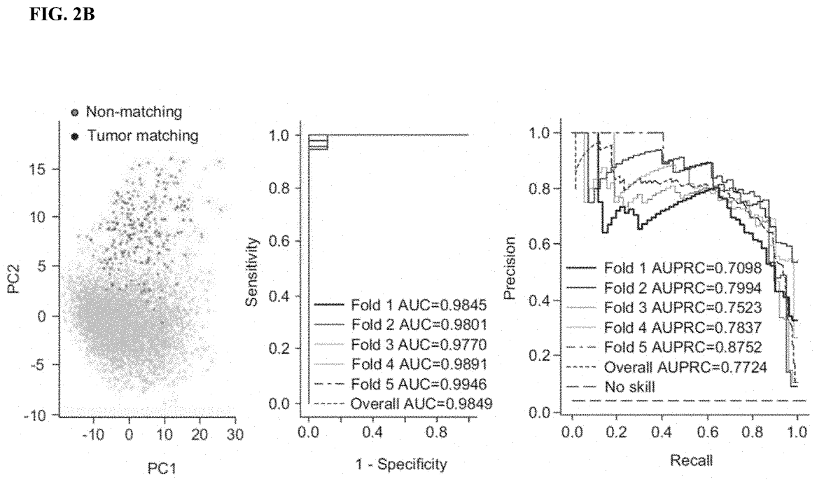

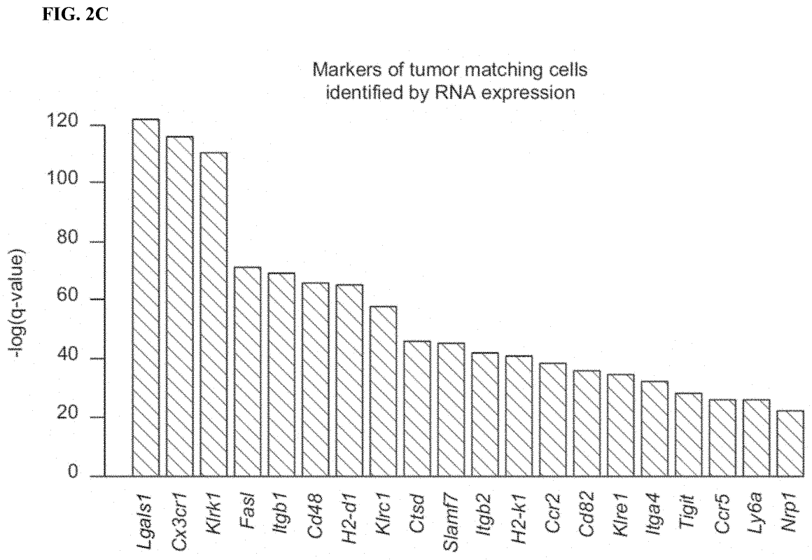

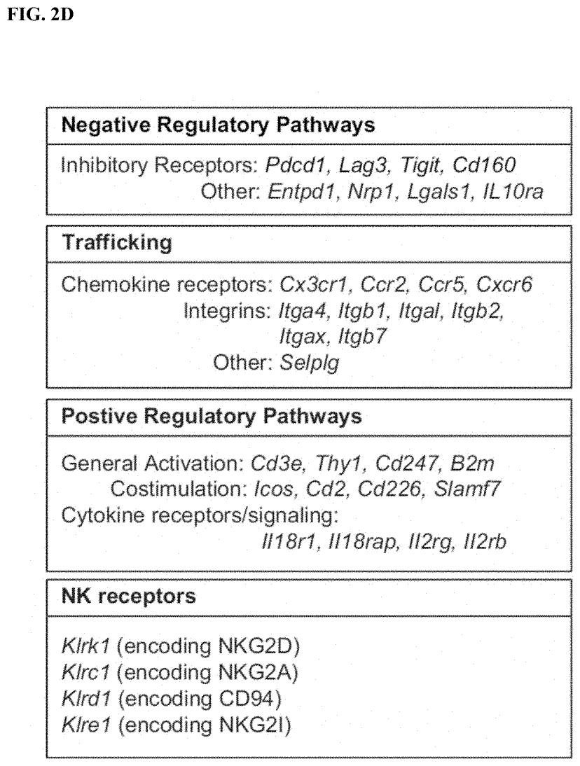

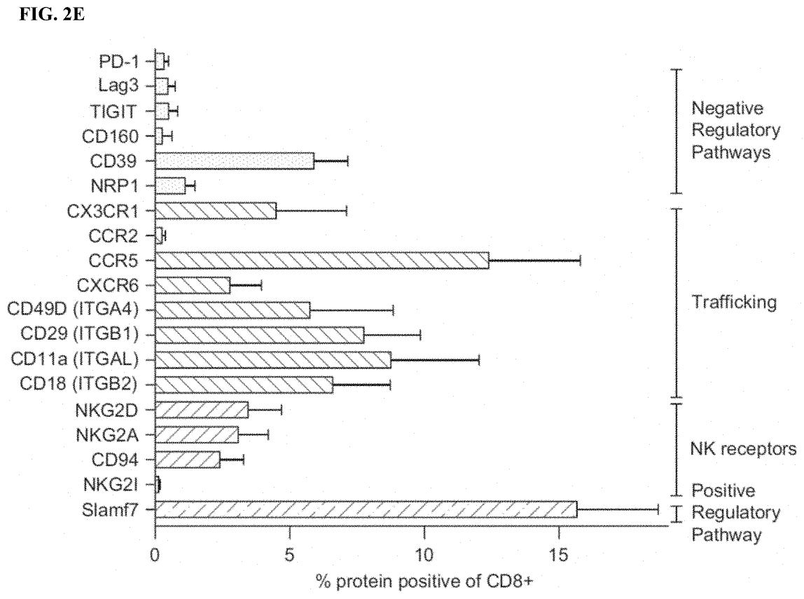

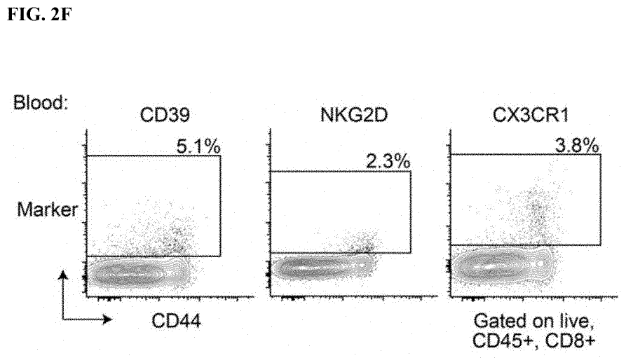

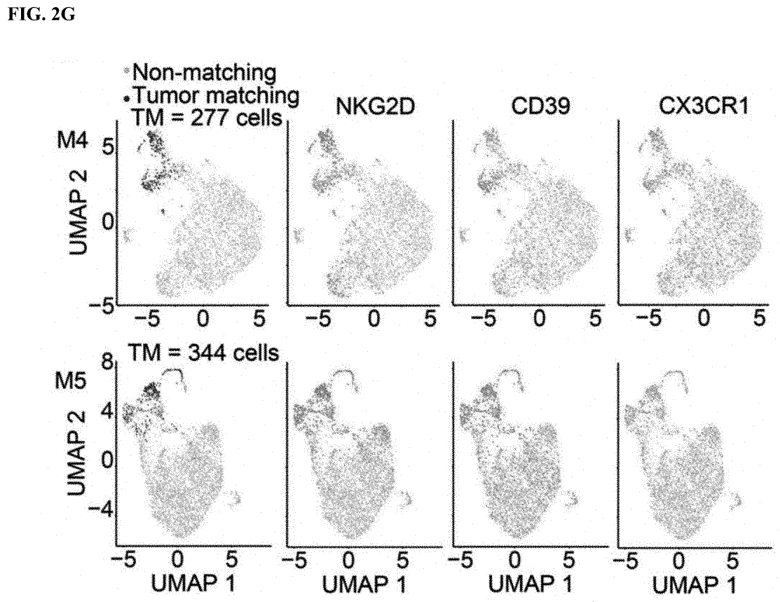

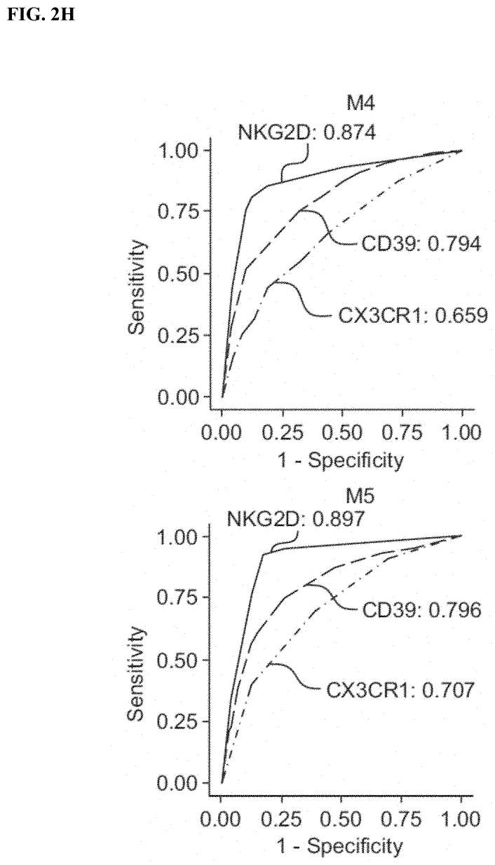

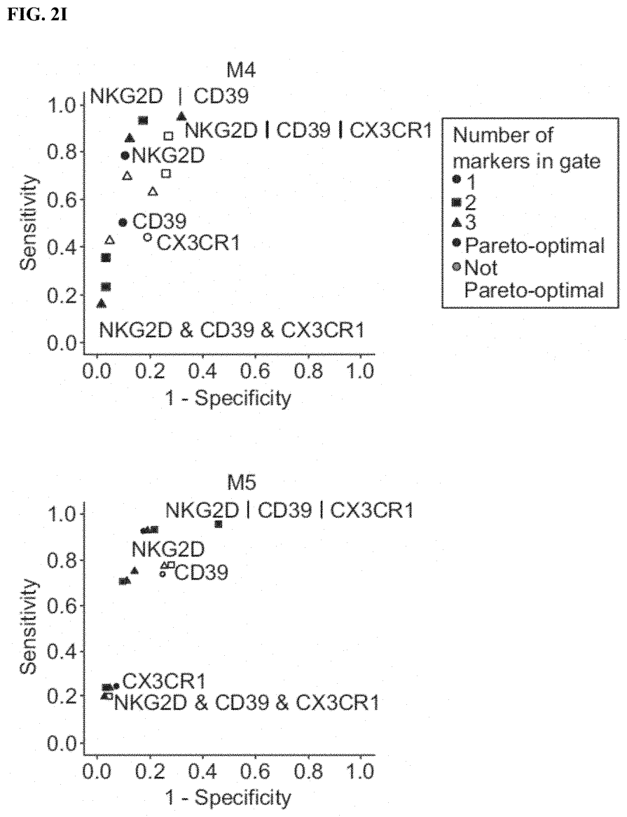

Following our observation that TM cells are transcriptionally distinct, we hypothesized that a machine learning classifier could be trained to predict if a given CD8⁺ T cell from blood is TM or non-TM based on transcriptional data. Indeed, a regularized logistic regression classifier achieved high sensitivity and specificity (FIG. 2A, cross validated AUC=0.99). We next asked if cell surface genes would be sufficient to distinguish TM from non-TM cells, to assess the potential of identifying cell surface marker panels for flow cytometry-based sorting for downstream applications. Classifiers utilizing only a list of cell-surface genes (Chihara et al., 2018) also achieved high sensitivity and specificity (FIG. 2B, cross validated AUC=0.985).

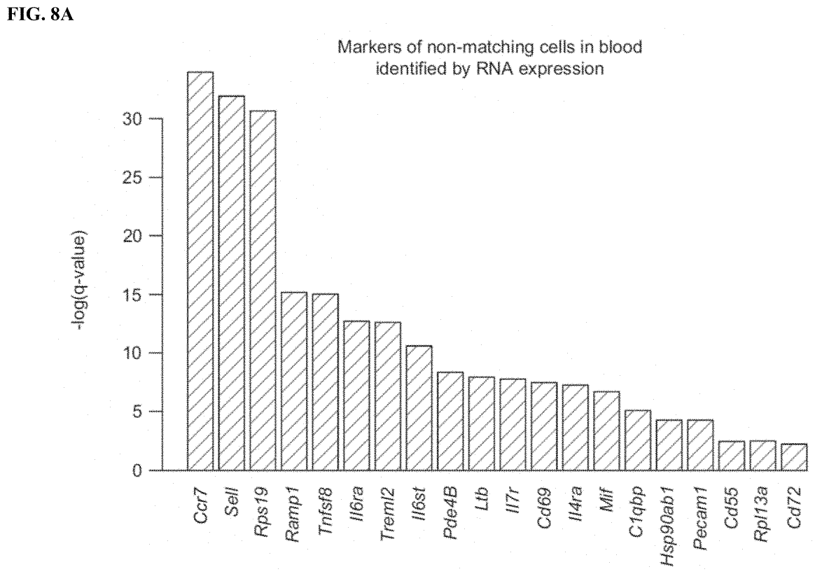

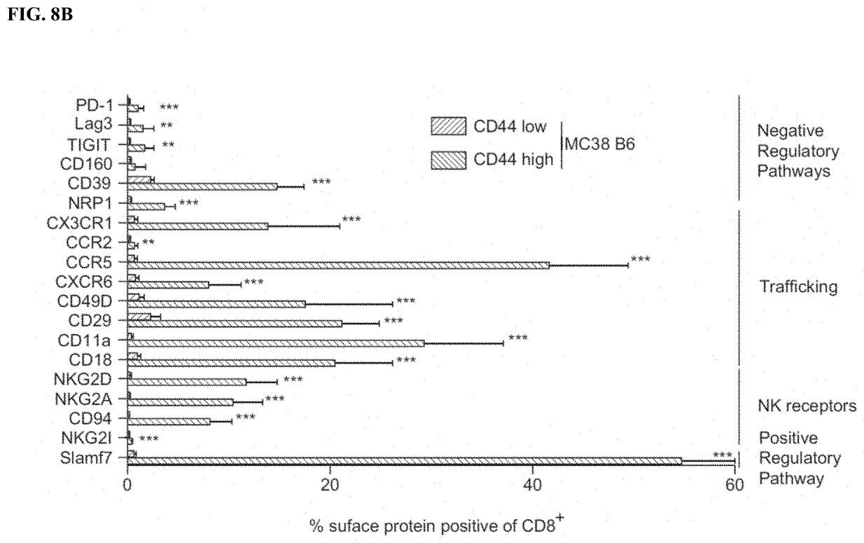

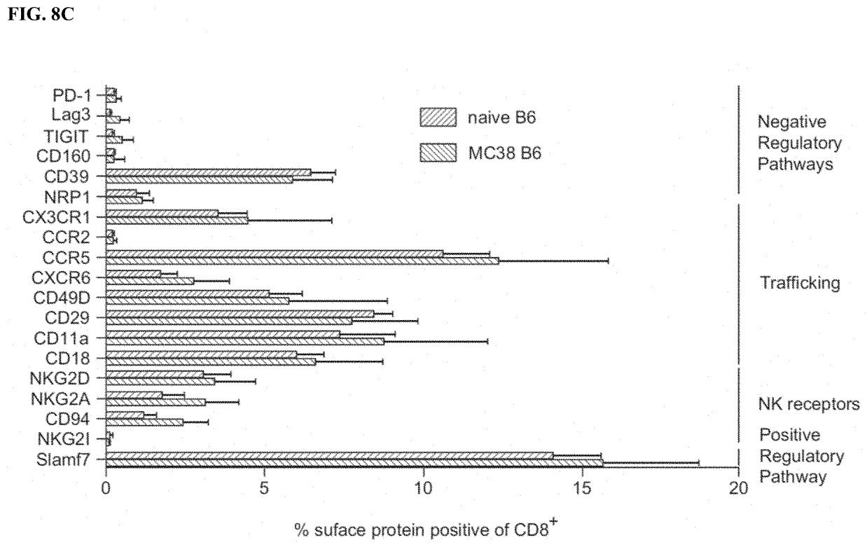

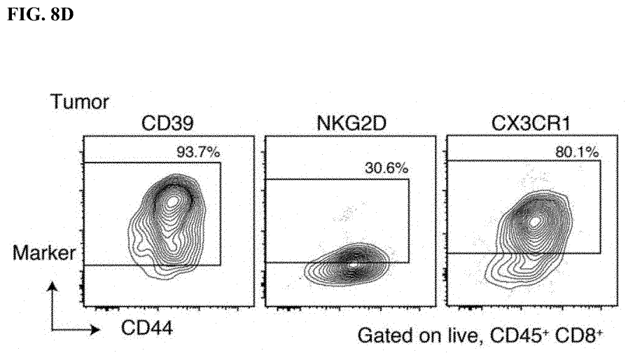

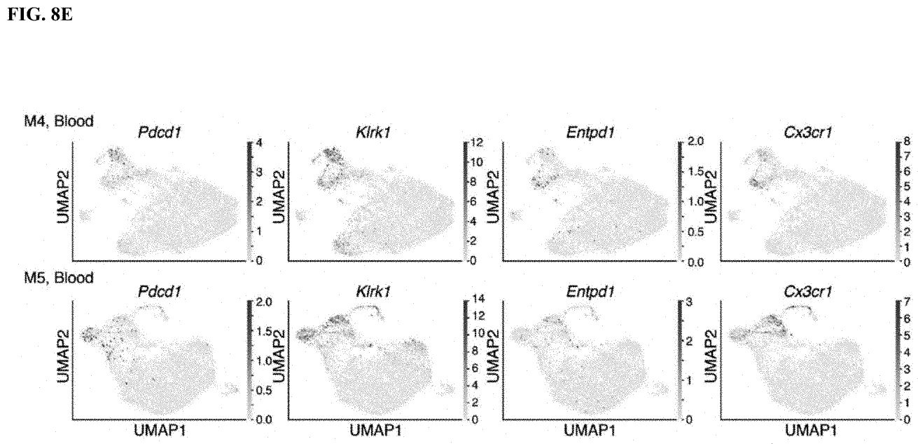

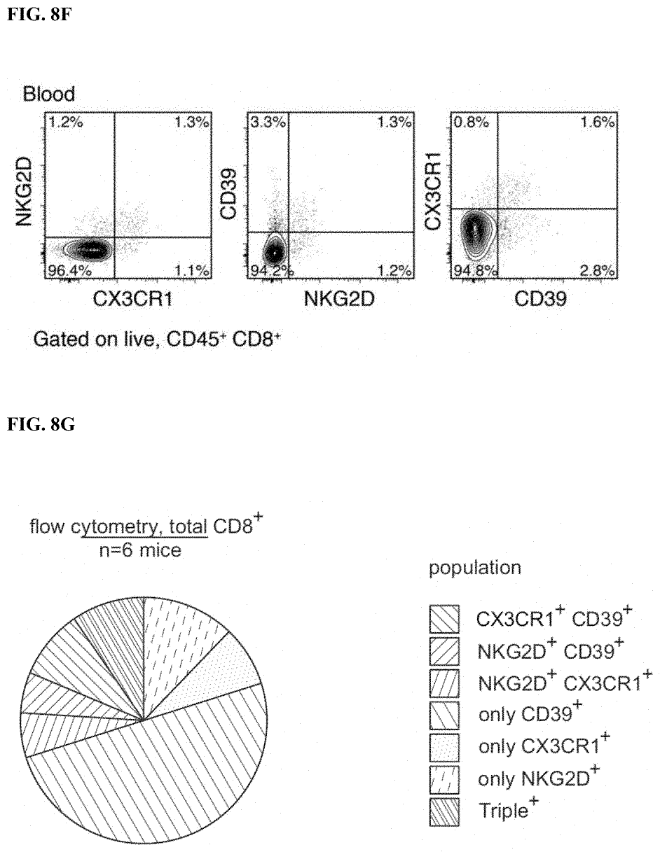

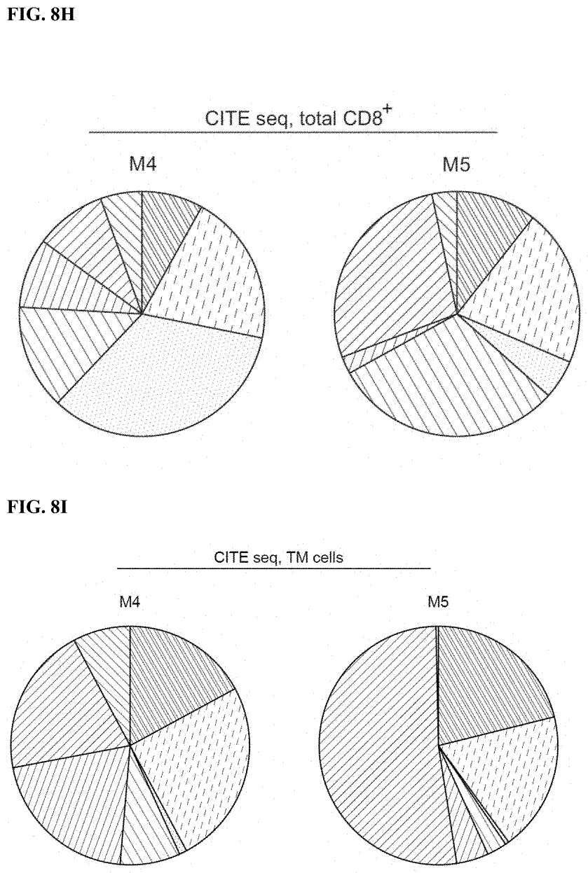

To test whether single-gene surface markers could identify the TM component we applied COMET, a computational tool we developed to predict markers from single-cell RNA-seq data (Delaney et al., 2019). COMET identified 82 candidate positive markers for the TM component (with q value<=0.01) classified into four general biological categories: negative regulatory pathways, positive regulatory pathways (including general activation, costimulation, and cytokine receptors/signaling), trafficking molecules, and NK receptors (FIGS. 2C-2D, Table S4). COMET also identified 21 candidate positive markers associated with non-TM cells (FIG. 8A), many consistent with their naïve and/or central memory-like phenotype (e.g. Ccr7, Sell, Il7r) (FIGS. 1E, 7I).

Several candidate markers were also detected at the protein level (FIG. 2E), and enriched on CD44⁺ cells (FIG. 8B). Some markers trended towards a higher frequency in the blood of mice bearing MC38 tumors than naïve mice, but many including PD-1 were not different (FIG. 8C). To test if surface proteins could enrich for TM cells, we evaluated three of the COMET-predicted candidates (Entpd1 encoding CD39, Cx3cr1 encoding CX3CR1, and Klrk1 encoding NKG2D) (FIG. 2D, Table S4). A small number of CD8⁺ T cells expressed these proteins in the blood of mice with MC38 tumors (FIGS. 2E-2F), albeit less than observed in the tumor (FIG. 8D). We next performed a single-cell experiment measuring gene expression, TCR, and protein expression for CD39, CX3CR1, and NKG2D using CITE-seq (Stoeckius et al., 2017) in two mice (FIGS. 2G, 8E) to determine if these proteins could enrich for TM cells identified using the TCR. As single markers, each protein successfully enriched for TM cells (FIGS. 2G-2H). We next asked if combinations were useful for identifying TM cells. Most TM cells expressed two or three of the markers (FIGS. 8F-8I). Moreover, using combinations improved on either or both the sensitivity and specificity over single markers (FIG. 2I, Table S5). Consequently, while the TCR likely remains the most sensitive and specific metric for determining whether T cells have shared reactivity, cell surface markers can be identified and used to distinguish TM cells from non-TM cells.

Tumor-matching CD8⁺ T cells in blood are less dysfunctional than matching clones found in the tumor

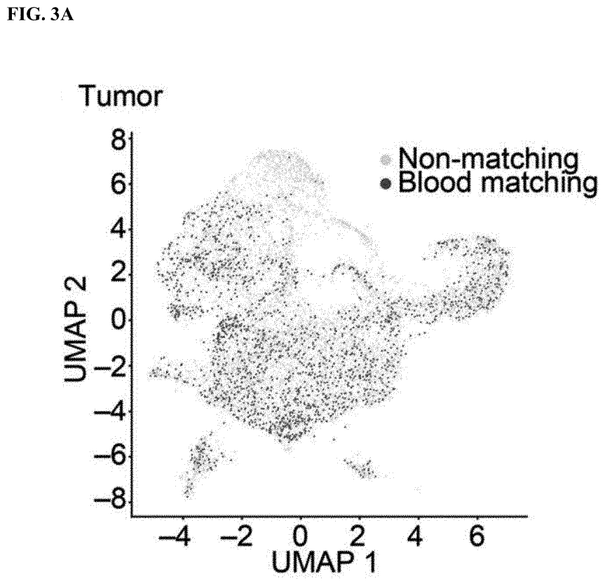

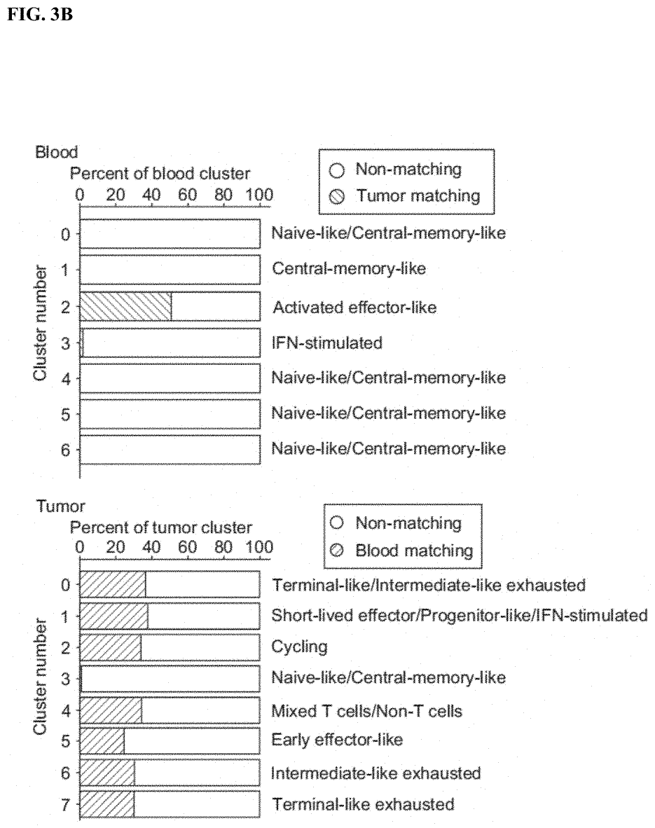

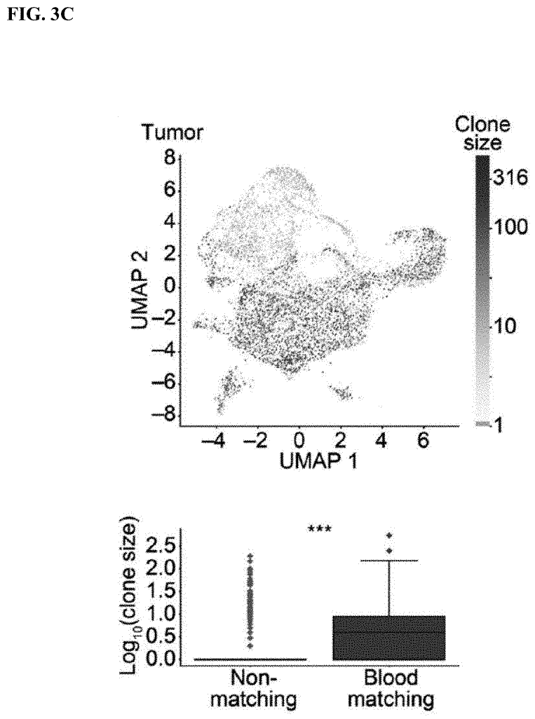

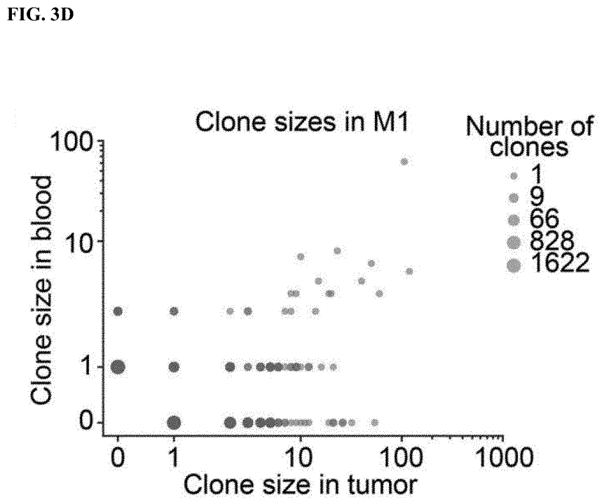

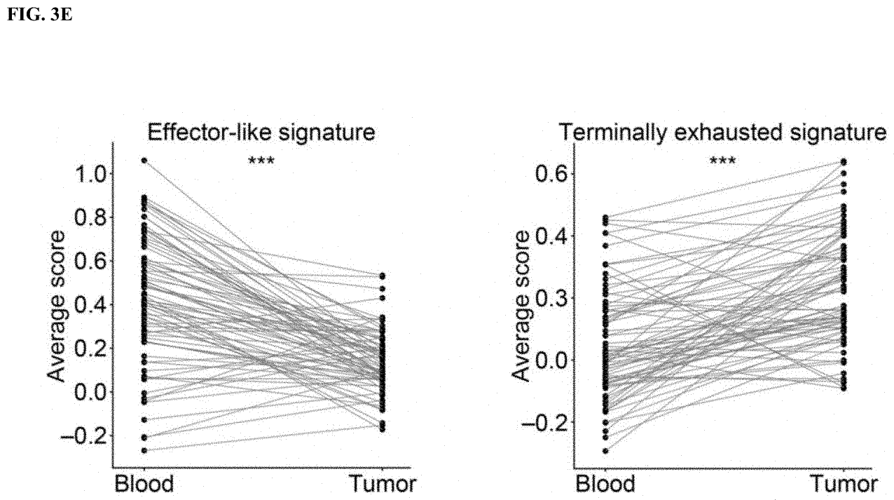

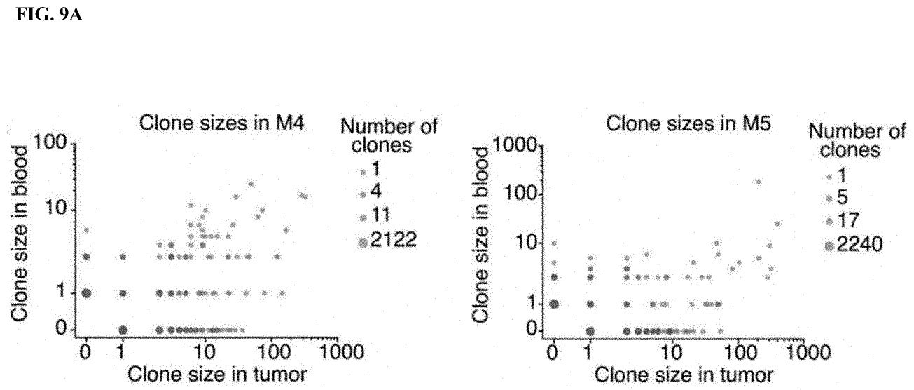

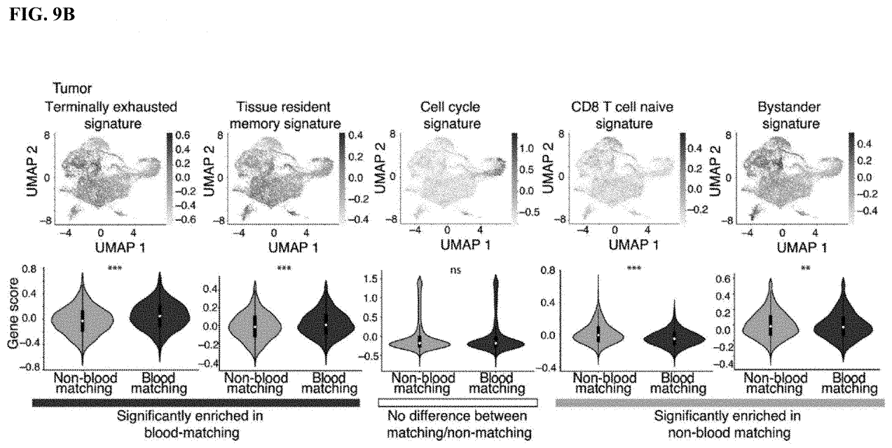

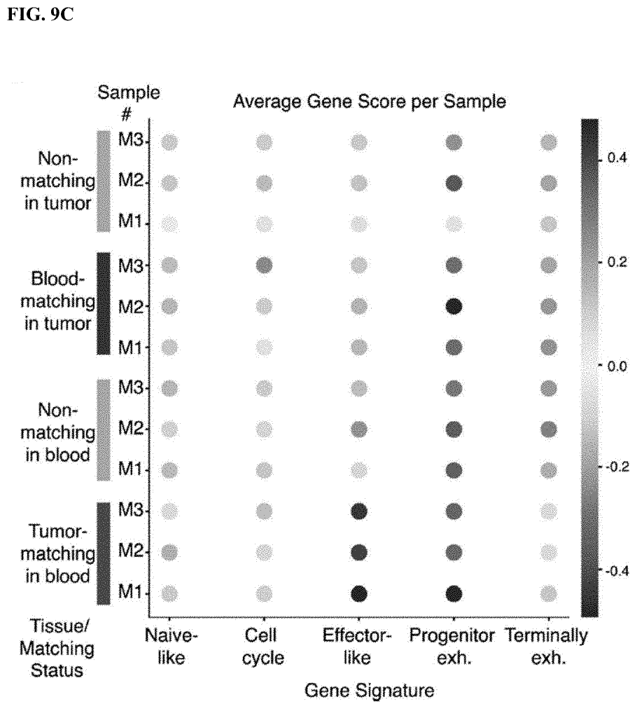

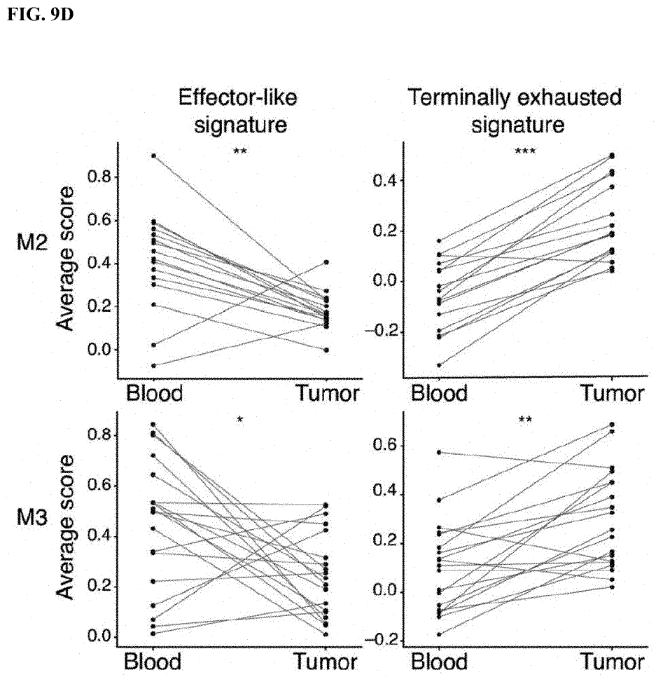

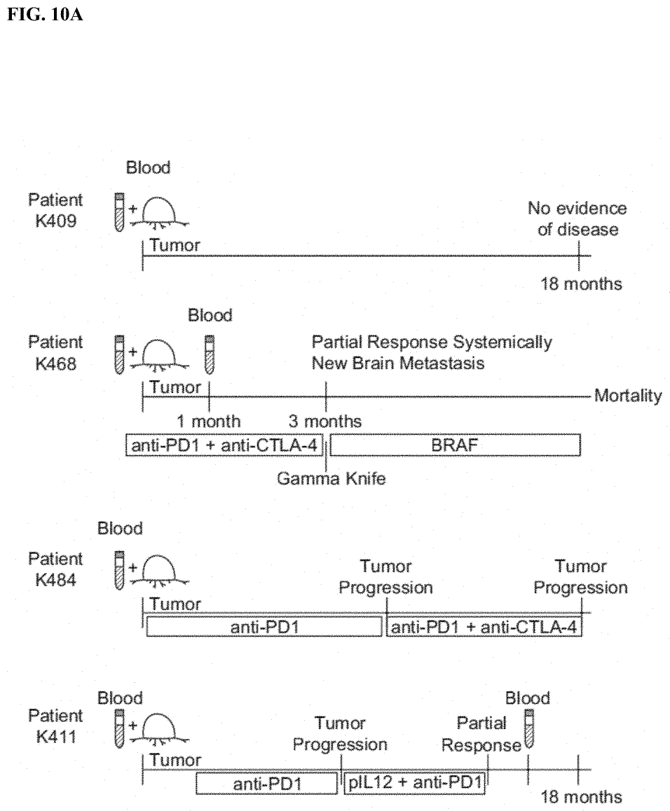

We next examined the transcriptional heterogeneity of CD8⁺ T cells in the tumor whose TCRs were also detected in blood, referred to as “blood-matching” cells. Blood-matching cells were present in every transcriptional cluster in the tumor (FIGS. 3A-3B, 7D), with the majority present in non-naïve/central memory-like clusters in the tumor (FIG. 3B). Blood-matching cells were more clonally expanded than non-matching cells (FIG. 3C, p=4.9×10⁻²⁶). In Mouse 1, we observed a correlation between clone size in blood and clone size in tumor (FIG. 3D). While clone sizes were too low in Mouse 2 and Mouse 3 to observe a significant correlation in expansion between blood and tumor, we did observe this correlation in the two mice in our validation cohort (Mouse 4 and Mouse 5) where the number of TM cells recovered was higher (FIG. 9A).

To further characterize blood-matching T cells in the tumor, we examined signatures related to CD8⁺ T cell functions. Compared to non-matching cells, blood-matching cells expressed higher levels of a terminal exhaustion signature and a TRM signature, associated with TRM cells which play a role in protective anti-tumor immunity (Menares et al., 2019; Park et al., 2019) (FIG. 9B). Blood-matching cells expressed lower levels of a naïve T cell signature, and no difference was observed in a cell-cycle signature (FIG. 9B). Lastly, pathogen-specific CD8⁺ T cells can infiltrate tumors in both mice and humans (Mognol et al., 2017; Rosato et al., 2019; Simoni et al., 2018). This bystander transcriptional signature (Mognol et al., 2017) was observed in MC38 tumors (FIG. 9B), but expressed at lower levels in blood-matching cells compared to non-matching cells. These findings indicate that the TM component in blood corresponds to matching clones in the tumor that are likely responding to tumor antigens and relevant for tumor killing.

Next, we compared the transcriptional profiles of TM cells in blood to matching clones in the tumor. The blood-matching population within tumor was more diverse than the TM population in blood (FIG. 3B), indicating that CD8⁺ T cells can diversify and take on a number of states upon entering tumors. Both on a population level (FIG. 9C) and on a clone by clone basis (FIGS. 3E, 9D), TM cells were significantly more enriched for an effector-like signature than blood-matching cells in tumor, and blood-matching cells in the tumor were more enriched for the terminal exhaustion signature than TM cells in blood (FIGS. 3E, 9D). Additionally, DE gene analysis on clonally matched populations between blood and tumor showed many effector-like genes up regulated in TM clones in the blood (e.g. Ccl5, Cx3cr1, Itga4, Runx1, and Klrg1), and exhausted-like genes up-regulated in the blood-matching clones in tumor (e.g. Pdcd1, Lag3, Ctla4, Havcr2, Tigit) (Table S6). Clones in tumors also showed elevated levels of many of the granzymes (Gzmb, Gzmc, Gzmf, Gzmg) (Table S6), consistent with work showing some overlap between effector-associated genes and exhausted T cell populations, particularly terminally exhausted T cells (Beltra et al., 2020; Singer et al., 2016). These data indicate that TM cells in the blood are less dysfunctional than their counterparts in tumor, and that after migration into the tumor these TM cells acquire a dysfunctional state.

Activated tumor-matching CD8⁺ T cells can be detected in the blood of melanoma patients

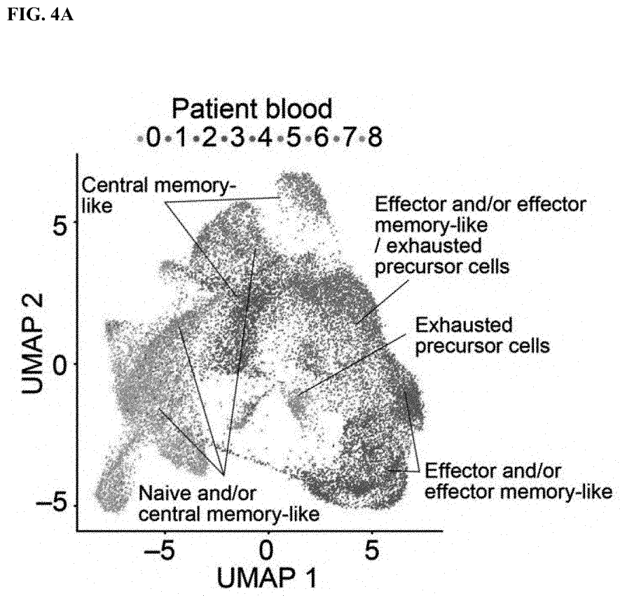

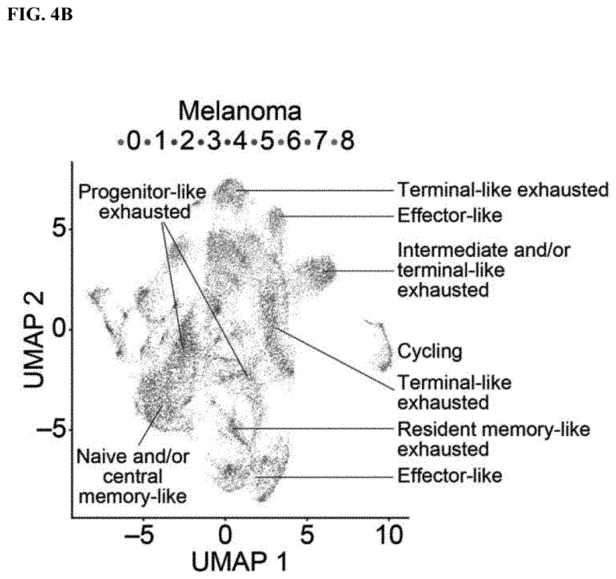

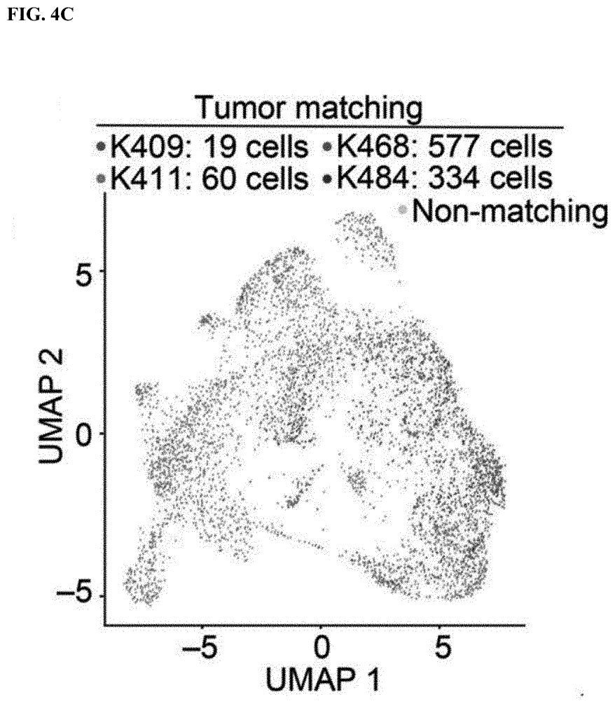

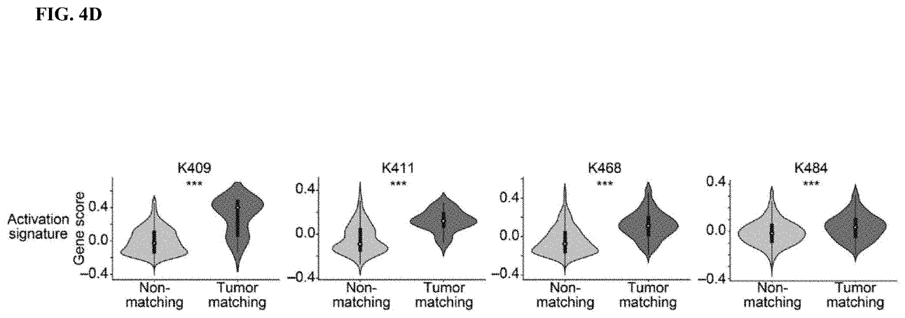

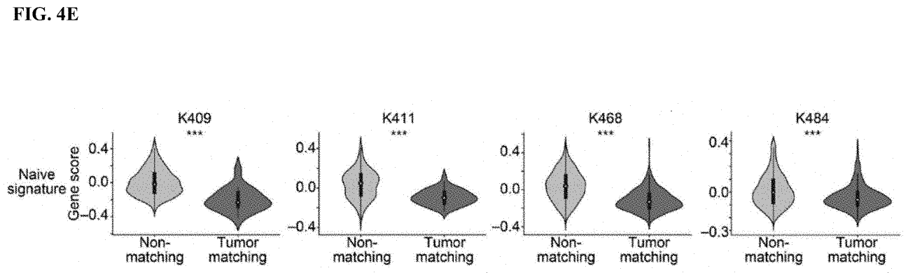

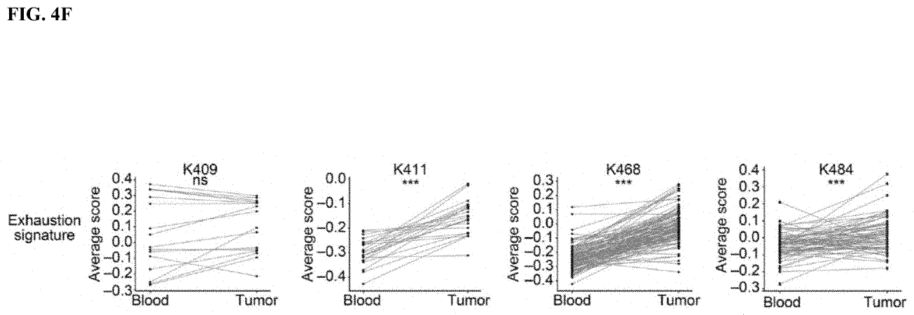

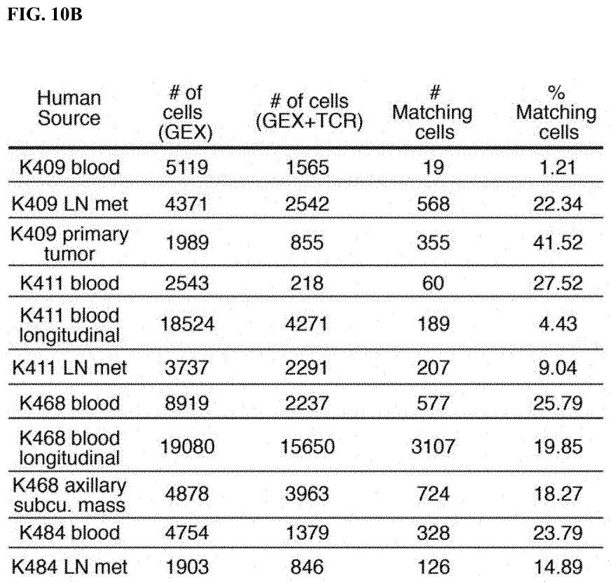

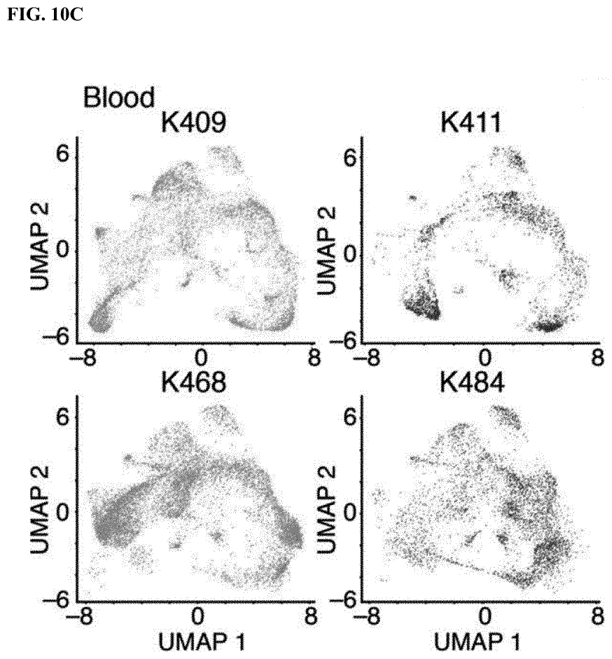

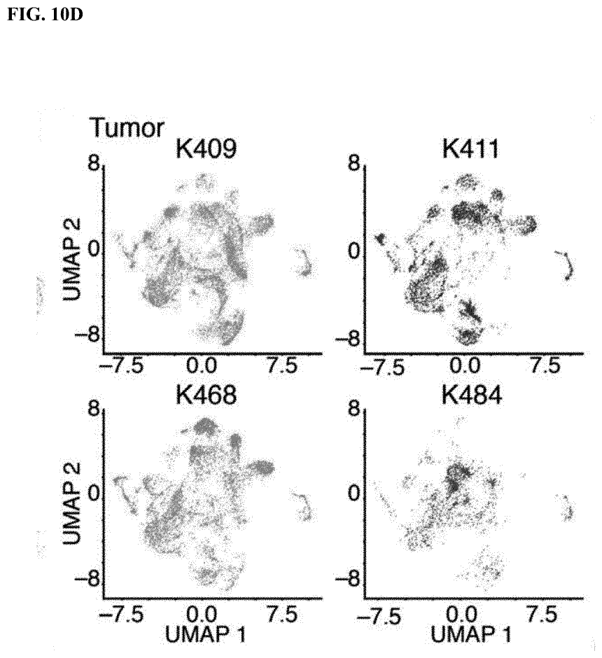

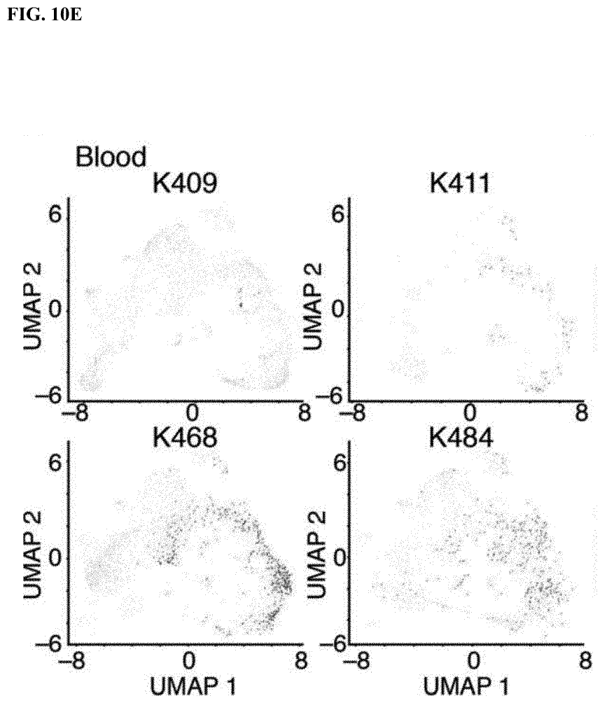

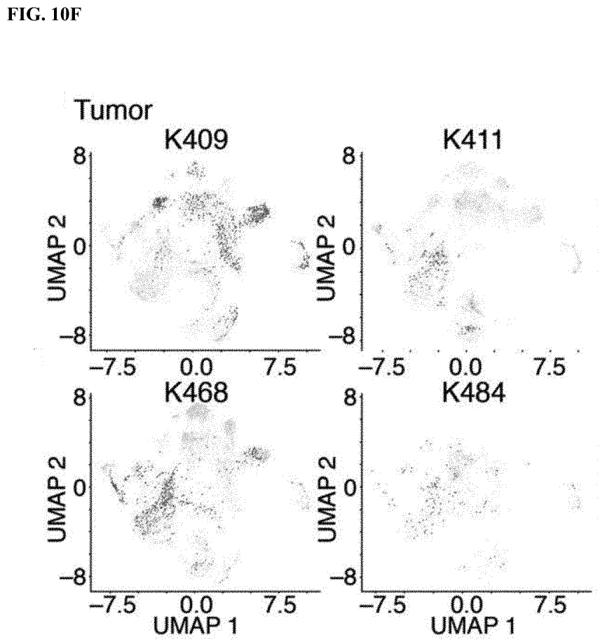

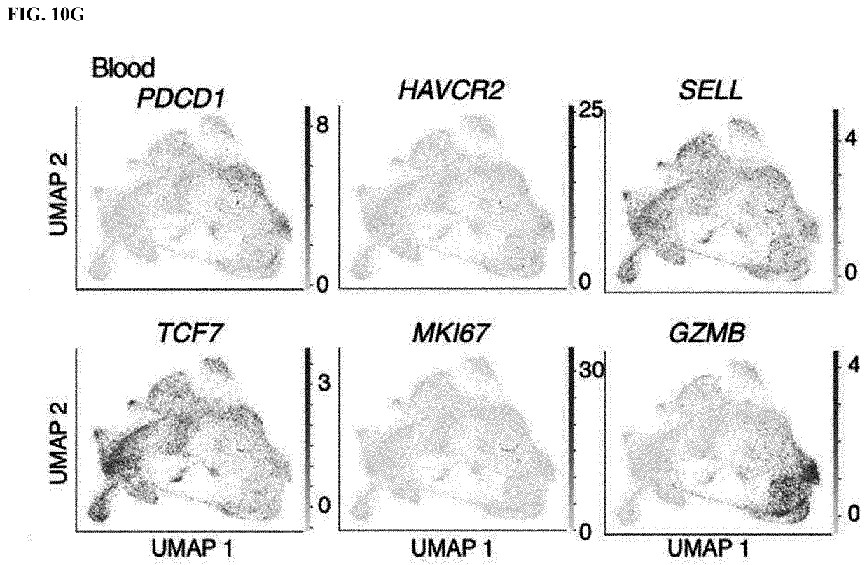

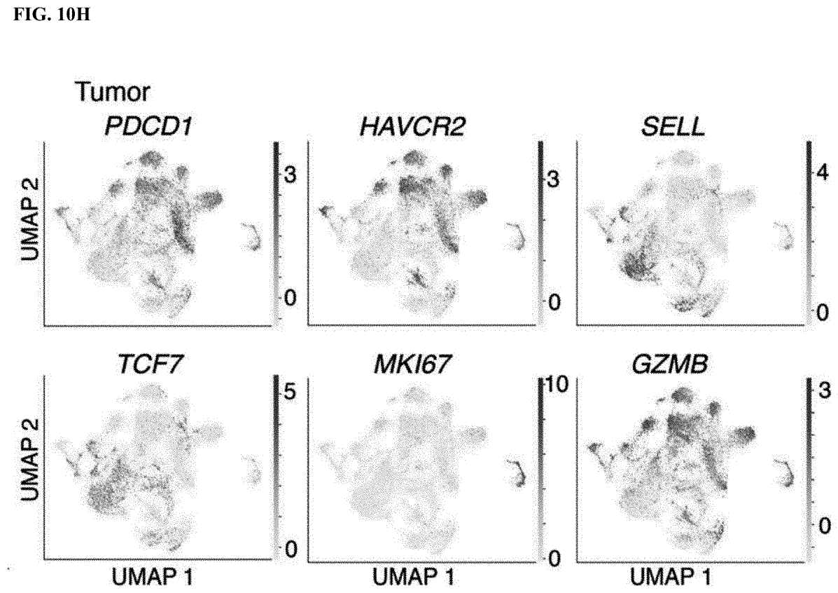

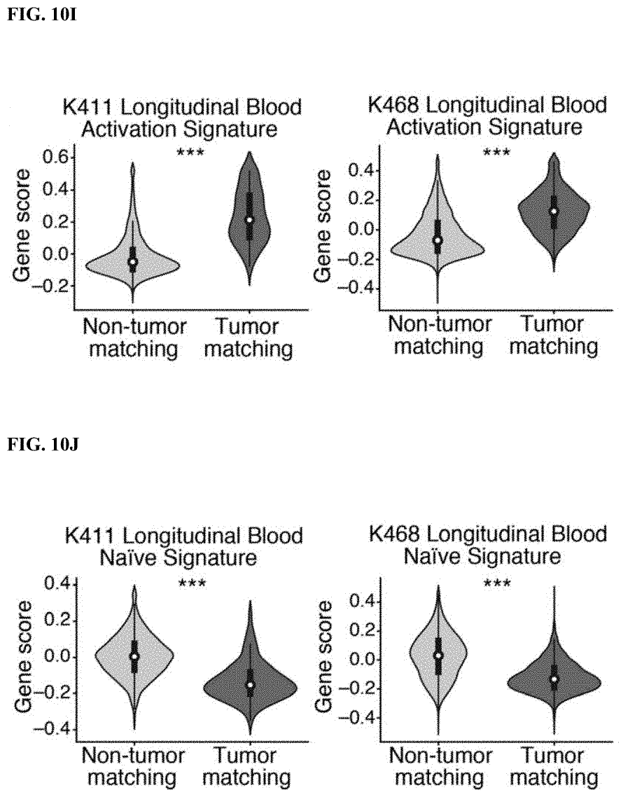

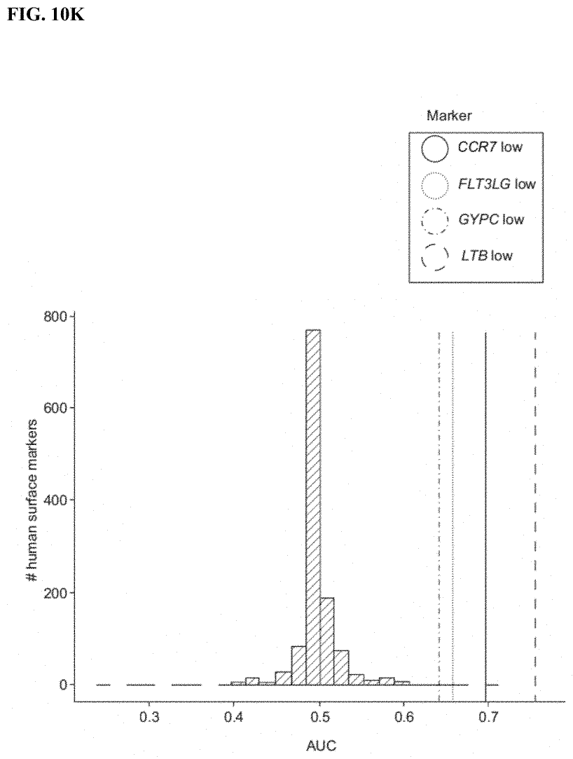

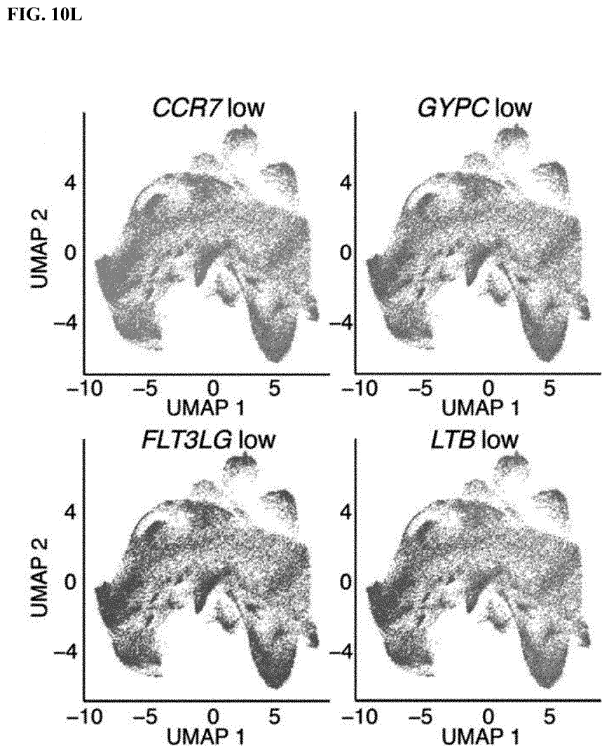

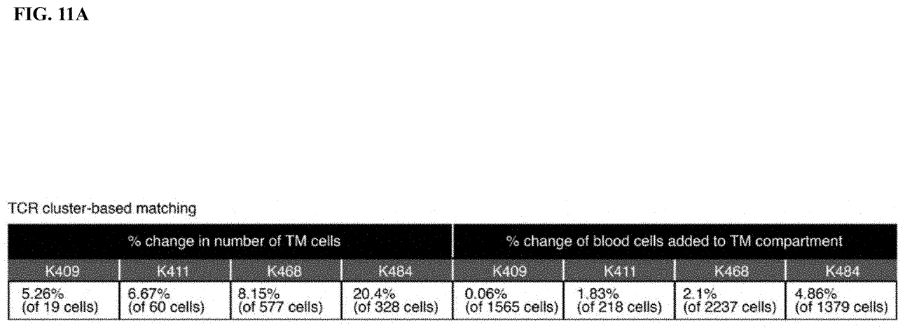

We next applied this approach to cancer patients, using single-cell RNA and TCR sequencing from four checkpoint treatment-naïve advanced melanoma patients (FIGS. 10A-10H, Table S7). Here, “tumor” refers to tissue resections obtained from the primary tumor site or metastases (FIG. 10B, Table S7). CD8⁺ T cells had transcriptional signatures in blood consistent with naïve-like, central memory-like, effector-like, and effector memory-like cells, and signatures in tumor consistent with diverse exhausted subpopulations, effector-like, resident memory-like, naïve-like and/or central-memory like, and cycling populations (FIGS. 4A-4B, Table S8), consistent with previous reports (Guo et al., 2018; Sade-Feldman et al., 2018; Siddiqui et al., 2019; Tirosh et al., 2016; van der Leun et al., 2020; Yost et al., 2019).

Next, we detected TM cells in the blood using the TCR sequence as a molecular barcode (FIGS. 4C, 10B). Despite heterogeneity across patients (FIGS. 10E-10F), the majority of TM cells in each patient were present in “non-naïve” clusters (e.g. not clusters 0, 1, or 6) (FIGS. 4A, 4C). The percentages of TM cells in these “non-naïve” clusters were 94.7% (K409), 88.3% (K411), 83.5% (K468), and 87.7% (K484). TM cells mostly belonged to clusters associated with an effector and/or effector-memory-like phenotype (clusters 2, 3, 4, and 5) (FIGS. 4A, 4C). Consistent with this, TM cells in the blood of all patients expressed significantly higher levels of an activation signature compared to non-TM cells (FIG. 4D), and non-TM cells expressed significantly higher levels of the naïve signature (FIG. 4E). To interrogate how the level of exhaustion compared between clones in blood and clones in tumor, we evaluated an exhaustion signature on a clone by clone basis between these two tissues. In patient K409, there was no significant difference in the exhaustion score between clones in blood and tumor (FIG. 4F, K409 p=0.2). However, in the other three patients analyzed, the exhaustion signature was significantly elevated on matching clones in tumor relative to blood (FIG. 4F, K411 p=4×10⁻⁵, K468 p=7.1×10⁻¹⁹, K484 p=1.9×10⁻⁷). These data are consistent with our results in mice, supporting the idea that TM cells in the blood may be less dysfunctional than their corresponding counterparts in tumor.

Tumor-matching CD8⁺ T cells can be tracked longitudinally in patient blood and show a temporal increase in exhaustion despite anti-PD-1 treatment

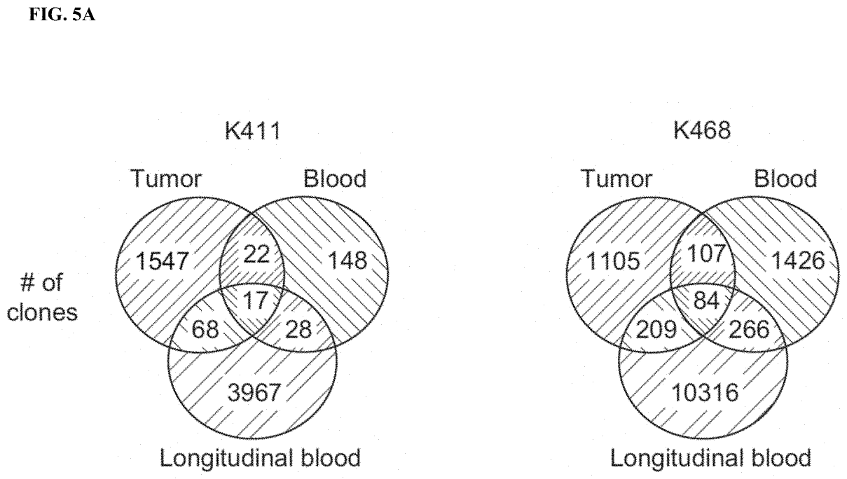

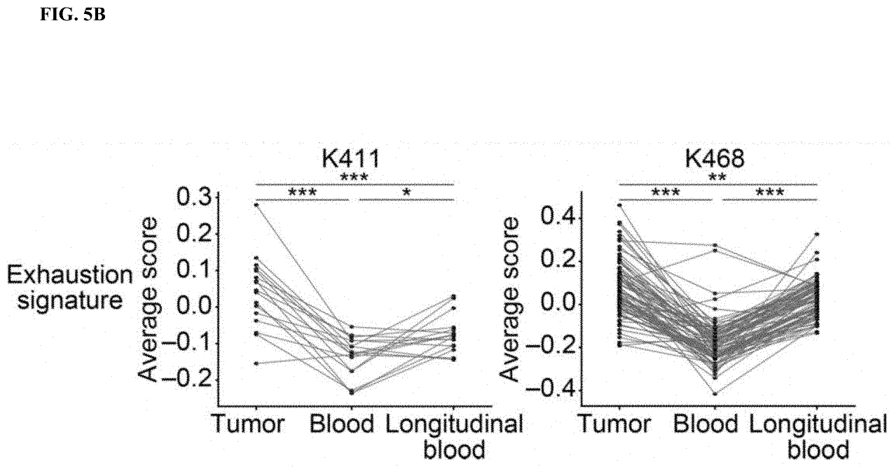

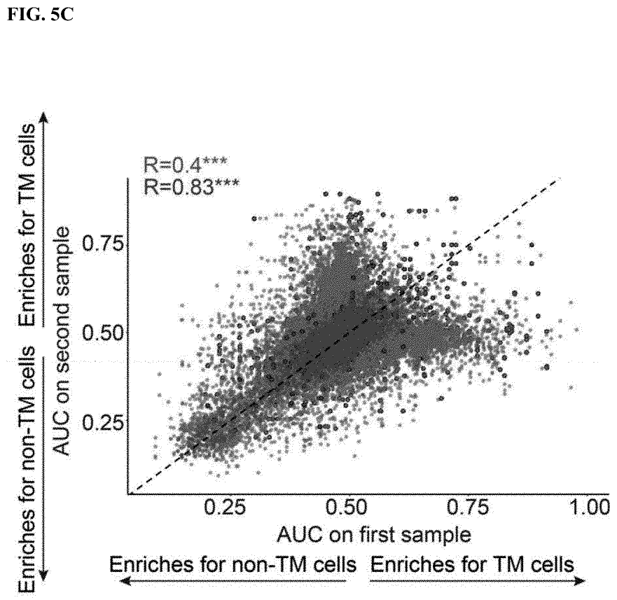

Follow up blood samples were obtained from two patients that failed to respond to checkpoint blockade, K411 and K468 (FIG. 10A). We detected overlapping TCRs between the two blood samples and the tumor sample in each patient, despite one of the samples being collected almost a year and half after the initial sample (FIGS. 5A, 10A). TM cells detected in the longitudinal samples showed increased activation compared to non-TM cells (FIGS. 10I-10J), similar to the trend in the initial sample (FIGS. 4D-4E). Notably, the exhaustion signature was higher in the longitudinal samples than the initial blood samples, but lower than the tumor (FIG. 5B). These data indicate that TM cells in the blood can become more exhausted over time despite anti-PD-1 treatment, but ultimately the highest levels of exhaustion were in the tumor.

We next quantified the extent to which the transcripts enriched in the TM component relative to the non-TM component correlate across patient samples. The extent of similarity across samples was greater for within-patient comparisons than between-patient comparisons (FIG. 5C, Table S9). Despite the acquired differences in the T cell exhaustion signature of clones following therapeutic intervention (FIG. 5B), the general transcriptional landscape of the TM component relative to the non-TM component remained highly consistent within the two patients assessed in this study (FIG. 5C, R=0.83, p-value<2.2×10⁻¹⁶).

Analysis of between-patient variability revealed a significant correlation (FIG. 5C, R=0.4, p-value<2.2×10⁻¹⁶) in the extent to which individual gene transcripts are specific to the TM component or the non-TM component. This consistency suggested there may be useful transcripts for isolating the TM component from blood. We, therefore, restricted our correlation analysis to cell surface markers (Chihara et al., 2018) since their transcripts would have practical uses (e.g. sorting for sequencing, functional assays, or adoptive cell transfer therapy), and correlations in the TM component remained (R=0.31, p-value<2.2×10⁻¹⁶). This result indicates that surface-expressed biomarkers can be defined for the TM population that are robust to varying tumor burdens and therapeutic conditions.

Cell surface marker combinations can be used to detect the TM component from patient blood

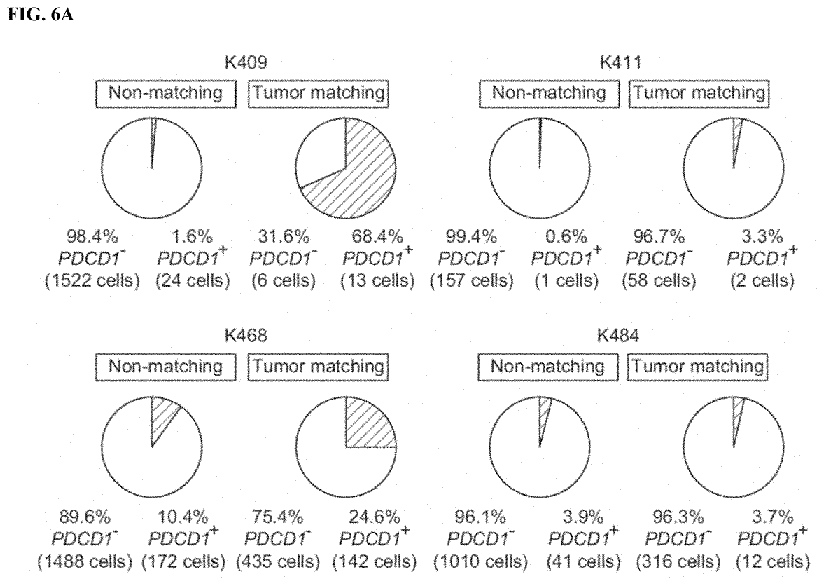

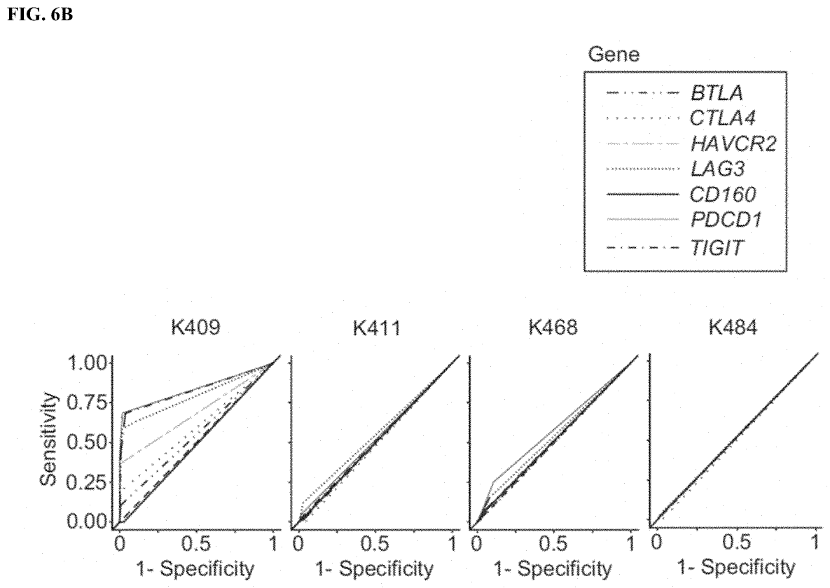

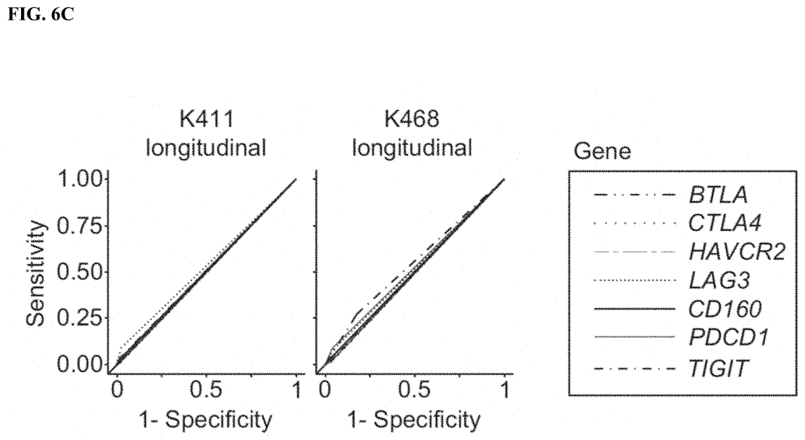

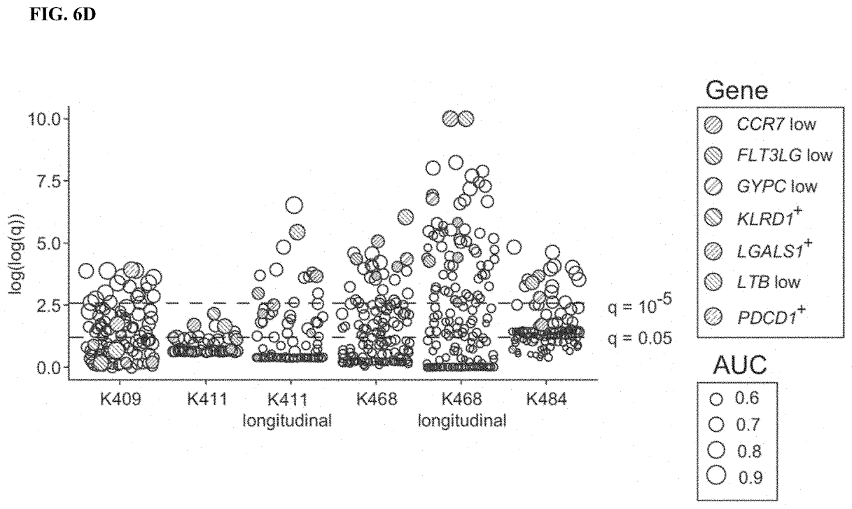

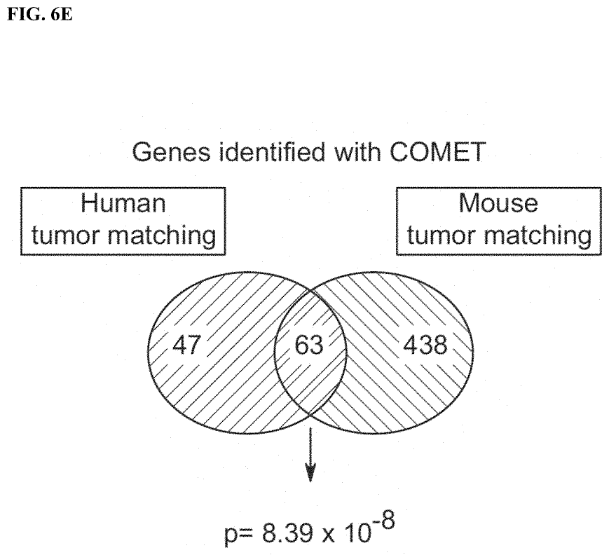

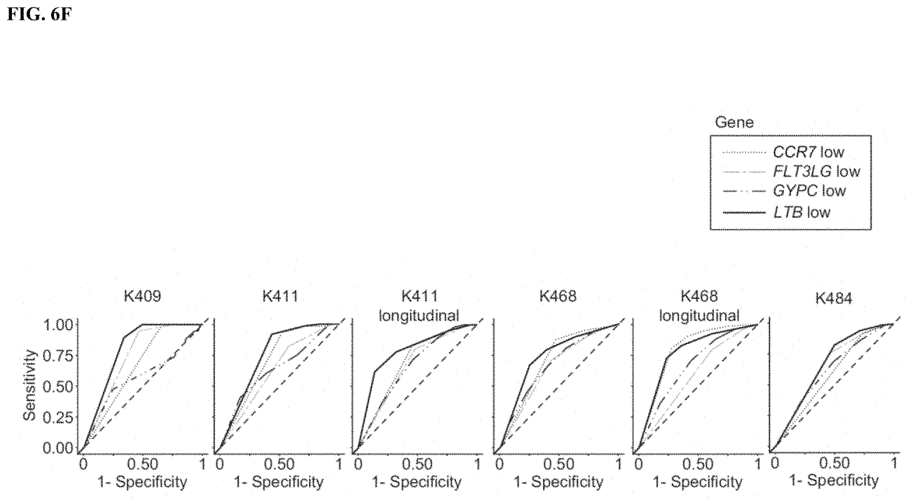

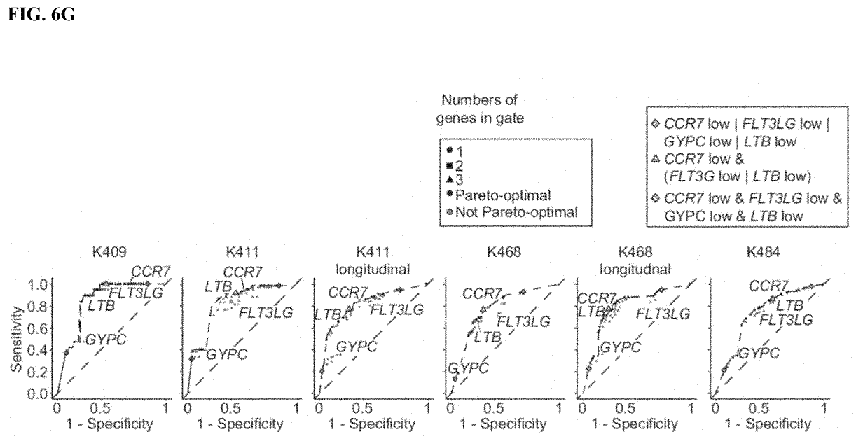

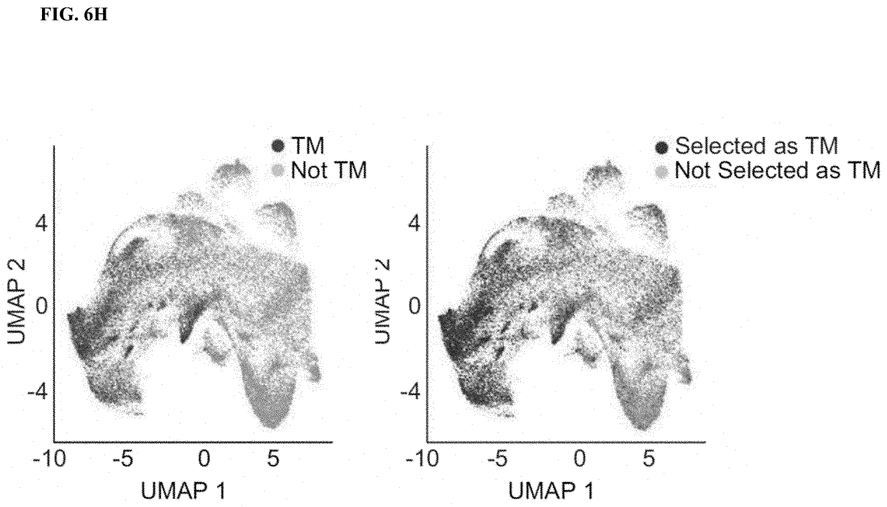

We next asked if cell surface markers could enrich TM cells. We first examined use of inhibitory receptors. With the exception of patient K409, PDCD1 RNA was detected on a minority of the TM cells (FIG. 6A). Moreover, at the transcript level, PDCD1 and a number of other inhibitory receptors had poor performance as predictive markers (FIGS. 6B-6C, Table S9). Our finding that the AUC values for the inhibitory receptors were hardly above chance for most patients indicated that this class of markers could not reliably enrich TM cells in blood. An independent study also found PDCD1 to be a poor marker for cells in patient blood with TCRs matching to those in paired melanoma samples (Lucca et al., submitted).

To determine better surface markers for TM cells in humans, we again used COMET to identify transcripts that significantly enriched for TM cells (FIG. 6D, Table S10). We observed a significant overlap between markers for the TM compartment in patient samples and the markers in mice (FIG. 6E, Table S11), indicating that some markers of TM cells are conserved across species and cancer types. We identified 15 near-consensus surface markers that had q<0.05 in at least four of the six patient samples (Table S12). Of the 15 near-consensus genes, many were considered low or absent on TM cells (e.g., those for which positive expression denotes that a cell is more likely to be non-TM, see Methods). The top four ranking markers based on AUC were a lack of LTB, CCR7, GYPC, and FLT3LG on TM cells (referred to as LTB^{low}, CCR7^{low}, GYPC^{low} and FLT3LG^{low}). Low expression of these markers is consistent with the “non-naïve” and/or “effector or effector memory-like” transcriptional state of TM cells (FIGS. 4A, 4C). These markers showed consensus despite differing tumor burdens and therapeutic states, showing robust AUC performance (CCR7^{low}: 0.742; FLT3LG^{low}: 0.619; GYPC^{low}: 0.647; LTB^{low}: 0.772; empirical p<0.0001 for each) (FIGS. 6D, 6F, 10K, Table S13). However, these markers featured differing strengths in sensitivity in specificity; CCR7^{low} 0.827 sensitivity and 0.621 specificity, GYPC^{low} 0.340 sensitivity and 0.819 specificity, FLT3LG^{low} 0.780 sensitivity and 0.447 specificity, and LTB^{low} 0.718 sensitivity and 0.768 specificity (empirical p<0.0001 for each, FIGS. 6F, 10L, Table S13). Though these top four markers are negation markers (e.g. low/negative expression on TM cells), we did observe some positive markers for TM cells lower on the list, including KLRD1 and LGALS1 (FIG. 6D, Table S12), which came up in a companion study co-submitted with this work (Lucca et al., submitted).

To increase performance of surface markers to isolate TM cells from blood, we next explored the use of combinations. In all samples, marker combinations of two or more genes significantly improved performance on sensitivity and/or specificity over single markers (FIGS. 6G-6H, Table S14). The best-performing gate with even balance between sensitivity (0.745) and specificity (0.745) was [(CCR7^{low} and FLT3LG^{low} and GYPC^{low}) or LTB^{low}] (meaning that a cell either has low LTB, or low expression of any of the other three markers) (empirical p<0.0001 for each) (FIGS. 6G-6H, Table S13). Collectively, these data highlight the utility in using combinations of markers to enrich TM cells.

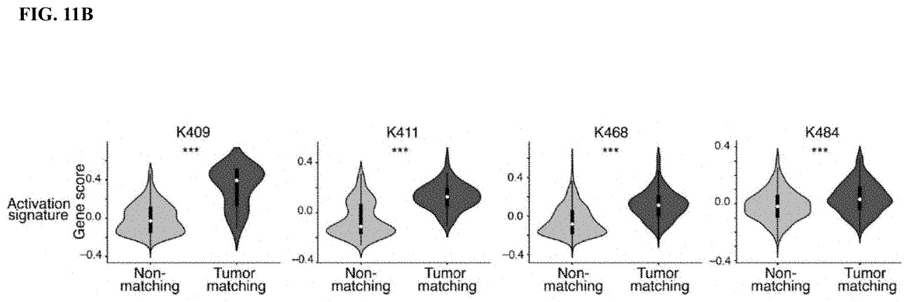

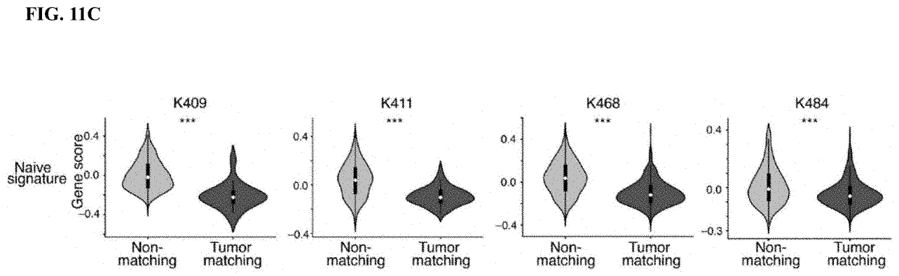

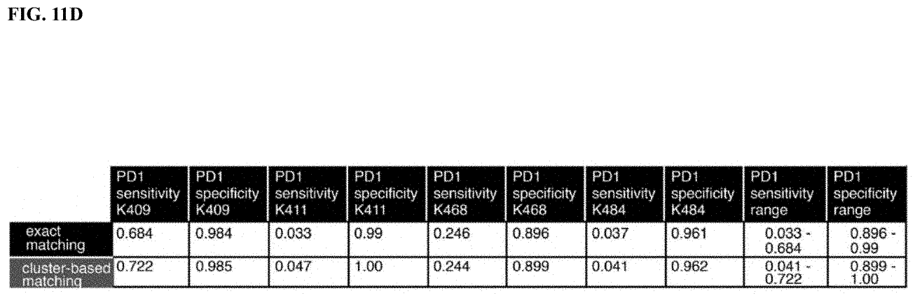

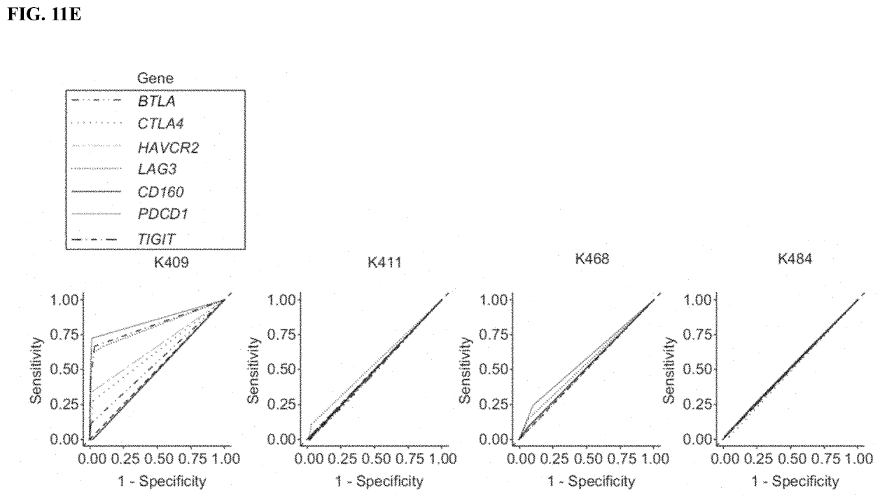

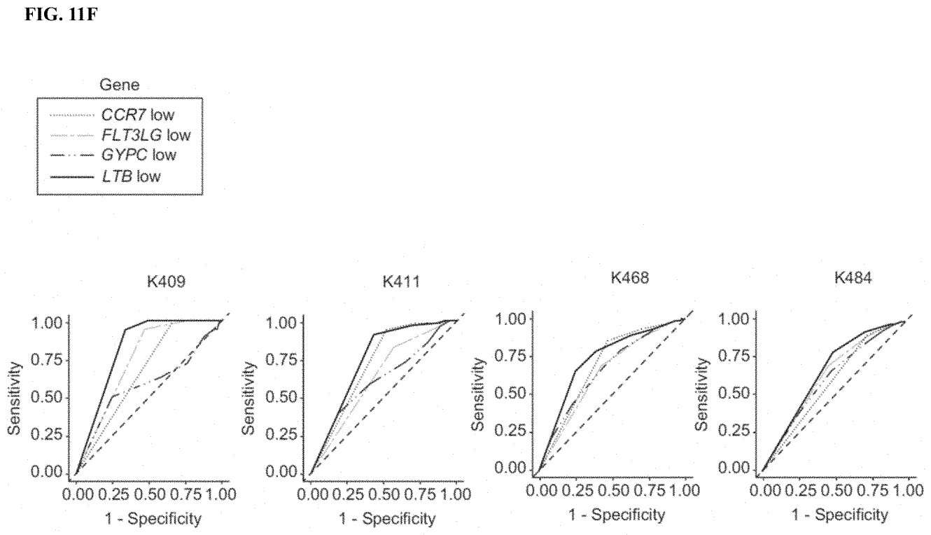

Lastly, some TM cells may have been missed since an exact sequence match for both the alpha and beta chain is a highly stringent definition of a clone. To address this issue, we utilized two additional TCR clustering tools, GLIPH2 (Huang et al., 2020) and iSMART (Zhang et al., 2020), which increased the number of TM cells recovered (5.26%-12.9%) (FIG. 11A). However, TM cells were still enriched in an activation signature (FIG. 11B), and non-TM cells were still enriched in a naïve signature (FIG. 11C). Additionally, the sensitivity of PDCD1 and the other inhibitory receptors remained insufficient overall (FIGS. 11D-11E, Table S15). In contrast, the AUC performance of CCR7^{low}, FLT3LG^{low}, GYPC^{low}, and LTB^{low} remained high (FIG. 11F, Table S15). The results show that marker panels can be built to monitor patients' responses to immunotherapy in real time.

Discussion

There is tremendous interest in monitoring anti-tumor immune responses. The blood is a conduit of immune cell trafficking, making it a window into these host immune responses. However, comprehensive profiling of tumor antigen-specific T cells in the blood has been challenging. Use of the TCR as a molecular barcode to track TM cells provides an effective way to enrich tumor-relevant cells. This approach is less biased than alternatives like PD-1 expression, and captures a larger breadth of the anti-tumor response than individual peptide/MHC tetramers, enabling more focused and statistically powered analyses.

There are technical and biological considerations with this method. First, paired blood and tumor samples are required to identify TM cells. Second, sampling depth in the tumor may impact the proportion of the TM repertoire detected. However, the TM cells detected here showed consistent transcriptional states and markers for their isolation despite variability in the depth of coverage across patients. Third, use of negation markers can be challenging in single cell data, since these data sets contain a large number of zero values and it is has been debated whether counts of zero are due to true biology or technical artifacts. It is generally accepted that genes receiving zero counts are either not expressed or expressed to a low extent within a cell (Choi et al., 2020), and recent work has concluded that the zero measurements in count data reflect true biology (Choi et al., 2020; Hafemeister and Satija, 2019; Svensson, 2020; Townes et al., 2019). We therefore conclude, from the highly significant proportion of zeros and low count measurements in TM cells compared to non-TM cells as detected by COMET, that TM cells are lower for GYPC, CCR7, LTB, and FLT3LG than non-TM cells. Fourth, bystander T cells specific for pathogens have been identified in mouse and human tumors (Mognol et al., 2017; Simoni et al., 2018).

The majority of TM cells in the blood of our advanced melanoma patients displayed an effector and/or effector-memory-like phenotype. This was counter to our predictions, where we expected a more exhausted-like profile. While patient K409 showed similar enrichments for an exhaustion signature between matching clones in blood and tumor, the other three patients showed elevated exhaustion scores in the tumor. This finding highlights the importance of using the TCR rather than surrogate markers such as PD-1, which make an assumption about expected differentiation states of relevant cells. As a class, inhibitory receptor transcripts performed poorly at distinguishing TM cells in the blood with the exception of patient K409, indicating that there may be better cell surface markers for identifying tumor-relevant cells in blood.

Three markers validated here in mice for identifying TM cells were NKG2D, CD39, and CX3CR1. When comparing effector, memory, and exhausted populations, Klrk1 shows the highest expression in memory CD8⁺ T cells (assessed from GSE41867, (Doering et al., 2012)), and NKG2D is important for optimal memory formation (Andre et al., 2012; Ferrari de Andrade et al., 2018; Prajapati et al., 2018; Wensveen et al., 2013; Zloza et al., 2012). CD39 is associated with exhaustion (Gupta et al., 2015). CX3CR1 correlates with effector CD8⁺ T cell differentiation, with the highest levels on the most effector-like cells (Gerlach et al., 2016). How NKG2D, CD39, CX3CR1, and other candidate markers impact the function of TM cells remains to be determined. Some of the markers identified may be specific to this particular tumor type or its location in the skin, and may differ with tumor type or location. However, a number were associated with a general program of trafficking to inflamed tissues and not skin specific, including Ccr2, Ccr5, Cx3cr1, Itga4, Itgb1, and Itgb2 (Liu et al., 2006; Masopust et al., 2010). The significant overlap between TM markers in the mouse MC38 model and melanoma patients (FIG. 6E and Table S11) indicates that there can be similarities that span tumor type and species.

In mice, the TM population in the blood was fairly homogenous. However, blood-matching clones in tumors showed significant transcriptional diversity. These data indicate that TM cells have a high degree of plasticity upon entering the tumor, and the tumor microenvironment influences the development of diverse functional states. On a clonal basis, TM cells in the blood were less exhausted than their blood-matching counterparts in tumor in both mice and patients with the exception of K409. In the two longitudinal patient samples, the clones detected in the second blood sample were more dysfunctional than the first, consistent with the notion that exhaustion continues to develop over time (Wherry and Kurachi, 2015). However, clones in blood appeared less exhausted than clones in tumor, indicating that blood may be a reservoir of less dysfunctional cells.

In summary, we identified CD8⁺ T cells in blood that had matching TCRs with CD8⁺ TILs in both mouse and human tumors. TM cells in blood were generally less dysfunctional than matching clones in tumor. Additionally, we provide evidence for an exciting and tractable innovation: the use of combinatorial marker panels to isolate TM cells in blood. These panels were consistent over time, across patients, and robust to sampling variation. Follow up studies interrogating how immunotherapies such as PD-1 blockade impact TM cells will be highly relevant to determining predictors of response versus resistance. Our algorithmic approach to generate marker panels to identify TM cells coupled with future longitudinal studies could assist with creation of diagnostics, potentially allowing monitoring of the anti-tumor immune response in real time without the need for single-cell sequencing.

Materials and Methods

Experimental Model and Subject Details

Mice and Cell Lines

Wild type (WT) female C57BL/6 mice were purchased from the Jackson Laboratory (stock number 000664). Tumor cells were implanted into mice at 8-10 weeks of age. Mice were maintained at Harvard Medical School in specific pathogen-free facilities under standard housing, husbandry, and diet conditions in accordance with Institutional Animal Care and Use Committee (IACUC) and NIH guidelines. All experimental procedures performed were approved by the IACUC at Harvard Medical School.

For tumor studies, MC38 colon adenocarcinoma cells (a gift from Dario Vignali, University of Pittsburgh School of Medicine) were used. MC38 cells were grown in DMEM supplemented with 10% FBS, 100 U penicillin, and 100 μg streptomycin in a 37° C. incubator with 5% CO₂. Cells were harvested at passage 2-3 after thaw, and 2.5×10⁵ tumor cells were injected subcutaneously into the flank of mice anesthetized with 2.5% 2,2,2-Tribromoethanol (Avertin). Tumors were measured every 2-3 days using calipers, and mice were sacrificed when tumors reached 2 cm³ volume, ulceration, or a body condition of >2 in accordance with IACUC guidelines. Tumor volume was determined using the formula for the volume of an ellipsoid, ½×D×d², where “D” is the major axis of the tumor and “d” is the minor axis. Tumors were harvested from mice at days 18-23 after implantation for single cell RNA sequencing experiments and flow validation experiments as indicated in the Figure Legends.

Clinical Samples

Studies of patients with melanoma were approved by the UCSF Committee on Human Research (CC138510) and by the Institutional Review Board of UCSF under protocol 13-12246. All patients provided written, informed consent prior to biopsy and/or blood collection. Patient sample details including location of biopsy, treatments following initial blood/tumor sampling, gender, age, and timing of longitudinal blood collection can be found in FIGS. 10A-10B, and Table S7.

Method Details

Lymphocyte Isolation from Mouse Tissues

Peripheral blood was collected from mice using the retroorbital bleeding route, and blood was collected into 4% sodium citrate (Sigma) to prevent clotting. RPMI+10% FBS was added to dilute out the anti-coagulant, and then white blood cells were separated from red blood cells using centrifugation through histopaque-1083 (Sigma). The white blood cell layer at the interface between the histopaque and remaining media was subsequently subjected to staining for flow cytometry analysis or sorting for single cell RNA sequencing.

Tumors were dissected and mechanically disaggregated. For flow cytometry validations, a GentleMACS (Miltenyi) was used for disaggregation, whereas for single cell RNA sequencing vertical scissors were used to mince the tumors instead of the GentleMACS. The dissociated tissue was digested with Collagenase Type I (400 U/ml; Worthington Biochemical) for 20-30 minutes at 37° C. Samples were then passed through a 70 μm filter, and lymphocytes were enriched using centrifugation through a Percoll gradient (40% and 70%). The enriched lymphocyte layer at the 40%/70% interface was subsequently stained for flow cytometry or sorted for single cell RNA sequencing.

Flow Cytometry and Sorting of Mouse Samples

Single cell suspensions were generated as described above. Suspensions were labeled with LIVE/DEAD Fixable Near-IR Cell Stain in PBS (Thermo Fisher Scientific) to exclude dead cells from downstream analyses. Cells were pre-incubated with TruStain Fc Receptor Block (anti-mouse CD16/CD32, clone 93, BioLegend), then labeled with extracellular antibodies including: CD3 (clone 145-2C11) and CD8a (clone 53-6.7) (from BD); CD11a (clone M17/4) (from Thermo Fisher Scientific); CCR2 and NKG2I (from R&D Systems); Lag3 (clone C9B7W) (from Bio-Rad); and CD45.2 (clone 104), PD-1 (clone RMPI-30), CX3CR1 (clone SA011F11), CD62L (MEL-14), CD44 (IM7), CCR5 (clone HM-CCR5), CXCR6 (clone SA051D1), CD49D (clone R1-2), CD18 (clone M18/2), CD29 (clone HMβ 1-1), CD48 (clone HM48-1), CD94 (clone 18d3), NKG2D (clone CX5 or C7), CD39 (clone Duha59), NKG2A (clone 16A11), NK1.1 (clone PK136), Tim-3 (clone RMT3-23), CD160 (clone 7H1), Slamf7 (clone 4G2), TIGIT (clone IG9), and NRP1 (clone 3E12) (from BioLegend). Flow cytometry labeling (without inclusion of Feature Barcoding antibodies from BioLegend) was performed in PBS supplemented with 2% FBS. For CITE-seq validation experiments, cells were labeled with TotalSeqC antibodies against CD39 (TotalSeq C0834, clone Duha59) and CX3CR1 (TotalSeq C0563, clone SA011F11) as directly conjugated antibodies, and NKG2D as a biotin/streptavidin reaction (NKG2D-biotin clone C7 paired with TotalSeq C0971-Steptavidin) (from BioLegend). Labeling with Feature Barcoding antibodies was performed in PBS supplemented with 2% BSA and 0.01% Tween. Samples were acquired on a FACSymphony (BD Biosciences) and analyzed with Flow Jo software (BD Biosciences). Flow cytometry-based sorting for single cell RNA seq was performed using a FACSAria (BD Biosciences). Because we expected TM cells in the blood to be rare, we sorted for CD44⁺ CD8⁺ T cells to enrich for antigen-experienced populations in the blood (full sorting strategy=live, CD45.2⁺, CD3⁺, CD8α⁺, CD44^{mid-high}.) Although all CD8⁺ T cells sorted from blood expressed some level of CD44, cells from Mouse 1 (or M1, experiment 1) were sorted on CD44^{high}, while cells from Mouse 2 and Mouse 3 (or M2 and M3, experiment 2) included both CD44^{mid} and CD44^{high} cells. Tumor samples were sorted based on live, CD45.2⁺, CD3⁺, CD8α⁺.

Single Cell RNA Sequencing of Mouse Samples

Gene expression and TCR libraries for mouse samples were generated using the Chromium Single Cell 5′ Library and V(D)J Reagent Kit (10× Genomics) according to the manufacturer's recommendations. For samples requiring Feature Barcoding libraries to detect TotalSeqC antibodies (from BioLegend), the Chromium Single Cell 5′ Feature Barcode Library Kit (10× Genomics) was used according to the manufacturer's recommendations. Following sorting as described above, approximately 10,000 cells per sample were loaded into each channel of the Chromium Chip, and recommendations were followed assuming targeted cell recovery of 2,001-6,000 cells. Libraries were sequenced on a NextSeq sequencer (Illumina) by the Dana-Farber Cancer Institute Sequencing Core. Gene expression libraries and Feature Barcoding libraries were sequenced using the 26×8×91 bp parameters recommended by 10× Genomics. TCR libraries were sequenced using the 150×8×150 bp parameters recommended by 10× Genomics. Based on approximate cell numbers expected, we sequenced a minimum of 20,000 reads per cell for gene expression libraries and 5,000 reads per cell for TCR and Feature Barcoding libraries.

Lymphocyte Isolation from Human Tissue Samples

Human melanoma tumor samples were mechanically dissociated and enzymatically digested overnight for 12-14 hours. Following fine mincing with scissors, samples were digested in RPMI media (Gibco) containing 250 U/mL Type IV collagenase (4188; Worthington Biochemical Corp.), 20 μg/mL DNAse (SDN25-1G; Sigma-Aldrich), 10% FBS (Alphabioregen), 1% HEPES (Gibco), 1% penicillin/streptomycin (Gibco), and 2 mM glutamine (GLUTAmax, Gibco) at 37° C. in a tissue culture incubator with 5% CO₂. Following overnight incubation, digestion was quenched with excess media, and samples were transferred to 50 mL conical tubes and briefly shaken, and then were filtered through a 100 μm sieve. Samples were pelleted and washed in media before downstream applications.

Lymphocyte Isolation from Human Blood Samples

Blood from patients with melanoma was collected in heparinized or EDTA tubes and diluted with an equal volume of PBS before being layered over a Ficoll Paque PLUS gradient (GE Healthcare) in 50 mL conical tubes that were centrifuged at room temperature for 15 minutes at 932 g. Cells were isolated from the Ficoll/PBS interface and washed at least twice in PBS/2% FBS before downstream applications. For the two patients with longitudinal blood samples processed (K411 and K468), both patients still had tumor at the time of longitudinal blood collection.

Flow Cytometry and Sorting of Human Samples

Melanoma tumors (primary tumors or metastases as indicated in FIG. 10B and Table S7) or blood were stained in PBS with Tonbo Ghost Dye Violet 510, anti-CD45 (Clone H130), anti-CD3 (Clone SK7), anti-CD4 (Clone SK3), anti-CD8 (clone SK1). Some samples were additionally stained with anti-PD-1 (Clone EH12.2H7), anti-CD25 (Clone M-A251), anti-CD27 (Clone LG.7F9), anti-CD127 (Clone HIL-7R-M21). CD8⁺ T cells were sort-purified as singlet, live, CD45⁺, CD3⁺, CD4⁻, CD8⁺ events on an Aria 2 or Aria 3u (BD) in the UCSF Parnassus Flow Cytometry Core. In some cases the total CD3⁺ T cell population was sort-purified as singlet, live, CD45⁺, CD3⁺ events, and CD8⁺ T cells were identified bioinformatically. Cells were counted post-sort on a hematocytometer and resuspended to target ˜1000 cells/μL in media with 10% FBS for single-cell RNA Sequencing.

Single Cell RNA Sequencing of Human Samples

Following sorting, cells were prepared for single-cell RNA Sequencing using the 10× Chromium Platform (10× Genomics) by the Institute for Human Genetics at UCSF. Cells were processed following the recommended protocol with the Chromium Single Cell 5′ Library Construction kit and Chromium Single Cell V(D)J Enrichment Kit (Human T Cell) (Single Cell 5′ PE Chemistry). Libraries were run on a HiSeq 4000. FASTQ files were generated and analyzed with Cell Ranger (v3.0.2) by the UCSF 10× Genomics Core using the GRCh38 human reference genome for alignment.

Demultiplexing and Read Processing

Raw reads were processed using Cell Ranger v3.0.2 to generate raw counts matrices of gene expression and csv files corresponding to TCR clonality. The GRCh38 human reference genome was used for alignment of human samples, and the mm10 mouse reference genome was used for alignment of mouse samples. Aether version 1.0 (Luber et al., 2018) was used to process certain resource heavy jobs on compute instances rented from Amazon Web Services.

Computational Processing of Gene Expression Data

All analyses were conducted using R version 3.6.1 and Seurat version 3 with additional utilization of the dplyr, data.table, ggplot2, cowplot, viridis, gridExtra, RColorBrewer, ggpubr, ggrepel, gtools, DescTools, doParallel, doSNOW, and tibble packages. Seurat objects were created with the min.cells parameter set to 3 and the min.features parameter set to 400. Filtering cells based on expression of housekeeping genes was conducted using the human and mouse (where appropriate) gene lists maintained by the Seurat developers (available on the Satija lab website), with cells passing the filtering criteria if they had expression greater than 0 for more than half of the genes in the list. Subsequently, the MitoCarta database from the Broad institute was utilized to filter out cells based on expression of mitochondrial genes (Calvo et al., 2016). Cells were filtered out if they expressed more than 500 of the 1158 mitochondrial genes in human, or if the number of mitochondrial genes expressed was higher than 2 standard deviations from the mean in mouse.

Data were normalized using the default Seurat function (generating log-transformed transcripts-per-10K read measurements) followed by scaling, and variable genes were found using “ExpMean” for the mean.function parameter and “LogVMR” for the dispersion.function parameter. The RunPCA function was run utilizing 50 principal components and then the FindNeighbors function was run using 30 dimensions. Subsequently, the FindClusters function was run with a resolution aiming to generate 5-7 biologically meaningful clusters per sample. To filter for CD8⁺ T cells in humans, clusters were kept if (1) the proportion of cells in the cluster with at least two genes out of CD3E, CD3D, or CD3G being expressed was greater than 30% and either (2) CD8B was expressed in more than 30% of cells in the cluster, CD8A was expressed in more than 30% of cells in the cluster, FOXP3 was expressed in less than 5% of cells in the cluster, and CD4 was expressed in less than 5% of cells in the cluster or (3) MKI67 was expressed in greater than 70% of cells in the cluster and either CD8A or CD8B was expressed in greater than 20% of the cells in the cluster. This last criterion was to account for proliferating clusters. In mice, which had less contamination from non-CD8⁺ T cells due to prior sorting, clusters were kept if more than 30% of cells in the cluster expressed any of Cd3e, Cd3d, or Cd3g and: if more than 30% of cells in the cluster expressed Cd3e and Cd8a while having less than 5% of the cells express Foxp3. When applicable, samples were integrated using the SCTransform method (Hafemeister and Satija, 2019).

The samples from the first three mice with MC38 tumors were integrated to generate an “integrated blood” sample and an “integrated MC38 tumor” sample as a discovery cohort. These three mice were generated between two independent experiments (M1=experiment 1, M2 and M3=experiment 2). Mouse 4 and Mouse 5 were generated as a separate validation cohort which included CITE-seq. For patient samples, the majority of analyses were performed on each patient individually, not the integrated sample. Integration was performed for clustering and UMAP visualization purposes, and included only the initial four pre-treatment samples with the exception of FIG. 6H, which included all six samples (the initial four pre-treatment samples and the two longitudinal samples). For patient K409, tissue from both the primary tumor site and an involved LN was processed for scRNA seq. For this patient, the data for primary tumor and the involved LN were pooled, and cells in the blood were considered TM if they had a TCR sequence matching to either tumor resection.

Upon obtaining transcriptional clusters in the integrated datasets, up regulated genes associated with each cluster were determined via the Wilcox Rank Sum test implemented in the FindAllMarkers function in Seurat. Cells were classified as positive for the PD-1 transcript (Pdcd1 in mice, PDCD1 in humans) if they had any number of reads above zero. To classify mouse cells as positive for the Klrk1 (encoding NKG2D), Entpd1 (encoding CD39), and/or Cx3cr1 (encoding CX3CR1), a more stringent cut off was used for a cell to qualify as positive, determined by COMET (Delaney et al., 2019).

For enrichment analysis tests (FIG. 1E), all genes were ranked by their p-value and by their fold change, and then the two ranking values were aggregated to create a single ranking by taking the mean of the p-value and fold-change rankings. We then searched for significant associations with gene signatures by using the ranked list in the PreRanked analysis of GSEA (Subramanian et al., 2005). Default settings were used except: permutations was set to 100, the enrichment statistic set to ‘classic’, and the max size set to 2500. The signature sets used were all GO terms, Kegg and Reactome pathways, and immune signatures from MSigDB (groups c2, c5, and c7). Gene signatures derived from the literature were also analyzed as cited in the figures and manuscript.

In order to perform the clonal-corrected DE gene analysis comparing TM cells in the blood to blood-matching cells in the tumor (Table S6), the non-normalized integrated mouse blood object was subsetted to keep only TM cells, which were then collapsed into their clones such that for each gene, the counts for all the cells in a clone were summed together. This was done for the integrated tumor as well, but with blood-matching cells. The tumor and blood-derived datasets were then merged to a single object, and the edgeR package was used to call differential expression. Genes were considered if they expressed at least 1 count per million, and then counts were normalized using the trimmed mean of M-values. Taking into account the paired nature of matching clones in blood and tumor, genes were fit to a generalized linear model using the ‘glmFit’ function, and likelihood ratio tests were conducted to detect DE genes between blood and tumor with the ‘glmLRT’ function.

Single-Cell TCR and Clonal Analysis

Cells for which at least one alpha and one beta chain were annotated in the TCR data were determined as “tumor/blood-matching” or “tumor/blood-non-matching” based on whether there was a cell in the paired tissue data that had the exact same alpha and beta chain composition as the given cell. Only cells that had at least one alpha chain and one beta chain annotated were included in all of the analyses comparing “matching” to “non-matching” cells. Two cells were assigned to be in the same clone if they had the both the exact same alpha and beta chains assigned based on the amino acid sequence. If cells had more than one alpha and beta chain, they were considered matching if all of the alpha and beta chains detected were shared. This strict definition was used to ensure each pair of cells within the same clone had complete similarity of the TCR chains detected, and hence was with high probability derived from the same T cell clone. TCR information was also used to quantify clonal expansion. The extent of clonal expansion was determined by counting the number of cells in each clonotype.

To define tumor-matching status by clustering of TCRs, two algorithms were employed: GLIPH2 (Huang et al., 2020) and iSMART (Zhang et al., 2020). For each patient, the joint collection of blood and tumor CD8⁺ TCRs were submitted to each algorithm individually for clustering on default parameters. All resultant clusters that included at least one TCR found in the tumor sample were considered to represent reactivity to a tumor antigen, and therefore all blood CD8⁺ T cells with TCRs belonging to these clusters were considered TM cells. In general, the results of GLIPH2 and iSMART were concordant with 4,557 cell TM labels in agreement and 74 in disagreement. To buffer this analysis against variation in algorithm and parameter choices, we disregarded the 74 cells for which the two algorithms gave conflicting results (<1.6% of cells).

Functional Annotations of Seurat Clusters

Functional annotations for Seurat clusters were manually curated using a combination of up regulated genes for each cluster (Table S1 for mouse, and Table S8 for human) and visual inspection of key markers using UMAP visualization. Key markers used for aiding in annotation included: Sell, Tcf7, Lef1, Ccr7, Il7r, S1pr1, Klf2, Cxcr3, Klrg1, Cx3cr1, S1pr5, Tnf, Ifng, Il2ra, Gzmb, Prf1, Mki67, Slamf6, Pdcd1, Lag3, Tigit, Cd160, Havcr2, Ctla4, Bst2, Irf1, Irf2, Irf7, Mx1, Ccr6, Rorc, Cxcr6, Itgae, cd69, Tbx21, and Eomes. Transcriptional signatures in blood consistent with naïve, central memory, effector, and effector memory cells, and signatures in tumor consistent with diverse exhausted subsets, effector-like, resident memory-like, naïve/central-memory like, IFN-stimulated, and cycling populations, as previously reported (Best et al., 2013; Guo et al., 2018; He et al., 2016; Im et al., 2016; Kakaradov et al., 2017; Kurtulus et al., 2019; Miller et al., 2019; Milner et al., 2017; Sade-Feldman et al., 2018; Siddiqui et al., 2019; Tirosh et al., 2016; van der Leun et al., 2020; Yost et al., 2019).

Clusters that expressed high levels of Sell, Tcf7, Ccr7, Il7r, S1pr1, and Klf2 and lower levels of Klrg1, Cx3cr1, S1pr5, Tnf, Ifng, Gzmb, Prf1, Mki67, and the inhibitory receptors (e.g. Pdcd1, Havcr2, Ctla4, etc.) were considered naïve and/or central-memory like. Clusters that expressed high levels of Klrg1, Cx3cr1, S1pr5, Tnf, Ifng, Gzmb, and Prf1 and low levels of Sell, Tcf7, Lef1, Ccr7, and Il7r were considered effector and/or effector-memory like. Exhausted subsets were classified as those expressing multiple inhibitory receptors (Pdcd1, Havcr2, Lag3, Tigit, etc.), low levels of naïve and/or central memory-like markers, and generally lower levels of some effector molecules such as Klrg1 and Cx3cr1. The exhausted populations were further subdivided into progenitor-like (based on expression of Tcf7 and lower levels of Havcr2), intermediate-like (based on low levels of Tcf7 and Havcr2 and expression of other IRs including Pdcd1, Ctla4, Lag3, CD160, etc.), and terminal-like (based on high levels of multiple inhibitory receptors including Pdcd1, Havcr2, Ctla4, Lag3, Cd160, etc.). An interferon-stimulated cluster was defined based on over representation of IFN responsive genes in the up regulated gene list, including Bst2, Irf1, Irf2, Irf7, Stat1, Stat2, and Mx1. Clusters containing cells that were undergoing cell cycle were identified based on over representation of cell cycle genes (including Mki67 and several Kif, Cdk, and Cdc genes). Lastly, resident memory-like populations were identified based on expression of Itgae, Itgal, and Cxcr6.

Transcriptional Signature Analysis

We computed the extent to which gene signatures were expressed in cells by using Scanpy's ‘score_genes’ function on the centered and scaled gene count data objects (Wolf et al., 2018). Because gene signature computation is relative (following centering and scaling of the gene expression data), data of all cells compared were merged prior to the centering and scaling procedure. Violin plots were generated with the ‘seaborn’ package in Python. Signatures were derived or obtained from previously published datasets. For mouse, the naïve signature was from (Kaech et al., 2002), the CD8 T cell activation signature was from (Sarkar et al., 2007), the cell cycle signature was from (Kowalczyk et al., 2015), the TRM signature was from (Beura et al., 2018), the bystander signature was from (Mognol et al., 2017), and the effector-like and terminally exhausted signatures were from (Miller et al., 2019). For human, the naïve and activation signatures were derived from (Akondy et al., 2017), and the exhaustion signature was obtained from (Sade-Feldman et al., 2018).

To create the plot shown in FIG. 9C, all cells were merged into a single data object and normalized to 10,000 counts per cell. Only cells with at least one alpha and one beta chain were included. Then each count was logarithmized according to log(1+X), where X is the gene count, and each gene was standardized to unit variance and zero mean. Given a signature, a score was calculated for each cell with Scanpy's ‘score_genes’ function. The average of the cell scores was calculated for each sample.

Machine Learning

Classification of tumor-matching cells in mouse (FIGS. 2A-2B) was conducted using L2 regularized logistic regression using the Scikit-learn package in python version 2.8 (Pedregosa F., 2011). Plots were generated using matplotlib. For the Logistic regression, the liblinear solver was used with a 12 penalty and C parameter set to 0.02.

Calculation of AUC

For each gene in each patient, the area under the curve in distinguishing TM cells from non-TM cells was computed with the AUC( ) function of the R DescTools package. To construct an input for the AUC( ) function, we calculated a vector of (1-Specificity) values and a vector of corresponding Sensitivity values from 39 potential expression level thresholds for dividing the two populations. For each gene in each patient, the 39 thresholds were every 5^{th} percentile expression of the gene (21 values including 0^{th} percentile and 100^{th} percentile) combined with 18 evenly spaced expression values between the minimum and maximum, to account for heavily skewed distributions in which useful thresholds may lie above the 95^{th} or below the 5^{th} percentile. To these input vectors we added (0,0) and (1,1), representing the trivial options of selecting none and all of the cells as TM, respectively.

Similarity of TM Component Across Patients

Similarity between samples in terms of the power of a transcript in distinguishing the TM component from the non-TM component was computed in FIG. 5C via pairwise correlation of gene transcript AUC values for selecting the TM component. The AUC for each gene transcript in each patient was calculated as described above, and all pairwise combinations between patient samples were plotted for each gene, resulting in (₂⁶)=15 points per gene. Negation markers are represented by AUC values<0.5 when selecting the TM component. To mitigate the x axis being arbitrarily biased toward the patients appearing first in the data, x and y coordinates were switched for each point with a probability of 0.5 and a random seed set to 27 in R. With the function stat_cor( ) from R package ggpubr, Pearson correlation statistics were computed for the resultant x and y values, stratified by whether each sample pair was within the same patient or across different patients. The plot is restricted to transcripts that were measured in all six patient samples.

COMET

COMET (Delaney et al., 2019) runs were conducted with version 0.1.12 and the default X parameter (0.15) and with the L parameter set to the minimum of (1) 10*K and (2) 0.35*N, where K is the number of TM cells and N is the total number of cells with at least one alpha and one beta chain annotated, to account for our willingness to allow for greater levels of contamination in the identified tumor-matching samples than allowed by default.

The full list of unranked markers from COMET are provided in Table S4 for mouse, and Table S10 for human. In these files, the COMET-determined threshold value (column labeled “cutoff_val”) is indicated for each marker when used as a positive marker or a negation marker. Negation markers are labeled as “marker negation”, whereas positive markers are simply listed at “marker”. For positive markers, a cell is predicted to be TM if its gene expression is above the threshold. For negation markers, a cell is predicted to be TM if its gene expression is lower than the threshold. In COMET's original output, negation markers are multiplied by (−1). We therefore took the absolute value of all reported thresholds in the output tables to increase clarity. Since “negation” does not necessarily equate to no expression for a marker, throughout the text cells deemed positive for a “negation marker” are referred to as “marker low” instead of “marker negative.”

Ranking Singleton Human Markers

Leading candidate markers for follow-up analysis from the human samples were determined by the number of patient samples in which a given marker reached significance in COMET (q<0.05). From the input list (Chihara et al., 2018), we removed CD8A because this is a lineage defining marker and therefore not ideal for separating the TM and non-TM components, along with cytokines CCL4, CCL5, and MIF in order to strictly consider surface-expressed markers. 15 markers derived from this filtered list had q<0.05 in the majority of patient samples (4 of 6) and were considered for follow-up analysis. These 15 candidate markers were ranked in order of descending AUC in distinguishing TM cells from non-TM cells for each patient, and ranks were averaged across patients to reach a summary continuous ranking for each of the 15 markers (Table S12). The top four on this list were negation markers for LTB, CCR7, GYPC, and FLT3LG, meaning that low or negative expression of these markers using a COMET-determined threshold is associated with TM cells.

Empirical p-Values and Confidence Intervals for Gate Performance

Confidence intervals for gate AUC, sensitivity, and specificity were determined by 10,000 iterations of random bootstrap resampling with replacement in the pooled CD8⁺ blood cell population and separately with respect to each individual patient blood sample. The 95% confidence intervals given go from the 2.5^{th} percentile to the 97.5^{th} percentile of 10,000 bootstrapped recalculations of AUC, sensitivity, and specificity. A null distribution for each gate was generated iteratively through each resample by permuting the “tumor-matching” labels and calculating the AUC, sensitivity, and specificity from the resultant datasets. These distributions represented the null hypothesis that each given marker was sorting TM cells from non-TM cells by chance alone. Reported empirical p-values<0.0001 reflect the observation that the point estimate for the marker's AUC, sensitivity, or specificity was never observed in the 10,000 iterations of the null.

Ranking of Combinatorial Marker Gates

All possible one, two, three, and four gene logical gates were enumerated from the four top-ranking markers in the patient samples (CCR7^{low}, FLT3LG^{low}, GYPC^{low} and LTB^{low}) and evaluated for their sensitivity (e.g. capture rate) and specificity (e.g. contamination rate) in isolating the TM component in each patient at a universal threshold of 0.001 UMIs. The optimal threshold to discriminate these two populations must be calibrated to the distribution of read counts as well as the target sensitivity and specificity. We used COMET to determine the optimal threshold for each marker in each patient sample (Table S14), and chose a universal threshold of 0.001 following manual inspection (COMET-derived thresholds averaged across patient samples were 0.001, 0.001, 0.832, and 0.332 for CCR7^{low}, FLT3LG^{low}, GYPC^{low} and LTB^{low}, respectively, and any threshold between 0 and 1 is functionally equivalent when applied to count data). To identify the best-performing combinatorial gate, we computed a penalty for each gate based on both its distance from perfect sensitivity and specificity as well as its balance between the two metrics. To calculate this penalty, we first computed the Euclidean distance from perfect sensitivity and specificity (corresponding to (0,1) on ROC curve plots) to the point on the plot representing that gate's sensitivity and specificity in the pool of CD8⁺ cells across all patient blood samples. To this Euclidean distance, we added the difference between the gate's sensitivity and specificity in the pool of CD8⁺ T cells across all patient blood samples in order to promote the selection of the most balanced gate. This process identified [(CCR7^{low} and FLT3LG^{low} and GYPC^{low}) or LTB^{low}] as the best-performing and most balanced gate (lowest penalty).

Quantification and Statistical Analysis

Flow Cytometry Validations in Mouse

Statistical analyses for flow cytometry data were performed with Prism software (GraphPad), and p-values of less than 0.05 were considered statistically significant. Multiple t tests were performed using the Holm-Sidak method with alpha=0.05. Each row was analyzed individually, without assuming a consistent SD. Asterisks indicating significance in the Figures correspond to p<0.05 (*), p<0.01 (**), and p<0.001 (***). Statistical tests used for computational analyses are indicated in the corresponding Figure Legends and Methods sections. Exact p values for significant comparisons are indicated in the Figure Legends and Supplemental Tables.

Resource Availability

The gene expression scRNA seq data for patients K409 and K411 (initial blood/tumor pair) and the TCR data (for tumor) can be found on GEO with accession number GSE148190 (Mahuron et al., 2020). The scRNA seq generated during this study will be deposited on GEO.

Description of Supplemental Material

Tables S1-S15 cited herein are available online at https://rupress.org/jem/article/218/4/e20200920/211836/Single-cell-analyses-identify-circulating-anti, which are incorporated by reference herein in their entirety. FIG. 7 is associated with FIG. 1, and provides additional details characterizing the single-cell RNA seq discovery dataset in mice. FIG. 8 is associated with FIG. 2, and provides additional information about the COMET output, flow cytometry validations in mice, and combinations of NKG2D, CX3CR1, and CD39 in our flow cytometry data and CITE seq data. FIG. 9 is associated with FIG. 3, and provides additional comparisons between matching clones in blood and tumor in replicate mice. FIG. 10 is associated with FIGS. 4 and 6, and provides supporting information regarding the single-cell RNA seq in the melanoma patients. FIG. 11 is associated with FIGS. 4-6, and details results from alternative methods to identify matching clones based on the TCR in melanoma patients. Table 51 shows up-regulated genes for each Seurat cluster in mouse integrated blood and MC38 tumor samples. Table S2 shows up-regulated genes for TM and non-TM CD8+ T cells in the peripheral blood of mice with MC38 tumors. Table S3 is a full list of pathways and signatures enriched in TM and in non-TM CD8+ T cells from the peripheral blood of mice with MC38 tumors. Table S4 shows significance measures calculated with COMET in sorting TM from non-TM CD8+ T cells in the blood of mice with MC38 tumors. Table S5 shows the sensitivity and specificity of all possible gates made from combinations of NKG2D, CD39, and CX3CR1, measured by CITE seq in mice. Table S6 lists DE genes between TM cells in blood and blood-matching cells in tumor. Table S7 shows clinical parameters for patient samples. Table S8 shows up-regulated genes for each Seurat cluster in human integrated blood and initial tumor samples. Table S9 shows transcript AUC performance, delineated by melanoma patient sample. Table S10 shows significance measures calculated with COMET for all transcripts in sorting TM from non-TM CD8+ T cells in the blood of melanoma patients. Table S1l shows similarities and differences in COMET-identified markers to identify TM cells in mice with MC38 tumors compared with human melanoma patients. Table S12 lists the 16 transcripts that were significant in at least four patient samples, ordered by average ranking of AUC. Table S13 lists empirical significance values and 95% confidence intervals for the AUC, sensitivity, and specificity of featured gates in each patient sample. Table S14 lists sensitivity and specificity values for all possible transcriptional marker combinations of CCR7low, GYPClow, FLT3LGlow, and LTBlow, delineated by patient sample. Table S15 lists empirical significance values and 95% confidence intervals for the AUC, sensitivity, and specificity of featured gates in each patient sample using the intersection of GLIPH2 and iSMART.

References cited herein are fully identified in Pauken et al, “Single-cell analyses identify circulating anti-tumor CD8 T cells and markers for their enrichment,” bioRiv preprint doi: https://doi.org/10.1101/2020.09.30.294959, posted Oct. 1, 2020.

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments described herein may be employed.

What is claimed is:

1. A method of treating cancer in a patient in need thereof, the method comprising:
isolating tumor-matching T cells ex vivo from blood obtained from the patient, thereby producing isolated tumor-matching T cells;
(ii) expanding the isolated tumor-matching T cells ex vivo, thereby producing expanded tumor-matching T cells; and
(iii) administering the expanded tumor-matching T cells to the patient, thereby treating the cancer.

2. The method of claim 1, wherein the tumor-matching T cell are CD8⁺ T cells.

3. The method of claim 1, wherein the tumor-matching T cells have an increased expression of at least one biomarker selected from the group consisting of KLRD1, NKG2D, KLRK1, CXCR3, CD39, LGAS1, and LGALS3.

4. The method of claim 1, wherein the tumor-matching T cells have a decreased expression of at least one biomarker selected from the group consisting of LTB, CCR7, GYPC, and FLT3LG.

5. The method of claim 1, wherein the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG.

6. The method of claim 1, wherein the tumor-matching T cells have an increased expression of KLRD1, NKG2D, and KLRK1; an increased expression of CXCR3, CD39, LGAS1, and LGALS3; and a decreased expression of LTB, CCR7, GYPC, and FLT3LG.

7. The method of claim 1, further comprising administering to the patient an effective amount of an immunotherapeutic agent, an anti-cancer agent, or a combination of thereof.

8. The method of claim 7, wherein the immunotherapeutic agent is a checkpoint inhibitor or an immunomodulator.

9. The method of claim 7, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof

10. The method of claim 1, wherein the cancer is melanoma.

11. A pharmaceutical composition comprising expanded tumor-matching T cells and a pharmaceutically acceptable excipient; wherein the tumor-matching T cells are made by a process comprising the steps of:
(i) isolating tumor-matching T cells ex vivo from blood obtained from a patient, thereby producing isolated tumor-matching T cells; and
(ii) expanding the isolated tumor-matching T cells ex vivo, thereby producing expanded tumor-matching T cells.

12. The pharmaceutical composition of claim 11, wherein the tumor-matching T cell are CD8⁺ T cells.

13. The pharmaceutical composition of claim 11, wherein the tumor-matching T cells have an increased expression of at least one biomarker selected from the group consisting of KLRD1, NKG2D, KLRK1, CXCR3, CD39, LGAS1, and LGALS3.

14. The pharmaceutical composition of claim 11, wherein the tumor-matching T cells have a decreased expression of at least one biomarker selected from the group consisting of LTB, CCR7, GYPC, and FLT3LG.

15. The pharmaceutical composition of claim 11, wherein the tumor-matching T cells have an increased expression of at least one biomarker selected from the group consisting of KLRD1, NKG2D, and KLRK1; an increased expression of at least one biomarker selected from the group consisting of CXCR3, CD39, LGAS1, and LGALS3; and a decreased expression of at least one biomarker selected from the group consisting of LTB, CCR7, GYPC, and FLT3LG.

16. A method of treating cancer in a patient in need thereof, the method comprising administering to the patient an effective amount of an immunotherapeutic agent, an anticancer agent, or a combination thereof; wherein the blood of the patient comprises a tumor-matching T cell having an increased expression level of at least one biomarker selected from the group consisting of KLRD1, NKG2D, KLRK1, CXCR3, CD39, LGAS1, and LGALS3; and/or a decreased expression level of at least one biomarker selected from the group consisting of LTB, CCR7, GYPC, and FLT3LG.

17. The method of claim 16, wherein the tumor-matching T cell has an increased expression level of one or more of KLRD1, NKG2D, and KLRK1; an increased expression level of one or more of CXCR3, CD39, LGAS1, and LGALS3; and a decreased expression level of one or more of LTB, CCR7, GYPC, and FLT3LG.

18. The method of claim 16, wherein the tumor-matching T cell has an increased expression level of KLRD1, NKG2D, and KLRK1; an increased expression level of CXCR3, CD39, LGAS1, and LGALS3; and a decreased expression level of LTB, CCR7, GYPC, and FLT3LG.

19. The method of claim 16, wherein the immunotherapeutic agent is a checkpoint inhibitor or an immunomodulator.

20. The method of claim 16, wherein the immunotherapeutic agent is pembrolizumab, nivolumab, cemiplimab, atezolizumab, avelumab, durvalumab, ipilimumab, aldesleukin, granulocyte-macrophage colony-stimulating factor, interferon alfa-2a, interferon alfa-2b, peginterferon alfa-2b, polyinosinic-polycytidylic acid-poly-1-lysine carboxymethylcellulose, pexidartinib, and a combination of two or more thereof
